(19) United States
(12) Patent Application Publication
(10) Pub. No.: US 2018/0273528 A1
(43) Pub. Date: Sep. 27, 2018

(54) IMIDAZO[4,5-b]PYRIDINE DERIVATIVES, A PROCESS FOR THEIR PREPARATION AND PHARMACEUTICAL COMPOSITIONS CONTAINING THEM

(71) Applicants: LES LABORATOIRES SERVIER, SURESNES CEDEX (FR); VERNALIS (R&D) LTD, Winnersh (GB)

(72) Inventors: Balázs BÁLINT, FÓT (HU); András KOTSCHY, Törökbálint (HU); Melinda SIPOS, Budapest (HU); Csaba WÉBER, Pilisszentlászló (HU); Nicolas FOLOPPE, Cambridge (GB); David WALMSLEY, Ely (GB); Michael BURBRIDGE, Verneuil Sur Seine (FR); Francisco Humberto CRUZALEGUI, Brax (FR)

(21) Appl. No.: 15/763,248

(22) PCT Filed: Sep. 30, 2016
(86) PCT No.: PCT/EP2016/073395

§ 371 Date: Mar. 26, 2018

(30) Foreign Application Priority Data
Sep. 30, 2015 (FR) 15/59252

(51) Int. Cl. C07D 471/04; A61K 31/444; A61K 45/06; A61P 35/00

(52) U.S. Cl. CPC C07D 471/04; A61P 35/00; A61K 45/06; A61K 31/444

(57) ABSTRACT

Compounds of formula (I):

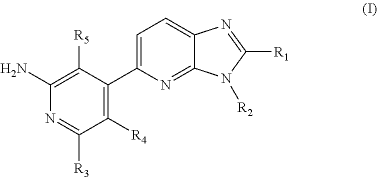

wherein R₁, R₂, R₃, R₄, and R₅ are as defined in the description.

Medicinal products containing the same which are useful in treating cancer, neurodegenerative disorders and metabolic disorders.

The present invention relates to new imidazo[4,5-b]pyridine derivatives, to a process for their preparation and to pharmaceutical compositions containing them.

The compounds of the present invention are new and have very valuable pharmacological characteristics in the field of oncology.

The present invention relates to the use of dual DYRK1/CLK1 inhibitors in the treatment of cancer, neurodegenerative disorders and metabolic disorders.

In cancer, the dual-specificity tyrosine-phosphorylation-regulated kinases DYRK1A and DYRK1B have been demonstrated to control several pathways that enhance cancer cell proliferation, migration and metastasis, induce resistance to cell death and repress responses to conventional and targeted anti-cancer therapies [Abbassi et al, Pharmacol Ther. 2015; 151:87-98; Ionescu et al, Mini Rev Med Chem. 2012; 12(13):1315-29; Friedman et al, J Cell Biochem. 2007; 102(2):274-9; Yoshida et al, Biochem Pharmacol. 2008; 76(11):1389-94]. Reported substrates of DYRK1A that are involved in this regulation of cancer progression and resistance to therapy include the transcription factors GLI1, STAT3 and FOXO1 [Mao et al, J Biol Chem. 2002; 277(38):35156-61; Matsuo et al, J Immunol Methods 2001; 247:141-51; Woods et al, Biochem J. 2001; 355(Pt 3): 597-607]. DYRK1A is also believed to stabilise cancer-associated tyrosine kinase receptors such as EGFR and FGFR via interaction with the protein Sprouty2 [Ferron et al, Cell Stem Cell. 2010; 7(3):367-79; Aranda et al, Mol Cell Biol. 2008; 28(19):5899-911]. DYRK1A, and also DYRK1B, have been shown to be required for the induction of cell quiescence in response to treatment of cancer cells by chemotherapeutic agents and targeted therapies. This is important since it is known that quiescent cancer cells are relatively insensitive to most anti-cancer drugs and radiation [Ewton et al, Mol Cancer Ther. 2011; 10(11):2104-14; Jin et al, J Biol Chem. 2009; 284(34):22916-25]. For example, DYRK1A activates the DREAM multisubunit protein complex, which maintains cells in quiescence and protects against apoptosis [Litovchick et al, Genes Dev. 2011; 25(8):801-13]. DYRK1B has been demonstrated to prevent cell-cycle exit in response to chemotherapy via phosphorylation of Cyclin D1 [Zou et al, J Biol Chem. 2004; 279(26):27790-8]. DYRK1B has also been shown to protect against chemotherapy through a reduction in reactive oxygen species content [Hu et al, Genes Cancer. 2010; 1(8):803-811].

It is thus clear that the use of DYRK1A/DYRK1B inhibitors would constitute a novel anti-cancer treatment in a wide variety of cancers when used either alone or in combination with conventional therapy, radiation or targeted therapies as a strategy to combat resistance.

The role of DYRK1A in neurological disorders is well established. DYRK1A is associated with neurodegenerative disorders such as Alzheimer's, Parkinson's and Huntington's diseases, as well as with Down's syndrome, mental retardation and motor defects and [Abbassi et al, Pharmacol Ther. 2015; 151:87-98; Beker et al, CNS Neurol Disord Drug Targets. 2014; 13(1):26-33; Dierssen, Nat Rev Neurosci. 2012 December; 13(12):844-58]. DYRK1A has been identified as a major kinase phosphorylating the microtubule-associated protein TAU, leading to the formation of neurotoxic neurofibrillary tangles and neurodegeneration as seen in Alzheimer's [Azorsa et al, BMC Genomics. 2010; 11:25]. DYRK1A also alters the splicing of TAU pre-mRNA leading to an imbalance between TAU isoforms which is sufficient to cause neurodegeneration and dementia [Liu et al, Mol Neurodegener. 2008; 3:8]. It is not surprising, therefore, that DYRK1A is believed to be causally involved in the development of Alzheimer-like neurodegenerative diseases in Down Syndrome patients, where three copies of the DYRK1A gene are present on chromosome 21. In these individuals, increased DYRK1A activity also causes premature neuronal differentiation and a decrease in mature neurones [Hämerle et al, Development. 2011; 138(12):2543-54].

It is thus clear that the use of DYRK1A inhibitors would offer a novel therapeutic approach for the treatment of neurodegenerative disorders, in particular Alzheimer's disease, as well as for other neurological conditions such as Down's syndrome.

The CDC2-like kinase (CLK) family contains four isoforms (CLK1-4) which are important in regulating the function of the spliceosome complex [Fedorov et al, Chem Biol. 2011; 18(1):67-76]. This complex, comprised of small nuclear RNAs (snRNA) and a large number of associated proteins, regulates the splicing of pre-mRNAs to give mature protein-encoding mRNAs. CLK1 is known to regulate the activity of the spliceosome via phosphorylation of the constituent serine-arginine-rich (SR) proteins [Bullock et at, Structure. 2009; 17(3):352-62]. By controlling the activity of the spliceosome in this way, many genes are able express more than one mRNA leading to diversity in the translated proteins. The alternative protein isoforms transcribed from the same gene will often have different activities and physiological functions. Deregulation of alternative splicing has been linked to cancer, where a number of cancer-related proteins are known to be alternatively spliced [Druillennec et al, J Nucleic Acids. 2012; 2012:639062]. An example of an alternatively spliced protein in cancer is Cyclin D1, important for the progression of cancer cells through the cell cycle [Wang et al, Cancer Res. 2008; 68(14):5628-38].

It is thus clear that the use of CLK1 inhibitors would constitute a novel anti-cancer treatment in a wide variety of cancers when used either alone or in combination with conventional therapy, radiation or targeted therapies.

Alternative splicing regulated by CLK1 has also been described to play a role in neurodegenerative diseases, including Alzheimer's and Parkinson's, via phosphorylation of the SR proteins of the spliceosome [Jain et al, Curr Drug Targets. 2014; 15(5):539-50]. In the case of Alzheimer's, CLK1 is known to regulate the alternative splicing of the microtubule-associated protein TAU leading to an imbalance between TAU isoforms which is sufficient to cause neurodegeneration and dementia [Liu et al, Mol Neurodegener. 2008; 3:8].

It is thus clear that the use of CLK1 inhibitors would offer a novel therapeutic approach for the treatment of neurodegenerative disorders, in particular Alzheimer's disease, as well as for other neurological conditions such as Parkinson's.

In the treatment of both cancer and neurological disease, there is thus undoubtedly an urgent need for compounds which potently inhibit the DYRK1 and CLK1 kinases whilst not affecting other closely-related kinases. The DYRK1 and CLK1 kinases are members of the CMGC group, which includes the CDK and the GSK kinases, the chronic inhibition of which is believed to be a cause of toxicity to the patient. For example, common toxicities observed in the clinic with CDK inhibition are similar to those observed with conventional cytotoxic therapy, and include hematologic toxicity (leukopenia and thrombocytopenia), gastrointestinal toxicity (nausea and diarrhea), and fatigue [Kumar et al, Blood. 2015; 125(3):443-8]. The present invention describes a new class of DYRK1/CLK1 inhibitors which are highly selective for DYRK1 and CLK1 over these other kinases and which would thus be suitable for use in the treatment of these pathologies.

Diabetes type 1 and type 2 both involve deficiency of functional pancreatic insulin-producing beta cells. Restoring functional beta-cell mass is thus an important therapeutic goal for these diseases which affect 380 million people worldwide. Recent studies have shown that DYRK1A inhibition promotes human beta-cell proliferation in vitro and in vivo and, following prolonged treatment, can increase glucose-dependent insulin secretion [Dirice et a, Diabetes. 2016; 65(6):1660-71; Wang et al, Nat Med. 2015; 21(4):383-8]. These observations clearly suggest that the use of potent and selective DYRK1A inhibitors would offer a novel therapeutic approach for the treatment and/or prevention of metabolic disorders including diabetes and obesity.

The present invention relates more especially to compounds of formula (I):

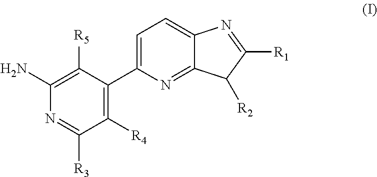

wherein.
R₁ represents a cyano group, a halogen atom, or a linear or branched (C₁-C₆)alkyl group optionally substituted by from one to three halogen atoms,
R₂ represents a hydrogen, a linear or branched (C₁-C₆)alkyl group, a linear or branched (C₂-C₆)alkenyl group, a linear or branched (C₂-C₆)alkynyl group, Cy₁, —(C₁-C₆)alkylene-[O]ₙ—Cy₁ group, —(C₁-C₆)alkenylene-[O]ₙ-Cy₁ group. —(C₁-C₆)alkylene-NR-Cy₁ group, —(C₁-C₆)alkylene-S-Cy₁ group, —(C₀-C₆)alkylene-Cy₂-Cy₁ group, or —Cy₂-(C₁-C₆)alkylene-Cy₁ group, it being understood that the alkyl and alkylene moieties defined hereinbefore may be linear or branched,
R represents a hydrogen or a linear or branched (C₁-C₆)alkyl group,
n is an integer equals to 0 or 1,
R₃ represents a hydrogen atom, a halogen atom, —NR₆R₆′, —NH—(C₀-C₆)alkylene-Cy₃, —NH—CO—(C₀-C₆)alkylene-Cy₃, —NH—CO—(C₀-C₆)alkylene-O-Cy₃,
R₄ and R₅, each independently of the others, represent a hydrogen or a halogen atom,
R₆ and R₆′, each independently of the others, represent a hydrogen or a linear or branched (C₁-C₆)alkyl group,
Cy₁, Cy₂ and Cy₃, independently of one another, represent a cycloalkyl group, a heterocycloalkyl group, an aryl or an heteroaryl group,
   it being understood that:
“aryl” means a phenyl, naphthyl, biphenyl or indenyl group,
“heteroaryl” means any mono- or bi-cyclic group composed of from 5 to 10 ring members, having at least one aromatic moiety and containing from 1 to 4 hetero atoms selected from oxygen, sulphur and nitrogen,
“cycloalkyl” means any mono- or bi-cyclic, non-aromatic, carbocyclic group containing from 3 to 11 ring members, which may include fused, bridged or spiro ring systems,
“heterocycloalkyl” means any mono- or bi-cyclic, non-aromatic, condensed or spiro group composed of from 3 to 10 ring members and containing from 1 to 3 hetero atoms selected from oxygen, sulphur, SO, SO₂ and nitrogen, which may include fused, bridged or spiro ring systems,
“—(C₀-C₆)alkylene-” refers either to a covalent bond (—C₀alkylene-) or to an alkylene group containing 1, 2, 3, 4, 5 or 6 carbon atoms,
   it being possible for the aryl, heteroaryl, cycloalkyl and heterocycloalkyl groups so defined and the alkyl, alkenyl, alkynyl, alkylene, alkenylene to be substituted by from 1 to 4 groups selected from linear or branched (C₁-C₆)alkyl, linear or branched (C₂-C₆)alkenyl group, linear or branched (C₂-C₆)alkynyl group, linear or branched (C₁-C₆)alkoxy, linear or branched (C₁-C₆)alkyl-S—, hydroxy, oxo (or N-oxide where appropriate), nitro, cyano, —C(O)—OR′, —C(O)—R′, —O—C(O)—R′, —C(O)—NR′R″, —NR′—C(O)—R″, —NR′R″, linear or branched (C₁-C₆)polyhaloalkyl, difluoromethoxy, trifluoromethoxy, or halogen, it being understood that R′ and R″ independently of one another represent a hydrogen atom or a substituted linear or branched (C₁-C₆)alkyl group,
   to their enantiomers and diastereoisomers, and to addition salts thereof with a pharmaceutically acceptable acid or base.

Among the pharmaceutically acceptable acids there may be mentioned, without implying any limitation, hydrochloric acid, hydrobromic acid, sulphuric acid, phosphonic acid, acetic acid, trifluoroacetic acid, lactic acid, pyruvic acid, malonic acid, succinic acid, glutaric acid, fumaric acid, tartaric acid, maleic acid, citric acid, ascorbic acid, oxalic acid, methanesulphonic acid, camphoric acid etc.

Among the pharmaceutically acceptable bases there may be mentioned, without implying any limitation, sodium hydroxide, potassium hydroxide, triethylamine, tert-butylamine etc.

Advantageously, R₁ represents a methyl or a cyano group.

In another embodiment of the invention, R₄ and R₅ each represent a hydrogen atom

Preferably, R₃ represents a NH₂ group.

Alternatively, R₃ represents a hydrogen atom.

In one embodiment, R₂ represents a hydrogen, a linear or branched (C₁-C₆)alkyl group, a linear or branched (C₂-C₆)alkenyl group, a linear or branched (C₂-C₆)alkynyl group, —(C₁-C₆)alkylene-O-Cy₁ group, —(C₁-C₆)alkenylene-[O]ₙ-Cy₁ group, —(C₁-C₆)alkylene-NR-Cy₁ group, —(C₁-C₆)alkylene-S-Cy₁ group, —(C₀-C₆)alkylene-Cy₂-Cy₁ group, or —Cy₂-(C₂-C₆)alkylene-Cy₁ group, it being understood that the alkyl and alkylene moieties defined hereinbefore may be linear or branched.

In another embodiment of the invention, R₂ represents Cy₁, a —(C₁-C₆)alkylene-Cy₁ group, —(C₀-C₆)alkylene-Cy₂-Cy₁ group, or —Cy₂-(C₁-C₆)alkylene-Cy₁ group. More preferably, R₂ represents:
a cycloalkyl group,
or a —(C₁-C₆)alkylene-cycloalkyl or a —(C₁-C₆)alkylene-phenyl group,
or a -cycloalkylene-phenyl group or a -cycloalkylene-(C₁-C₆)alkylene-phenyl group,
   wherein the cycloalkyl, cycloalkylene and phenyl groups so defined can be optionally substituted according to the definitions mentioned previously. Halogens, methoxy and methyl groups are the preferred substituents for the preceding groups.

In a third embodiment, R₂ represents a linear or branched (C₁-C₆)alkyl group, wherein the alkyl group so defined can be optionally substituted according to the definitions mentioned previously. Halogens and CH₃—S— are the preferred substituents for the alkyl group.

In a fourth embodiment, R₂ represents —(C₁-C₆)alkylene-O-Cy₁ group. More preferably, R₂ represents a —(C₁-C₆)alkylene-O-pyridinyl group, wherein the pyridinyl group so defined can be optionally substituted according to the definitions mentioned previously. Halogens and linear or branched (C₁-C₆)polyhaloalkyl groups are the preferred substituents for the pyridinyl group.

Preferred compounds according to the invention are included in the following group:
4-[2-methyl-3-(3-phenylcyclobutyl)-3H-imidazo[4,5-b]pyridin-5-yl]pyridine-2,6-diamine,
4-[3-(3,3-difluorocyclobutyl)-2-methyl-3H-imidazo[4,5-b]pyridin-5-yl]pyridine-2,6-diamine,
4-(3-{2-[(6-fluoropyridin-2-yl)oxy]ethyl}-2-methyl-3H-imidazo[4,5-b]pyridin-5-yl)pyridine-2,6-diamine,
4-{3-[(1R,2R)-2-benzylcyclopropyl]-2-methyl-3H-imidazo[4,5-b]pyridin-5-yl}pyridine-2,6-diamine,
4-[3-(3-fluorocyclobutyl)-2-methyl-3H-imidazo[4,5-b]pyridin-5-yl]pyridine-2,6-diamine,
4-(3-hexyl-2-methyl-3H-imidazo[4,5-b]pyridin-5-yl)pyridine-2,6-diamine,
4-(3-cyclobutyl-2-methyl-3H-imidazo[4,5-b]pyridin-5-yl)pyridine-2,6-diamine,
4-[3-(2-{[6-(difluoromethyl)pyridin-2-yl]oxy}ethyl)-2-methyl-3H-imidazo[4,5-b]pyridin-5-yl]pyridine-2,6-diamine,
4-[3-(5-methoxy-2,3-dihydro-1H-inden-2-yl)-2-methyl-3H-imidazo[4,5-b]pyridin-5-yl]pyridine-2,6-diamine,
4-(3-ethyl-2-methyl-3H-imidazo[4,5-b]pyridin-5-yl)pyridine-2,6-diamine,
4-[2-methyl-3-(2-{[6-(trifluoromethyl)pyridin-2-yl]oxy}ethyl)-3H-imidazo[4,5-b]pyridin-5-yl]pyridine-2,6-diamine,
4-{3-[2-(2-methoxycyclohexyl)ethyl]-2-methyl-3H-imidazo[4,5-b]pyridin-5-yl}pyridine-2,6-diamine,
4-(2-methyl-3-pentyl-3H-imidazo[4,5-b]pyridin-5-yl)pyridine-2,6-diamine,
4-(3-cyclohexyl-2-methyl-3H-imidazo[4,5-b]pyridin-5-yl)pyridine-2,6-diamine,
4-{2-methyl-3-[3-(methylsulfanyl)propyl]-3H-imidazo[4,5-b]pyridin-5-yl}pyridine-2,6-diamine,
4-{3-[(1R,2S)-2-benzylcyclopropyl]-2-methyl-3H-imidazo[4,5-b]pyridin-5-yl}pyridine-2,6-diamine,
4-{2-methyl-3-[2-(2-methylphenyl)ethyl]-3H-imidazo[4,5-b]pyridin-5-yl}pyridine-2,6-diamine,
4-(3-{2-[(6-chloropyridin-2-yl)oxy]ethyl}-2-methyl-3H-imidazo[4,5-b]pyridin-5-yl)pyridine-2,6-diamine,
4-(3-{(2R)-2-[(6-fluoropyridin-2-yl)oxy]propyl}-2-methyl-3H-imidazo[4,5-b]pyridin-5-yl)pyridine-2,6-diamine,
4-[2-methyl-3-(2,2,2-trifluoroethyl)-3H-imidazo[4,5-b]pyridin-5-yl]pyridine-2,6-diamine,
3-cyclopentyl-5-(2,6-diaminopyridin-4-yl)-3H imidazo[4,5-b]pyridine-2-carbonitrile,
4-(3-cyclopropyl-2-methyl-3H-imidazo[4,5-b]pyridin-5-yl)pyridine-2,6-diamine,
   their enantiomers and diastereoisomers, and addition salts thereof with a pharmaceutically acceptable acid or base.

The invention relates also to a process for the preparation of compounds of formula (I), which process is characterised in that there is used as starting material the compound of formula (II):

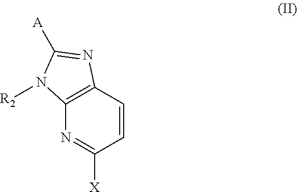

wherein A represents a halogen atom, or a linear or branched (C₁-C₆)alkyl group optionally substituted by from one to three halogen atoms. X represent a halogen atom, and R₂ is as defined in formula (I),
which compound of formula (II) is subjected to coupling with a compound of formula (III):

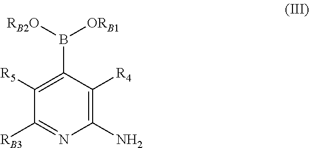

wherein:
R_{B1} and R_{B2} represent a hydrogen, a linear or branched (C₁-C₆) alkyl group, or R_{B1} and R_{B2} form with the oxygen atoms carrying them an optionally methylated ring.
R_{B3} represents a hydrogen or group NH₂,
R₄ and R₅ are as defined in formula (I),
   to yield compound of formula (IV):

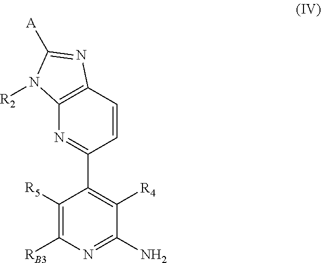

wherein A represents a halogen atom, or a linear or branched (C₁-C₆)alkyl group optionally substituted by from one to three halogen atoms, R_{B3} represents a hydrogen or group NH₂, and R₂, R₄ and R₅ are as defined in formula (I),
which compound of formula (IV):
may be reacted with Et₄NCN when A represents a halogen to yield the compounds of formula (I) wherein R₁=—CN, or
may be subjected to an aromatic nucleophilic substitution when R₂ represents a linear or branched HO—(C₁-C₆)alkylene group, and/or
may be subjected to an acylation in the presence of an acid derivative,
   to yield the compounds of formula (I),
   which compound of formula (I) may be purified according to a conventional separation technique, which is converted, if desired, into its addition salts with a pharmaceutically acceptable acid or base and which is optionally separated into its isomers according to a conventional separation technique,
   it being understood that, at any time considered appropriate in the course of the above-described process, certain groups (hydroxy, amino . . . ) of the reagents or intermediates of synthesis may be protected and then deprotected according to the requirements of synthesis.

The invention relates also to an alternative process for the preparation of compounds of formula (I), which process is characterised in that there is used as starting material the compound of formula (II′):

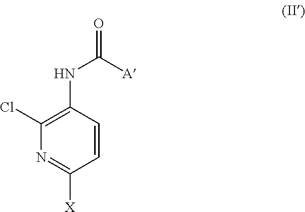

wherein A′ represents a linear or branched (C₁-C₆)alkyl group optionally substituted by from one to three halogen atoms, and X represents a halogen atom,
which compound of formula (II′) is subjected to coupling with a compound of formula (III):

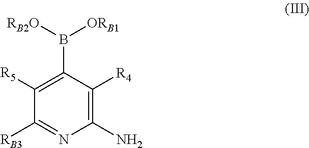

wherein:
R_{B1} and R_{B2} represent a hydrogen, a linear or branched (C₁-C₆) alkyl group, or R_{B1} and R_{B2} form with the oxygen atoms carrying them an optionally methylated ring,
R_{B3} represents a hydrogen or group NH₂,
R₄ and R₅ are as defined in formula (I),
   to yield compound of formula (IV′):

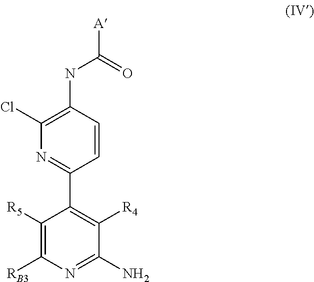

wherein.
A′ represents a linear or branched (C₁-C₆)alkyl group optionally substituted by from one to three halogen atoms,
R_{B3} represents a hydrogen or group NH₂,
R₄ and R₅ are as defined in formula (I),
   which compound of formula (IV′) is:
A) either subjected to a nucleophilic substitution in the presence of a compound of formula R₂—NH₂, wherein R₂ is as defined in formula (I) to yield the compound of formula (V′):

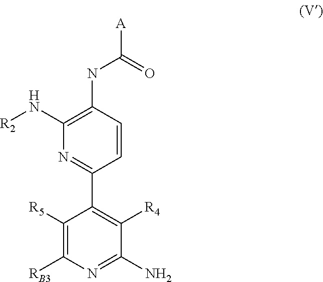

wherein:
   A′ represents a linear or branched (C₁-C₆)alkyl group optionally substituted by from one to three halogen atoms,
   R_{B3} represents a hydrogen or group NH₂,
   R₂, R₄ and R₅ are as defined in formula (I),
which compound of formula (V′) is submitted to an intramolecular reaction (ring closure) in acidic medium, to yield the compound of formula (I),
B) or converted into the corresponding imino sulfonate derivative of formula (VI′):

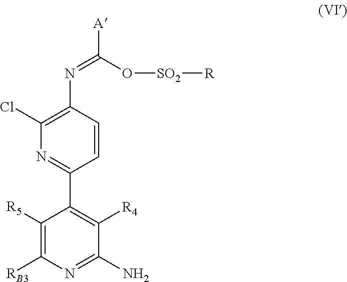

wherein:
   R is a linear or branched (C₁-C₆)alkyl group, an optionally substituted aryl, or a linear or branched polyhalogenated (C₁-C₆)alkyl group,
   A′ represents a linear or branched (C₁-C₆)alkyl group optionally substituted by from one to three halogen atoms,
   R_{B3} represents a hydrogen or group NH₂,
   R₄ and R₅ are as defined in formula (I),
which compound of formula (VI′) is further subjected to a nucleophilic substitution in the presence of a compound of formula R₂—NH₂, wherein R₂ is as defined in formula (I), to yield the compound of formula (VII′):

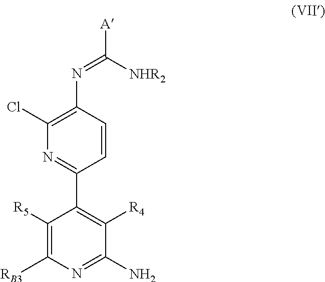

wherein:
   A′ represents a linear or branched (C₁-C₆)alkyl group optionally substituted by from one to three halogen atoms,
   R_{B3} represents a hydrogen or group NH₂,
   R₂, R₄ and R₅ are as defined in formula (I),
which compound of formula (VII′) is submitted to an intramolecular organometallic coupling reaction, to yield the compound of formula (I) wherein the definition of R, is limited to the one of A′,
   which compound of formula (I) may be purified according to a conventional separation technique, which is converted, if desired, into its addition salts with a pharmaceutically acceptable acid or base and which is optionally separated into its isomers according to a conventional separation technique,
   it being understood that, at any time considered appropriate in the course of the above-described process, certain groups (hydroxy, amino . . . ) of the reagents or intermediates of synthesis may be protected and then deprotected according to the requirements of synthesis.

The compounds of formulae (II), (II′), (III′) and the amine R₂—NH₂ are either commercially available or can be obtained by the person skilled in the art using conventional chemical reactions described in the literature.

Pharmacological study of the compounds of the invention has shown that they are powerful DYRK1/CLK1 inhibitors which are highly selective for DYRK1 and CLK1 over other kinases such as CDK9.

More especially, the compounds according to the invention will be useful in the treatment of chemo- or radio-resistant cancers.

Among the cancer treatments envisaged there may be mentioned, without implying any limitation, haematological cancer (lymphoma and leukemia) and solid tumors including carcinoma, sarcoma, or blastoma. There may be mentioned more preferably acute megakaryoblastic leukaemia (AMKL), acute lymphoblastic leukaemia (ALL), ovarian cancer, pancreatic cancer, gastrointestinal stromal tumours (GIST), osteosarcoma (OS), colorectal carcinoma (CRC), neuroblastoma and glioblastoma.

In another embodiment, the compounds of the invention will useful in the treatment of neurodegenerative disorders such as Alzheimer's, Parkinson's and Huntington's diseases, as well as with Down's syndrome, mental retardation and motor defects.

Alternatively, the compounds of the invention could be used in the treatment and/or prevention of metabolic disorders including diabetes and obesity.

The present invention relates also to pharmaceutical compositions comprising at least one compound of formula (I) in combination with one or more pharmaceutically acceptable excipients.

Among the pharmaceutical compositions according to the invention there may be mentioned more especially those that are suitable for oral, parenteral, nasal, per- or trans-cutaneous, rectal, perlingual, ocular or respiratory administration, especially tablets or dragées, sublingual tablets, sachets, paquets, capsules, glossettes, lozenges, suppositories, creams, ointments, dermal gels, and drinkable or injectable ampoules.

The dosage varies according to the sex, age and weight of the patient, the administration route, the nature of the therapeutic indication, or of any associated treatments, and ranges from 0.01 mg to 5 g per 24 hours in one or more administrations.

Furthermore, the present invention relates also to the combination of a compound of formula (I) with an anticancer agent selected from genotoxic agents, mitotic poisons, anti-metabolites, proteasome inhibitors, kinase inhibitors, signaling pathway inhibitors, phosphatase inhibitors, apoptosis inducers and antibodies, and also to pharmaceutical compositions comprising that type of combination and their use in the manufacture of medicaments for use in the treatment of cancer.

The combination of a compound of formula (I) with an anticancer agent may be administered simultaneously or sequentially. The administration route is preferably the oral route, and the corresponding pharmaceutical compositions may allow the instantaneous or delayed release of the active ingredients. The compounds of the combination may moreover be administered in the form of two separate pharmaceutical compositions, each containing one of the active ingredients, or in the form of a single pharmaceutical composition, in which the active ingredients are in admixture.

The compounds of the invention may also be used in combination with radiotherapy in the treatment of cancer.

### LIST OF ABBREVIATIONS

| Abbreviation | Name |
|---|---|
| Ac | acetyl |
| CDI | 1,1-carbonyldiimidazole |
| DCM | dichloromethane |
| DME | 1,2-dimethoxyethane |
| DMF | N,N-Dimethylformamide |
| DMSO | dimethyl sulfoxide |
| eq. | equivalent |
| Et | ethyl |
| HPLC-MS | liquid chromatography-mass spectrometry |
| Me | methyl |
| ⁿBu | n-butyl |
| ⁿBuPAd₂ | n-butyldiademantylphosphine |
| Ph | phenyl |
| PPh₃ | triphenylphosphine |
| ^{t}Bu | tert-butyl |
| TEA | triethylamine |
| TFA | trifuoroacetic acid |
| THF | tetrahydrofurane |

The following Preparations and Examples illustrate the invention without limiting it in any way.

### General Procedure 1

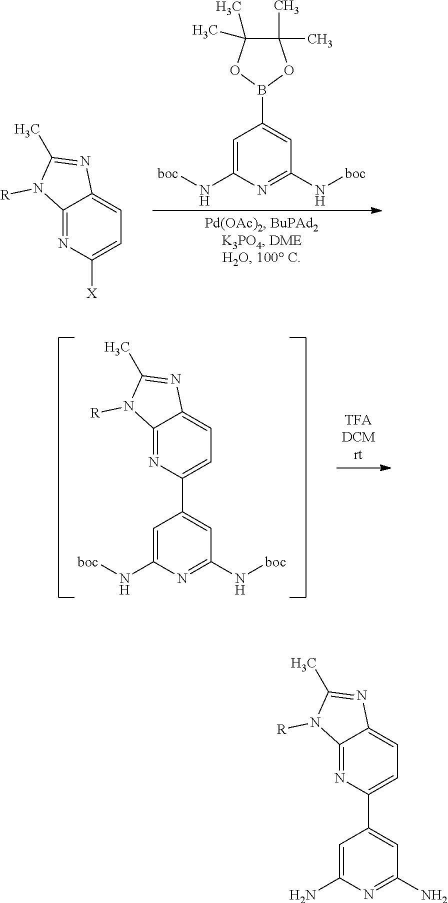

### Step A

1 eq. of the appropriate halide derivative, 1.2 eq. tert-butyl N-[6-(tert-butoxycarbonylamino)-4-(4,4,5,5-tetramethyl-1,3,2-dioxaborolan-2-yl)-2-pyridyl]carbamate (Preparation 1) and 3 eq. K₂CO₃ were dissolved in 1,2-dimethoxyethane-water 7:1 (8 mL/mmol). Then 0.05 eq. palladium acetate and 0.1 eq. ⁿBuPAd₂ were added and the mixture was heated at 100° C. under nitrogen in a microwave reactor until no further conversion was observed. Celite was added to the reaction mixture and the volatiles were evaporated under reduced pressure. The solid residue was purified via flash chromatography on silica gel using MeOH-containing 1% NH₃— and DCM as eluents.

### Step B

The product obtained in Step A was stirred in a mixture of DCM (5 mL/mmol) and TFA (5 mL/mmol) until no further conversion was observed. The volatiles were evaporated under reduced pressure, the solid residue was dissolved in ammonia solution (7N in methanol, 20 mL/mmol) and the volatiles were evaporated under reduced pressure again. The crude product was purified via preparative reversed phase chromatography using 5 mM aqueous NH₄HCO₃ solution and MeCN as eluents.

### General Procedure II

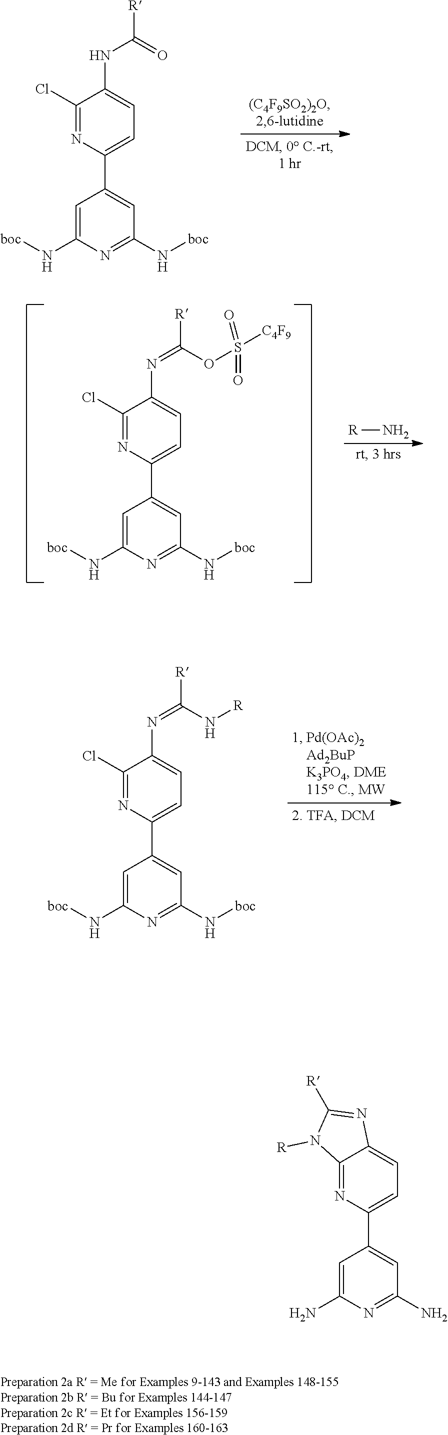

### Step A

1.0 eq. of the appropriate amide (Preparation 2a, Preparation 2b, Preparation 2c or Preparation 2d) and 5.0 eq. 2,6-lutidine were dissolved in dry DCM (0.10 M solution for Preparation 2). The DCM solution was cooled to 0° C. under nitrogen and DCM solution of 5.0 eq. nonafluorobutanesulfonic anhydride (1.5 M) was added dropwise. The reaction mixture was allowed to warm up to room temperature over 1 hour then 5 eq. of the appropriate amine was added in one portion and the mixture was stirred until no further conversion was observed. The DCM mixture was washed with water, dried over Na₂SO₄, concentrated under reduced pressure and purified via flash chromatography using dichloromethane and methanolic ammonia as eluents to give the amidine intermediate.

### Step B

1. eq. amidine intermediate from Step A was dissolved in 1,2-dimethoxyethane (0.15 M solution). 0.2 eq. Pd(OAc)₂, 0.4 eq. PBuAd₂, and 2 eq. K₃PO₄ were added and the reaction mixture was stirred under nitrogen at 115° C. in a microwave reactor until no further conversion was observed. The reaction mixture was concentrated under reduced pressure and purified via flash chromatography using dichloromethane and methanolic ammonia as eluents to yield the appropriate Boc-protected example.

### Step C

Starting from the product of Step B following General procedure I Step B the appropriate example was obtained.

### General Procedure III

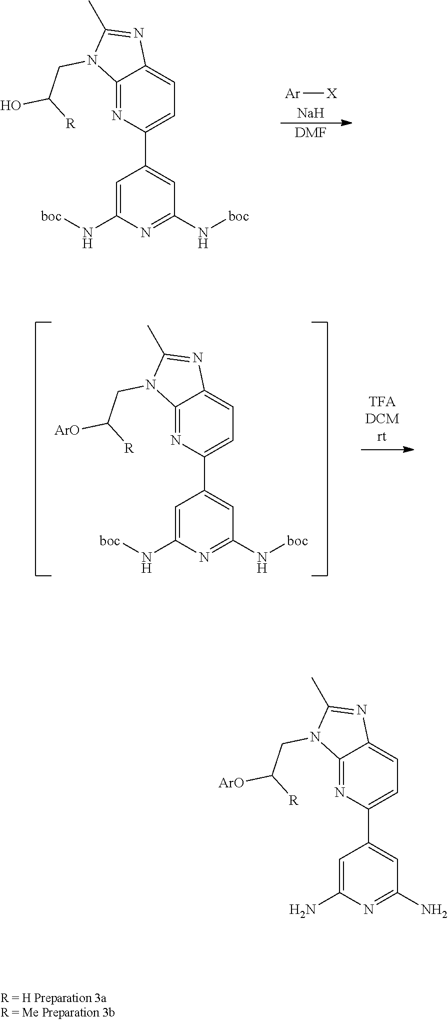

### Step A

To the solution of 1 eq. of Preparation 3a or Preparation 3b in dry DMF (0.25 M) under nitrogen 3 eq. sodium hydride was added and the resulting mixture was stirred at 0° C. for 15 min. Following the addition of 2 eq. of the appropriate aryl halide the mixture was stirred at 50° C. for 5 hours. If formation of the expected product was not observed by HPLC-MS at this point the reaction temperature was raised to 120° C. and stirring continued until no further conversion was observed. After cooling water was added to the reaction mixture and the aqueous phase was extracted with EtOAc. The combined organic phases were washed with brine, dried over anhydrous MgSO₄ and solvent was removed under reduced pressure. The crude product was purified by flash chromatography using DCM and MeOH as eluents to give the Boc-protected example.

### Step B

Starting from the product of Step A following General procedure I Step B the appropriate example was obtained.

### General Procedure IV

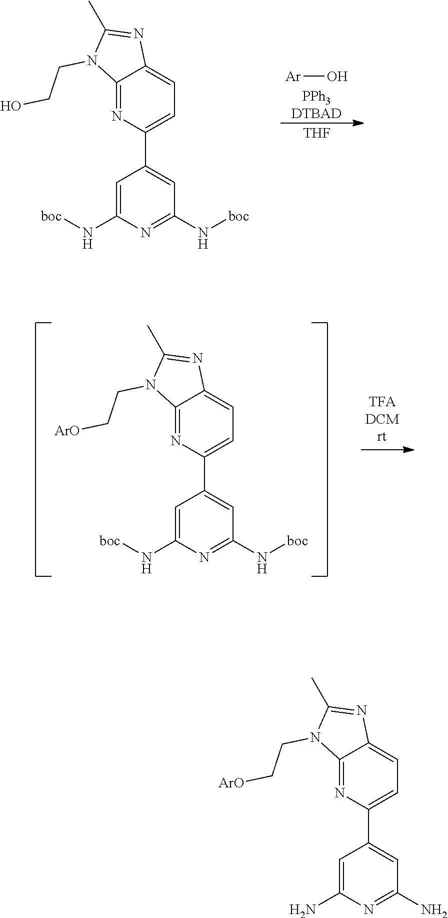

### Step A

1 eq. tert-butyl N-[6-(tert-butoxycarbonylamino)-4-[3-(2-hydroxyethyl)-2-methyl-imidazo[4,5-b]pyridin-5-yl]-2-pyridyl]carbamate (Preparation 3a), 2 eq. of the appropriate phenol derivative, 2 eq. PPh₃, and 2 eq. ditertbutyl azodicarboxylate was dissolved in THF (10 mL/mmol of Preparation 3a). and the mixture was stirred at 60° C. until no further conversion was observed. Celite was added to the reaction mixture and the volatiles were evaporated under reduced pressure. The solid residue was purified via flash chromatography on silica gel using MeOH-containing 1% NH₃— and DCM as eluents to give the appropriate Boc-protected example.

### Step B

Starting from the product of Step A following General procedure I Step B the appropriate example was obtained.

### General Procedure V

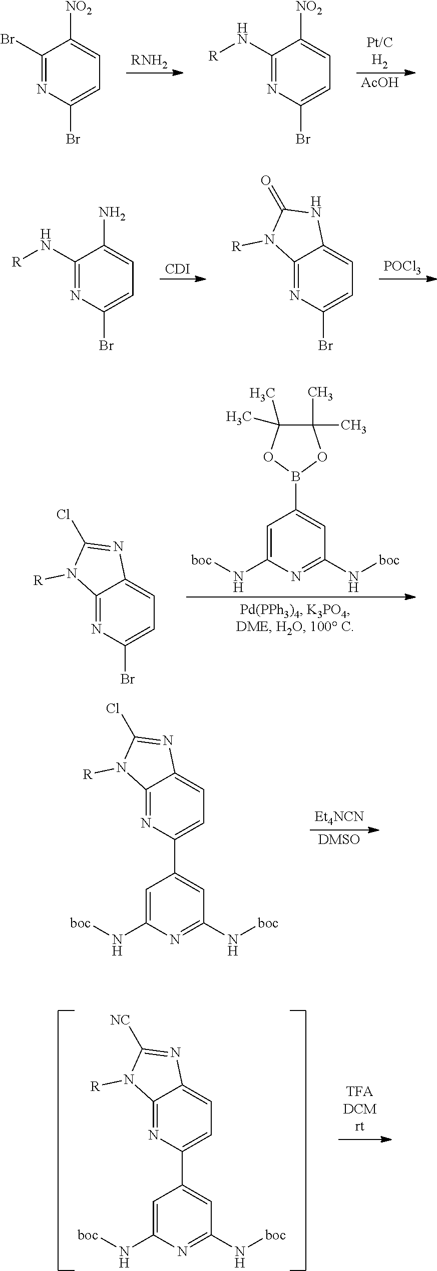

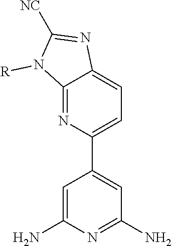

### Step A

The mixture of 1 eq. 2,6-dibromo-3-nitro-pyridine, 3.2 eq. K₂CO₃, and 1.05 eq. of the appropriate amine in 1,2-dichloroethane (0.17 M for the bromopyridine) was stirred at 50° C. until no further conversion was observed. Water was added to the mixture and the aqueous phase was separated and extracted three times with DCM. The combined organic layers were dried over MgSO₄, solvent was removed under reduced pressure and the crude product was purified by flash chromatography using dichloromethane and methanol as eluents to give the appropriate 2-amino-3-nitro-6-bromopyridine.

### Step B

1 eq. of the appropriate 2-amino-3-nitro-6-bromopyridine, 5 eq. Fe powder and 0.2 eq. NH₄Cl were stirred in a mixture of EtOH and water (3:1, 0.1M for 2-amino-3-nitro-6-bromopyridine) at 90° C. until no further conversion was observed. The reaction mixture was filtered through celite, and the solvent was removed under reduced pressure to give the appropriate 2,3-diamino-6-bromopyridine that was used without further purification.

### Step C

The mixture of 1 eq. of the appropriate N²-substituted 2,3-diamino-6-bromopyridine and 1.5 eq. CDI were stirred in dry THF (0.05M solution for 2,3-diamino-6-bromopyridine) until no further conversion was observed. The solvent was removed under reduced pressure and the crude product was purified by flash chromatography using dichloromethane and methanol as eluents to give the appropriate 5-bromo-2-oxo-1H-imidazo[4,5-b]pyridine.

### Step D

The mixture of 1 eq. 3-substituted-5-bromo-2-oxo-1H-imidazo[4,5-b]pyridine and POCl₃ (5 ml) was stirred at 108° C. until no further conversion was observed. POCl₃ was removed under reduced pressure. Dichloromethane and brine were added, organic phase was separated and the aqueous phase was extracted 2 times with dichloromethane. The combined organic layers were dried over MgSO₄, the solvent was removed under reduced pressure and the crude product was purified by flash chromatography using dichloromethane and methanol as eluents to give the appropriate 3-substituted-5-bromo-2-chloro-imidazo[4,5-b]pyridine.

### Step E

Starting from the appropriate 3-substituted-5-bromo-2-chloro-imidazo[4,5-b]pyridine and following the procedure described for Preparation 3a the appropriate 3-substituted-5-(2,6-bis(tert-butoxycarbamoyl)pyridin-4-yl)-2-chloro-imidazo[4,5-b]pyridine was obtained.

### Step F

The mixture of 1 eq. of the appropriate 3-substituted-5-(2,6-bis(tert-butoxycarbamoyl)pyridin-4-yl)-2-chloro-imidazo[4,5-b]pyridine and 1.05 eq. tetraethylammonium cyanide was stirred in DMSO (0.03M solution for imidazopyridine) until no further conversion was observed. The reaction mixture was poured onto water, the solid was filtered off, and the aqueous phase was extracted with chloroform. The organic layers were combined, dried over anhydrous MgSO₄ and the solvent was removed under reduced pressure. The crude product was purified by flash chromatography using dichloromethane and ethyl acetate as eluents to give the appropriate 3-substituted-5-(2,6-bis(tert-butoxycarbamoyl)pyridin-4-yl)-2-cyano-imidazo[4,5-b]pyridine.

### Step G

Starting from the product of Step F following General procedure I Step B the appropriate example was obtained.

### General Procedure VI

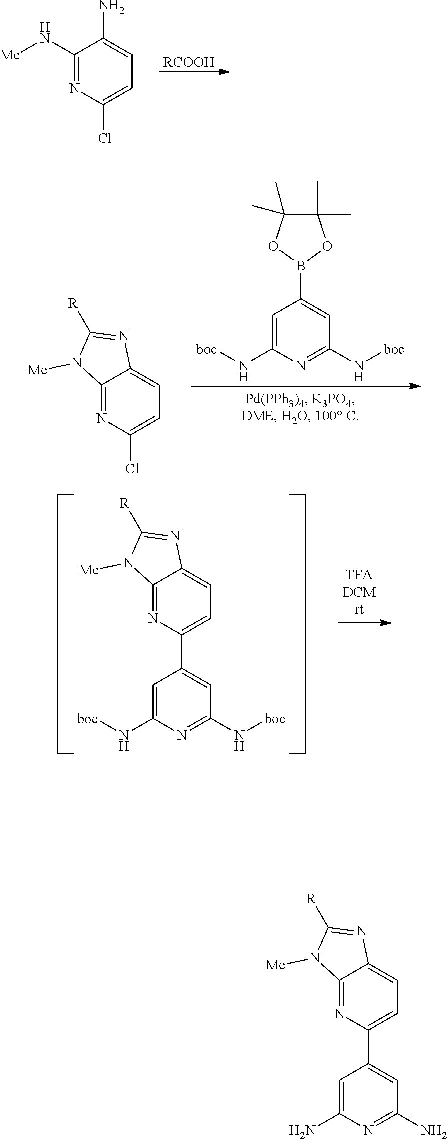

### Step A

1 eq. 6-chloro-2-methylamino-3-aminopyridine (Preparation 4) and 2.5 eq. of the appropriate acetic acid derivative were dissolved in toluene (1 mL/mmol) and the mixture was stirred at 85° C. until no further conversion was observed. The volatiles were evaporated under reduced pressure and the solid residue was purified via flash chromatography on silica gel using methanol and DCM as eluents to give 5-chloro-3-methyl-2-(trifluoromethyl)imidazo[4,5-b]pyridine

¹H NMR (500 MHz, DMSO-d₆) δ 8.39 (d, 1H), 7.57 (d, 1H), 3.95 (s, 3H) or 5-chloro-2-(difluoromethyl)-3-methyl-imidazo[4,5-b]pyridine—¹H NMR (500 MHz, DMSO-d₆) δ 8.27 (d, 1H), 7.46 (d, 1H), 7.44 (t, 1H), 3.9 (s, 3H).

### Step B

1 eq. of the product obtained in Step A, 1.3 eq. tert-butyl N-[6-(tert-butoxycarbonylamino)-4-(4,4,5,5-tetramethyl-1,3,2-dioxaborolan-2-yl)-2-pyridyl]carbamate (Preparation 1), and 2 eq. K₃PO₄ were dissolved in 1,2-dimethoxyethane (6 mL/mmol) then 0.1 eq Pd(PPh₃)₄ was added and the resulting mixture was heated at 100° C. under nitrogen using microwave irradiation until no further conversion was observed. Celite was added to the reaction mixture and the volatiles were evaporated under reduced pressure. The solid residue was purified via flash chromatography on silica gel using MeOH and DCM as eluents to give the Boc-protected example.

### Step C

Starting from the product of Step B following General procedure I Step B the appropriate example was obtained.

### General Procedure VII

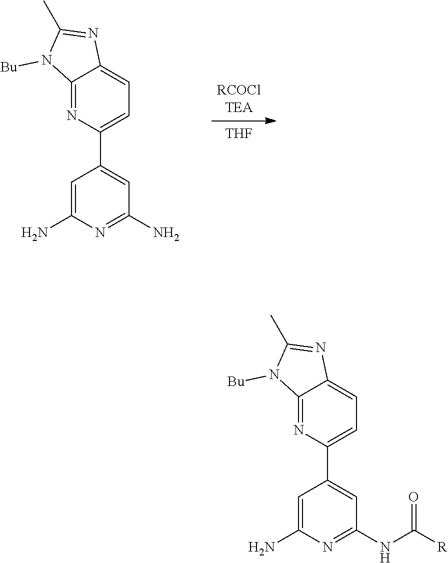

1.05 eq. of the appropriate acid chloride derivative was added dropwise at −78° C. to the solution of 1 eq. 4-(3-butyl-2-methyl-3H-imidazo[4,5-b]pyridin-5-yl)pyridine-2,6-diamine (Example 148) and 3 eq. triethylamine in THF (16 mL/mmol of Example 148). The resulting mixture was allowed to warm up to room temperature and stirred until no further conversion was observed. The volatiles were evaporated under reduced pressure and the crude product was purified via preparative reversed phase chromatography using 5 mM aqueous NH₄HCO₃ solution and MeCN as eluents to give the appropriate example.

### General Procedure VIII

### Step A

The mixture of 1 eq. 3-acetamino-2-fluoro-6-bromopyridine and 5 eq. of the appropriate amine in ethanol (2 M for the amine) was stirred at 50° C. until no further conversion was observed. Solvent and excess amine were removed under reduced pressure and the crude 3-acetamino-2-amino-6-bromopyridine derivative was used in the next step without further purification.

### Step B

The solution of the crude 3-acetamino-2-amino-6-bromopyridine derivative in acetic acid (1.2 mL/mmol of starting 3-acetamino-2-fluoro-6-bromopyridine) was heated at 120° C. until no further conversion was observed. The solvent was removed under reduced pressure, the residue was taken up in EtOAc, the organic phase was washed with 10/o K₂CO₃, brine, it was dried over anhydrous MgSO₄ and evaporated to dryness under reduced pressure. The crude product was purified by column chromatography using heptane and EtOAc as eluents to give the appropriate 3-substituted 5-bromo-2-methyl-imidazo[4,5-b]pyridine derivative.

### General Procedure IX

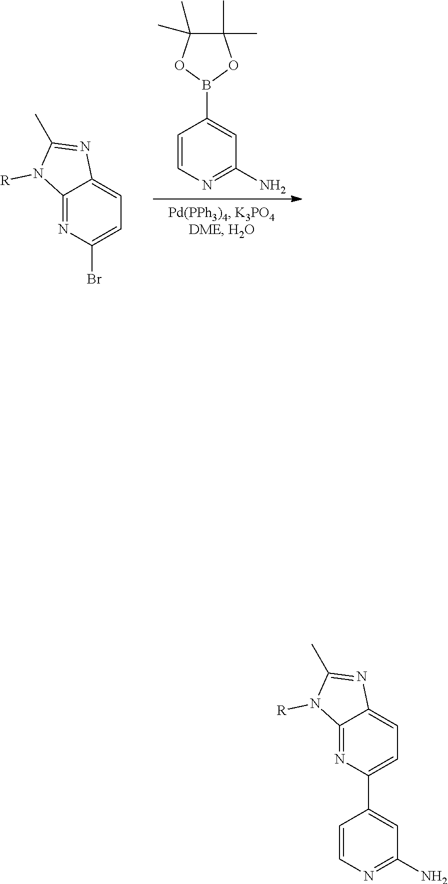

To the solution of 1 eq. of the appropriate aryl halide derivative in 1,2-dimethoxyethane-water 7:1 (8 mL/mmol), 1.1 eq 4-(4,4,5,5-tetramethyl-1,3,2-dioxaborolan-2-yl)pyridin-2-amine, 3 eq. K₃PO₄, 0.05 eq. Pd(OAc)₂, and 0.1 eq. ⁿBuPAd₂ were added, and the mixture was stirred at 90° C. under argon atmosphere until no further conversion was observed. The mixture was filtered through a pad of celite, the filtrate was concentrated under reduced pressure and purified via preparative reversed phase chromatography using 5 mM aqueous NH₄HCO₃ solution and MeCN as eluents to give the appropriate example.

### General Procedure X

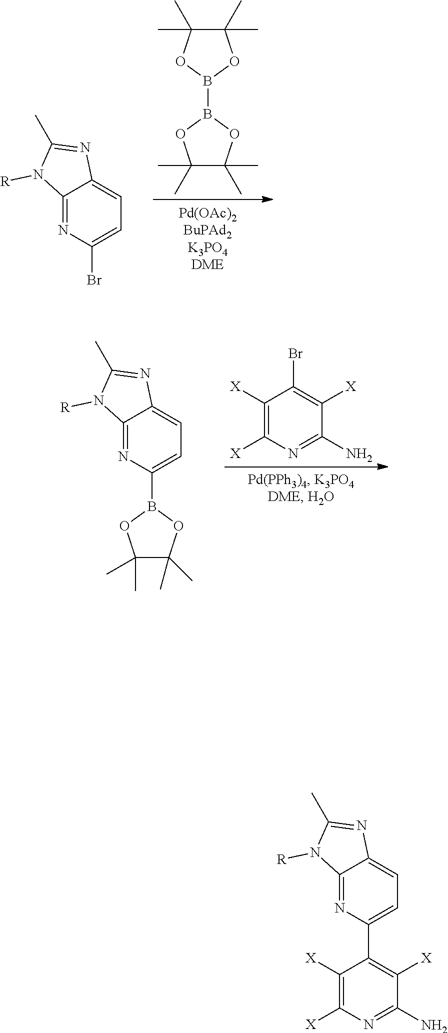

### Step A

A mixture of 1 eq. the appropriate 3-substituted 5-bromo-2-methyl-imidazo[4,5-b]pyridine derivative, 2.4 eq. 4,4,5,5-tetramethyl-2-(4,4,5,5-tetramethyl-1,3,2-dioxaborolan-2-yl)-1,3,2-dioxaborolane, 0.1 eq. Pd(OAc)₂, 0.2 eq. bis(1-adamantyl)-butyl-phosphane, and 3 eq. K₃PO₄ was dispensed in 1,2-dimethoxyethane (0.25 M solution for the imidazopyridine derivative) and the resulting mixture was stirred at 90° C. under nitrogen atmosphere until no further conversion was observed. The reaction mixture was filtered through celite and the celite was washed with 1,2-dichloroethane. Organic layers were combined, dried over MgSO₄, the solvent was removed under reduced pressure and the crude product was purified by flash chromatography using dichloromethane and methanol as eluents to give appropriate 3-substituted-2-methyl-5-(4,4,5,5-tetramethyl-1,3,2-dioxaborolan-2-yl)imidazo[4,5-b]pyridine.

### Step B

A mixture of 1 eq. of the 3-substituted-2-methyl-5-(4,4,5,5-tetramethyl-1,3,2-dioxaborolan-2-yl)imidazo[4,5-b]pyridine, 1.05 eq. of the 4-bromopyridine derivative, 0.1 eq. Pd(OAc)₂, 0.2 eq. bis(1-adamantyl)-butyl-phosphane and 4 eq. K₃PO₄ was dispensed in 1,2-dimethoxyethane (0.17 M solution for the imidazopyridine derivative). The reaction mixture was stirred at 90° C. under nitrogen atmosphere until no further conversion was observed. The reaction mixture was filtered through celite and the celite was washed with 1,2-dichloroethane. Organic layers were combined, dried over MgSO₄, the solvent was removed under reduced pressure and the crude product was purified by flash chromatography using dichloromethane and methanol as eluents to give the expected product.

### Preparation 1: tert-butyl N-[6-(tert-butoxycarbonylamino)-4-(4,4,5,5-tetramethyl-1,3,2-dioxaborolan-2-yl)-2-pyridyl]carbamate

109.7 g (4-bromo-6-tert-butoxycarbonylamino-pyridin-2-yl)-carbamicacid tert-butyl ester (283 mmol), prepared following J. Org. Chem. 2004, 69, 543-548, 107.7 g bis(pinacolato)diboron (424 mmol), 0.29 g Pd(OAc)₂ (1.27 mmol), 0.70 g 1,1′-bis(diphenylphosphino)ferrocene (1.27 mmol) and 83.2 g KOAc (848 mmol) were added to 1100 mL previously degassed 1,4-dioxane, and the mixture was stirred at 80° C. under argon atmosphere until no further conversion was observed. Then the reaction mixture was filtered; the solid was washed with dioxane. 5.5 g charcoal was added to the filtrate, and it was-stirred for 2 minutes at reflux temperature. The mixture was filtered, washed with warm 1,4-dioxane and the volatiles were evaporated under reduced pressure. The residue was crystallised from tert-butyl-methyl-ether to give Preparation 1 as a white crystalline solid.

¹H NMR (500 MHz, CDCl₃) δ: 8.16 (brs, 2H), 7.92 (s, 2H), 1.54 (s, 18H), 1.34 (s, 12H).

### Preparation 2a tert-butyl N-[6-(tert-butoxycarbonylamino)-4-[6-chloro-5-(acetylamino)-2-pyridyl]-2-pyridyl]carbamate

### Step A: N-(6-bromo-2-chloro-3-pyridyl)acetamide

31.4 g 6-bromo-2-chloro-pyridin-3-amine (151.3 mmol) was dissolved in 200 ml glacial acetic acid, 15 mL acetic anhydride (158.9 mmol) was added to this solution dropwise and the reaction mixture was stirred at room temperature until no further conversion was observed. The reaction mixture was concentrated under reduced pressure. The residue was dissolved in ethyl acetate and the organic phase was washed with 10% aqueous K₂CO₃ and brine. Following drying over Na₂SO₄ removal of the solvents under reduced pressure gave N-(6-bromo-2-chloro-3-pyridyl)acetamide.

HPLC-MS: (M−H)=247.0; 249.0

### Step B: tert-butyl N-[6-(tert-butoxycarbonylamino)-4-[6-chloro-5-(acetylamino)-2-pyridyl]-2-pyridyl]carbamate

13.1 g N-(6-bromo-2-chloro-3-pyridyl)acetamide (52.5 mmol), 24.0 g. tert-butyl N-[6-(tert-butoxycarbonylamino)-4-(4,4,5,5-tetramethyl-1,3,2-dioxaborolan-2-yl)-2-pyridyl]carbamate (Preparation 1) (55.13 mmol) and 33.4 g K₃PO₄ (157.3 mmol) were dissolved in 1,2-dimethoxyethane-water 4:1 (250 mL). Then 304 mg tetrakis(triphenylphosphine)palladium(0) (0.26 mmol) was added and the mixture was heated under nitrogen at 90° C. until no further conversion was observed. Then the mixture was diluted with 250 mL water and extracted with EtOAc. The organic layer was dried over Na₂SO₄, the volatiles were removed under reduced pressure and the residue was recrystallized from EtOAc to obtain tert-butyl N-[6-(tert-butoxycarbonylamino)-4-[6-chloro-5-(acetylamino)-2-pyridyl]-2-pyridyl]carbamate.

¹H NMR (500 MHz, DMSO-d6) δ: 9.80 (s, 1H), 9.51 (s, 2H), 8.40 (d, 1H), 8.00 (s, 2H), 7.92 (d, 1H), 2.18 (s, 3H), 1.49 (s, 18H).

### Preparation 2b tert-butyl N-[6-(tert-butoxycarbonylamino)-4-[6-chloro-5-(pentanoylamino)-2-pyridyl]-2-pyridyl]carbamate

### Step A: N-(6-bromo-2-chloro-3-pyridyl)pentanamide

3.0 g 6-bromo-2-chloro-pyridin-3-amine (14.5 mmol) and 2.4 mL triethylamine (17.4 mmol) were dissolved in 60 mL DCM. The solution was cooled to 0° C. and 2.1 ml pentanoyl chloride (17.4 mmol) was added dropwise over 30 minutes. On completion of the addition the reaction mixture was allowed to warm up to room temperature where it was stirred until no further conversion was observed. The reaction mixture was diluted with ethyl acetate and washed with water. The organic layer was washed with brine, dried over MgSO₄, filtered and concentrated under reduced pressure. The residue was purified via flash chromatography using DCM as eluent to give N-(6-bromo-2-chloro-3-pyridyl)pentanamide, a pale pink solid.

HPLC-MS: (M−H)=289.0; 291.0

### Step B: tert-butyl N-[6-(tert-butoxycarbonylamino)-4-[6-chloro-5-(pentanoylamino)-2-pyridyl]-2-pyridyl]carbamate

Starting from N-(6-bromo-2-chloro-3-pyridyl)pentanamide following Step B of Preparation 2a, tert-butyl N-[6-(tert-butoxycarbonylamino)-4-[6-chloro-5-(pentanoylamino)-2-pyridyl]-2-pyridyl]carbamate was obtained.

¹H NMR (500 MHz, DMSO-d6) δ: 9.70 (s, 1H), 9.45 (s, 2H), 8.37 (d, 1H), 8.00 (s, 2H), 7.91 (d, 1H), 2.47 (t, 2H), 1.6 (m, 2H), 1.49 (s, 18H), 1.36 (m, 2H), 0.91 (t, 3H).

### Preparation 2c tert-butyl N-[6-(tert-butoxycarbonylamino)-4-[6-chloro-5-(propanoylamino)-2-pyridyl]-2-pyridyl]carbamate

### Step A: N-(6-bromo-2-chloro-3-pyridyl)propanamide

3.0 g 6-bromo-2-chloro-pyridin-3-amine (14.5 mmol) and 2.4 mL triethylamine (17.4 mmol), were dissolved in 60 ml DCM. The solution was cooled to 0° C. and 1.5 ml propanoyl chloride was added dropwise over 30 minutes. On completion of the addition the reaction mixture was allowed to warm up to room temperature where it was stirred until no further conversion was observed. The reaction mixture was diluted with ethyl acetate and washed with water. The organic layer was washed with brine, dried over MgSO₄, filtered and concentrated under reduced pressure. The residue was purified via flash chromatography using DCM as eluent to give N-(6-bromo-2-chloro-3-pyridyl)propanamide.

HPLC-MS: (M−H)=261.0; 263.0

### Step B: tert-butyl N-[6-(tert-butoxycarbonylamino)-4-[6-chloro-5-(propanoylamino)-2-pyridyl]-2-pyridyl]carbamate

Starting from N-(6-bromo-2-chloro-3-pyridyl)propanamide following Step B of Preparation 2a, tert-butyl N-[6-(tert-butoxycarbonylamino)-4-[6-chloro-5-(propanoylamino)-2-pyridyl]-2-pyridyl]carbamate was obtained.

¹H NMR (500 MHz, DMSO-d6) δ: 9.68 (s, 1H), 9.45 (s, 2H), 8.40 (d, 1H), 8.00 (s, 2H), 7.91 (d, 1H), 2.48 (q, 2H), 1.49 (s, 18H), 1.11 (t, 3H).

### Preparation 2d tert-butyl N-[6-(tert-butoxycarbonylamino)-4-[6-chloro-5-(butanoylamino)-2-pyridyl]-2-pyridyl]carbamate

### Step A: N-(6-bromo-2-chloro-3-pyridyl)butanamide

3 g 6-bromo-2-chloro-pyridin-3-amine (14.46 mmol) and 2.4 ml triethylamine (17.35 mmol, 1.2 eq.), were dissolved in 60 ml DCM. This solution was cooled down to 0° C. and 1.8 ml (17.35 mmol, 1.2 eq.) butanoyl chloride was added dropwise over 30 minutes. On completion of the addition the reaction mixture was allowed to warm up to room temperature where it was stirred until no further conversion was observed. The reaction mixture was diluted with ethyl acetate and washed with water. The organic layer was washed with brine, dried over MgSO₄, filtered and concentrated under reduced pressure. The residue was purified via flash chromatography using DCM as eluent to give N-(6-bromo-2-chloro-3-pyridyl)butanamide.

HPLC-MS: (M−H)=275.0; 277.0

### Step B: tert-butyl N-[6-(tert-butoxycarbonylamino)-4-[6-chloro-5-(butanoylamino)-2-pyridyl]-2-pyridyl]carbamate

Starting from N-(6-bromo-2-chloro-3-pyridyl)butanamide following Step B of Preparation 2a, tert-butyl N-[6-(tert-butoxycarbonylamino)-4-[6-chloro-5-(butiroylamino)-2-pyridyl]-2-pyridyl]carbamate was obtained.

¹H NMR (500 MHz, DMSO-d6) δ: 9.70 (s, 1H), 9.46 (s, 2H), 8.37 (d, 1H), 8.00 (s, 2H), 7.91 (d, 1H), 2.45 (t, 2H), 1.64 (m, 2H), 1.49 (s, 18H), 0.95 (t, 3H).

### Preparation 3a tert-butyl N-[6-(tert-butoxycarbonylamino)-4-[3-(2-hydroxyethyl)-2-methyl-imidazo[4,5-b]pyridin-5-yl]-2-pyridyl]carbamate

1 eq. 5-chloro-3-(2-hydroxyethyl)-2-methyl-imidazo[4,5-b]pyridine, 1.1 eq. tert-butyl N-[6-(tert-butoxycarbonylamino)-4-(4,4,5,5-tetramethyl-1,3,2-dioxaborolan-2-yl)-2-pyridyl]carbamate (Preparation 1), 0.1 eq. Pd(OAc)₂, 0.2 eq. PBuAd₂, and 3.0 eq. K₂CO₃ were suspended in DME (0.2 M) and the mixture was stirred under nitrogen at 100° C. in a microwave reactor until no further conversion was observed. The volatiles were removed under reduced pressure and the crude product was purified via flash chromatography using dichloromethane and methanolic ammonia as eluents to give tert-butyl N-[6-(tert-butoxycarbonylamino)-4-[3-(2-hydroxyethyl)-2-methyl-imidazo[4,5-b]pyridin-5-yl]-2-pyridyl]carbamate as a white solid.

¹H NMR (400 MHz, DMSO-d6) δ: 9.34 (s, 2H), 8.03 (d, 1H), 8.02 (s, 2H), 7.68 (d, 1H), 4.97 (t, 1H), 4.34 (t, 2H), 3.81 (q, 2H), 2.63 (s, 3H), 1.49 (s, 18H).

### Preparation 3b tert-butyl N-[6-(tert-butoxycarbonylamino)-4-[3-(2-hydroxypropyl)-2-methyl-imidazo[4,5-b]pyridin-5-yl]-2-pyridyl]carbamate

### Step A: 3-amino-2-fluoro-6-bromopyridine

1 eq. 3-amino-2-fluoropyridine was dissolved in DCM (0.6 M solution), 1.05 eq. N-bromosuccinimide was added and the reaction mixture was stirred at room temperature until no further conversion was observed. Water was added, the organic phase was separated, dried over anhydrous MgSO₄ and concentrated under reduced pressure to give 3-amino-2-fluoro-6-bromopyridine.

¹H NMR (400 MHz, DMSO-d6) δ: 7.22 (dd, 1H), 7.11 (dd, 1H), 5.62 (brs, 2H).

### Step B: 3-acetamino-2 fluoro-6-bromopyridine

To a solution of 1 eq. 3-amino-2-fluoro-6-bromopyridine in acetic acid (0.9 M) 1.05 eq. acetic anhydride was added and the reaction mixture was stirred at room temperature until no further conversion was observed. The solvents were removed reduced pressure, the crude product was dissolved in DCM and washed with 10% K₂CO₃. The organic layer was dried over anhydrous MgSO₄, and concentrated under reduced pressure to give 3-acetamino-2-fluoro-6-bromopyridine.

¹H NMR (400 MHz, DMSO-d6) δ: 10.03 (brs, 1H), 8.42 (dd, 1H), 7.56 (d, 1H), 2.11 (s, 3H).

### Step C: 3-acetamino-2-(2-hydroxypropylamino)-6-bromopyridine

The mixture of 1 eq. 3-acetamino-2-fluoro-6-bromopyridine, 2.2 eq. 1-aminopropan-2-ol, and triethylamine (0.27 mL/mmol of the fluoropyridine) was stirred at 60° C. until no further conversion was observed. Solvent and excess amine were removed under reduced pressure and the crude 3-acetamino-2-(2-hydroxypropylamino)-6-bromopyridine was used in the next step without purification.

MS: (M+H)⁺=288.2

### Step D: 5-bromo-3-(2-hydroxypropyl)-2-methyl-imidazo[4,5-b]pyridine

The solution of 1 eq. 3-acetamino-2-(2-hydroxypropylamino)-6-bromopyridine in acetic acid (13.6 mL/g of the crude amide) was heated at 130° C. until no further conversion was observed. The solvent was removed under reduced pressure, the residue was taken up in methanol:water (5:1, 7 mL/g of residue) containing LiOH*H₂O (0.27 g/g of residue) and the mixture was stirred at ambient temperature for 2 hours than poured into water. The precipitate was filtered off, washed with water and dried to give 5-bromo-3-(2-hydroxypropyl)-2-methyl-imidazo[4,5-b]pyridine.

¹H NMR (400 MHz, DMSO-d6) δ: 7.87 (d, 1H), 7.36 (d, 1H), 4.97 (d, 1H), 4.19-3.94 (m, 3H), 2.57 (s, 3H), 1.21 (d, 3H).

### Step E

1 eq. S-bromo-3-(2-hydroxypropyl)-2-methyl-imidazo[4,5-b]pyridine, 1.0 eq. tert-butyl N-[6-(tert-butoxycarbonylamino)-4-(4,4,5,5-tetramethyl-1,3,2-dioxaborolan-2-yl)-2-pyridyl]carbamate (Preparation 1), 0.05 eq. tetrakis(triphenylphosphine)palladium(0), and 3 eq. K₃PO₄ were suspended in DME (5 mL/mmol for the bromo compound) and the mixture was stirred under nitrogen at 100° C. in a microwave reactor until no further conversion was observed. The volatiles were removed under reduced pressure and the crude product was purified via flash chromatography using dichloromethane and methanolic ammonia as eluents to give tert-butyl N-[6-(tert-butoxycarbonylamino)-4-[3-(2-hydroxypropyl)-2-methyl-imidazo[4,5-b]pyridin-5-yl]-2-pyridyl]carbamate as a white solid.

¹H NMR (500 MHz, DMSO-d6) δ: 9.42 (s, 2H), 8.10 (s, 2H), 8.03 (d, 11H), 7.70 (d, 1H), 5.03 (d, 1H), 4.28 (dd, 1H), 4.18 (m, 1H), 4.10 (dd, 1H), 2.64 (s, 3H), 1.50 (s, 18H), 1.19 (d, 3H).

### Preparation 4 6-chloro-N²-methyl-pyridine-2,3-diamine

### Step A: 6-chloro-N-methyl-3-nitro-pyridin-2-amine

3.86 g 2,6-dichloro-3-nitro-pyridine (20 mmol) was dissolved in 80 ml DCM, 6.9 g K₂CO (50 mmol, 2.5 eq.) was added and the reaction mixture was cooled down to −20° C. At this temperature 3.1 ml of methylamine (33% solution in ethanol, 28.6 mmol, 1.43 eq.) was added dropwise then cooling was stopped and the reaction mixture was allowed to warm up to ambient temperature where it was stirred until no further conversion was observed. The reaction mixture was filtered, the filtrate was washed with water, the organic layer was dried on MgSO₄ then concentrated under reduced pressure to give 6-chloro-N-methyl-3-nitro-pyridin-2-amine as a solid.

¹H NMR (500 MHz, DMSO-d6) δ: 8.72 (d, 1H), 8.42 (d, 1H), 6.77 (d, 1H), 3.08 (d, 3H).

### Step B: 6-chloro-2-methylamino-3-aminopyridine

3.0 g 6-chloro-N-methyl-3-nitro-pyridin-2-amine (16 mmol) was dissolved in the mixture of 30 ml ethanol and 15 ml water then 4.47 g iron powder (80 mmol, 5 eq.) was added. To this mixture 1.2 ml glacial acetic acid was added dropwise then the reaction mixture was refluxed until no further conversion was observed. The reaction mixture was filtered, the filtrate was concentrated under reduced pressure and the residue was purified via flash chromatography using DCM as eluent to give 6-chloro-2-methylamino-3-aminopyridine. MS (M+H)=158.2

### Preparation 4-(3-butyl-2-methyl-imidazo[4,5-b]pyridin-5-yl)-N²-triphenylmethyl-pyridine-2,6-diamine

8.35 mL triethylamine (6.07 g, 60.0 mmol) and 8.36 g triphenylmethyl chloride (30.0 mmol) were added at room temperature to a stirred solution of 2.96 g 4-(3-butyl-2-methyl-3H-imidazo[4,5-b]pyridin-5-yl)pyridine-2,6-diamine (Example 148) (10.0 mmol) in 100 mL THF and the mixture was stirred until no further conversion was observed. The volatiles were evaporated under reduced pressure and the crude product was purified via reversed phase flash chromatography using water and MeCN as eluents to obtain 4-(3-butyl-2-methyl-imidazo[4,5-b]pyridin-5-yl)-N²,N⁶-di(triphenylmethyl)-pyridine-2,6-diamine as an intermediate. This intermediate was dissolved in 400 mL methanol, 20 mL TFA was added at room temperature and the mixture was stirred at room temperature until the total amount of the bis-triphenylmethylated intermediate was converted to the desired product. Then 16.0 g NH₄HCO₃ (202.4 mmol) was added with stirring and the formed precipitate was removed by filtration to obtain the crude product, which was recrystallized from methanol to give 4-(3-butyl-2-methyl-imidazo[4,5-b]pyridin-5-yl)-N-triphenylmethyl-pyridine-2,6-diamine (Preparation 5).

¹H NMR (500 MHz, CDCl₃) δ: 7.84 (d, 1H), 7.42-7.35 (m, 6H), 7.33-7.25 (m, 6H), 7.23-7.16 (m, 3H), 7.20 (d, 1H), 6.33 (s, 1H), 6.26 (brs, 1H), 6.03 (s, 1H), 5.45 (brs, 2H), 4.12 (t, 2H), 2.58 (s, 3H), 1.64 (m, 2H), 1.16 (m, 2H), 0.85 (t, 3H).

### Preparation 6a 5-bromo-3-butyl-2-methyl-imidazo[4,5-b]pyridine

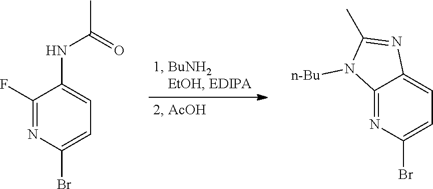

Following General procedure VIII and using butylamine as the appropriate amine derivative 5-bromo-3-butyl-2-methyl-imidazo[4,5-b]pyridine was obtained.

¹H NMR (500 MHz, DMSO-d6) δ: 7.89 (d, 1H), 7.38 (d, 1H), 4.18 (t, 2H), 2.58 (s, 3H), 1.71 (quint, 2H), 1.38-1.22 (m, 2H), 0.91 (t, 3H).

### Preparation 6b 5-bromo-3-(2-cyclohexylethyl)-2-methyl-imidazo[4,5-b]pyridine

Following General procedure VIII and using (2-aminoethyl)-cyclohexane as the appropriate amine derivative 5-bromo-3-(2-cyclohexylethyl)-2-methyl-imidazo[4,5-b]pyridine was obtained.

¹H NMR (500 MHz, DMSO-d6) δ: 7.88 (d, 1H), 7.38 (d, 1H), 4.19 (t, 2H), 2.57 (s, 3H), 1.79 (d, 2H), 1.66 (d, 2H), 1.62-1.57 (m, 1H), 1.59 (q, 2H), 1.29-1.08 (m, 4H), 0.94 (q, 2H).

### Preparation 6c 5-bromo-3-(cyclopropylmethyl)-2-methyl-imidazo[4,5-b]pyridine

Following General procedure VIII and using cyclopropyl-methylamine as the appropriate amine derivative 5-bromo-3-(cyclopropylmethyl)-2-methyl-imidazo[4,5-b]pyridine was obtained.

¹H NMR (400 MHz, DMSO-d6) δ: 7.77 (d, 1H), 7.31 (d, 1H), 4.10 (d, 2H), 2.66 (s, 3H), m 1.33-1.19 (m, 1H), 0.64-0.43 (m, 4H).

### Preparation 6d 5-bromo-3-but-3-enyl-2-methyl-imidazo[4,5-b]pyridine

Following General procedure VIII and using 1-amino-3-butene as the appropriate amine derivative 5-bromo-3-but-3-enyl-2-methyl-imidazo[4,5-b]pyridine was obtained.

¹H NMR (500 MHz, DMSO-d6) δ: 7.88 (d, 1H), 7.38 (d, 1H), 5.86-5.71 (m, 1H), 5.01-4.94 (m, 2H), 4.26 (t, 2H), 2.57 (s, 3H), 2.52 (q, 2H).

### Preparation 6e 5-bromo-3-(3,3-difluorocyclobutyl)-2-methyl-imidazo[4,5-b]pyridine

Following General procedure VIII and using 3,3-difluoro-cyclobutanamine as the appropriate amine derivative 5-bromo-3-(3,3-difluorocyclobutyl)-2-methyl-imidazo[4,5-b]pyridine was obtained.

¹H NMR (500 MHz, DMSO-d6) δ: 7.91 (d, 1H), 7.42 (d, 1H), 5.05-4.92 (m, 1H), 3.88-3.69 (m, 2H), 3.22-3.09 (m, 2H), 2.60 (s, 3H).

### Preparation 6f 5-bromo-3-cyclopropyl-2-methyl-imidazo[4,5-b]pyridine

Following General procedure VIII and using cyclopropylamine as the appropriate amine derivative 5-bromo-3-cyclopropyl-2-methyl-imidazo[4,5-b]pyridine was obtained.

¹H NMR (500 MHz, DMSO-d6) δ: 7.86 (d, 1H), 7.37 (d, 1H), 3.33-3.28 (m, 1H), 2.60 (s, 3H), 1.18-1.11 (m, 4H).

### Example 1 4-(3-cyclopentyl-2-methyl-3H-imidazo[4,5-b]pyridin-5-yl)pyridine-2,6-diamine

Starting from 6-chloro-3-cyclopentyl-2-methyl-imidazo[4,5-b]pyridine as the appropriate halide and following General procedure I Example 1 was obtained. HRMS (TOF, ESI) m/z: Calcd for C₁₇H₂₀N₆ 308.1749, Found: 309.1821 [M+H]⁺.

### Example 2 4-(2-methyl-3-propyl-3H-imidazo[4,5-b]pyridin-5-yl)pyridine-2,6-diamine

Starting from 3-propyl-6-chloro-2-methyl-imidazo[4,5-b]pyridine as the appropriate halide and following General procedure I Example 2 was obtained. HRMS (TOF, ESI) m/z: Calcd for C₁₅H₁₈N₆ 282.1593, Found: 283.1662 [M+H]⁺.

### Example 3 2-[5-(2,6-diaminopyridin-4-yl)-2-methyl-3H-imidazo[4,5-b]pyridin-3-yl]ethanol

Starting from 6-chloro-3-(2-hydroxyethyl)-2-methyl-imidazo[4,5-b]pyridine as the appropriate halide and following General procedure I Example 3 was obtained. HRMS (TOF, ESI) m/z: Calcd for C₁₄H₁₆N₆O 284.1386, Found: 285.1473 [M+H]⁺.

### Example 4 4-(2,3-dimethyl-3H-imidazo[4,5-b]pyridin-5-yl)pyridine-2,6-diamine

Starting from 6-chloro-2,3-dimethyl-imidazo[4,5-b]pyridine as the appropriate halide and following General procedure 1 Example 4 was obtained. HRMS (TOF, ESI) m/z: Calcd for C₁₃H₁₄N₆ 254.1280, Found: 255.1361 [M+H]⁺.

### Example 5 4-[2-methyl-3-(pyridin-4-ylmethyl)-3H-imidazo[4,5-b]pyridin-5-yl]pyridine-2,6-diamine

Starting from 6-chloro-3-(4-pyridylmethyl)-2-methyl-imidazo[4,5-b]pyridine as the appropriate halide and following General procedure I Example 5 was obtained. HRMS (TOF, ESI) m/z: Calcd for C₁₈H₁₇N₇ 331.1545, Found: 332.1623 [M+H]⁺.

### Example 6 4-[2-methyl-3-(pyridin-2-ylmethyl)-3H-imidazo[4,5-b]pyridin-5-yl]pyridine-2,6-diamine

Starting from 6-chloro-3-(2-pyridylmethyl)-2-methyl-imidazo[4,5-b]pyridine as the appropriate halide and following General procedure I Example 6 was obtained. HRMS (TOF, ESI) m/z: Calcd for C₁₈H₁₇N₇ 331.1545, Found: 332.1625 [M+H]⁺.

### Example 7 4-[2-methyl-3-(pyridin-3-ylmethyl)-3H-imidazo[4,5-b]pyridin-5-yl]pyridine-2,6-diamine

Starting from 6-chloro-3-(3-pyridylmethyl)-2-methyl-imidazo[4,5-b]pyridine as the appropriate halide and following General procedure I Example 7 was obtained. HRMS (TOF, ESI) m/z: Calcd for C₁₈H₁₇N₇ 331.1545, Found: 332.1625 [M+H]⁺.

### Example 8 4-(3-benzyl-2-methyl-3H-imidazo[4,5-b]pyridin-5-yl)pyridine-2,6-diamine

Starting from 3-benzyl-6-chloro-2-methyl-imidazo[4,5-b]pyridine as the appropriate halide and following General procedure I Example 8 was obtained. HRMS (TOF, ESI) m/z: Calcd for C₁₉H₁₈N₆ 330.1593, Found: 331.1673 [M+H]⁺.

### Example 9 4-(3-cyclopropyl-2-methyl-3H-imidazo[4,5-b]pyridin-5-yl)pyridine-2,6-diamine

Starting from Preparation 2a following General procedure II and using cyclopropylamine as the appropriate amine Example 9 was obtained. HRMS (TOF, ESI) m/z: Calcd for C₁₅H₁₆N₆ 280.1436, Found: 281.1518 [M+H]⁺.

### Example 10 4-[3-(4-fluorobenzyl)-2-methyl-3H-imidazo[4,5-b]pyridin-5-yl]pyridine-2,6-diamine

Starting from Preparation 2a following General procedure II and using 4-fluorobenzylamine as the appropriate amine Example 10 was obtained. HRMS (TOF, ESI) m/z: Calcd for C₁₉H₁₇N₆F 348.1499, Found: 349.1565 [M+H]⁺.

### Example 11 4-[3-(cyclopropylmethyl)-2-methyl-3H-imidazo[4,5-b]pyridin-5-yl]pyridine-2,6-diamine

Starting from Preparation 2a following General procedure II and using cyclopropylmethylamine as the appropriate amine Example 11 was obtained. HRMS (TOF, ESI) m/z: Calcd for C₁₆H₁₈N₆ 294.1593, Found: 295.1665 [M+H]⁺.

### Example 12 4-[3-(2,3-dihydro-1H-inden-2-yl)-2-methyl-3H-imidazo[4,5-b]pyridin-5-yl]pyridine-2,6-diamine

Starting from Preparation 2a following General procedure II and using 2,3-dihydro-1H-inden-2-amine as the appropriate amine Example 12 was obtained. HRMS (TOF, ESI) m/z: Calcd for C₂₁H₂₀N₆ 356.1749, Found: 357.1822 [M+H]⁺.

### Example 13 1-{3-[5-(2,6-diaminopyridin-4-yl)-2-methyl-3H-imidazo[4,5-b]pyridin-3-yl]propyl}pyrrolidin-2-one

Starting from Preparation 2a following General procedure II and using 1-(3-aminopropyl)pyrrolidin-2-one as the appropriate amine Example 13 was obtained. HRMS (TOF, ESI) m/z: Calcd for C₁₉H₂₃N₇O 365.1964, Found: 366.2035 [M+H]⁺.

### Example 14 4-[3-(but-3-en-1-yl)-2-methyl-3H-imidazo[4,5-b]pyridin-5-yl]pyridine-2,6-diamine

Starting from Preparation 2a following General procedure II and using 4-amino-1-butene as the appropriate amine Example 14 was obtained. HRMS (TOF, ESI) m/z: Calcd for C₁₆H₁₈N₆ 294.1593, Found: 295.1672 [M+H]⁺.

### Example 15 4-[3-(2-cyclohexylethyl)-2-methyl-3H-imidazo[4,5-b]pyridin-5-yl]pyridine-2,6-diamine

Starting from Preparation 2a following General procedure 11 and using (2-aminoethyl)-cyclohexane as the appropriate amine Example 15 was obtained. HRMS (TOF, ESI) m/z: Calcd for C₂₀H₂₆N₆ 350.2219, Found: 351.2298 [M+H]⁺.

### Example 16 4-[2-methyl-3-[(1S,2S,3S,5R)-2,6,6-trimethylbicyclo[3.1.1]hept-3-yl]-3H-imidazo[4,5-b]pyridin-5-yl]pyridine-2,6-diamine

Starting from Preparation 2a following General procedure II and using (1S,2S,3S,5R)-2,6,6-trimethylbicyclo[3.1.1]heptan-3-amine as the appropriate amine Example 16 was obtained. HRMS (TOF, ESI) m/z: Calcd for C₂₂H₂₈N₆ 376.2375, Found: 377.2456 [M+H]⁺.

### Example 17 4-[3-(2-cyclopropylethyl)-2-methyl-3H-imidazo[4,5-b]pyridin-5-yl]pyridine-2,6-diamine

Starting from Preparation 2a following General procedure II and using 2-cyclopropylethanamine as the appropriate amine Example 17 was obtained. HRMS (TOF, ESI) m/z: Calcd for C₁₇H₂₀N₆ 308.1749, Found: 309.1828 [M+H]⁺.

### Example 18 4-[3-(2-ethylbutyl)-2-methyl-3H-imidazo[4,5-b]pyridin-5-yl]pyridine-2,6-diamine

Starting from Preparation 2a following General procedure 11 and using 2-ethylbutan-1-amine as the appropriate amine Example 18 was obtained. HRMS (TOF, ESI) m/z: Calcd for C₁₈H₂₄N₆ 324.2062, Found: 325.2139 [M+H]⁺.

### Example 19 4-{3-[2-(furan-2-yl)ethyl]-2-methyl-3H-imidazo[4,5-b]pyridin-5-yl}pyridine-2,6-diamine

Starting from Preparation 2a following General procedure II and using 2-(2-furyl)ethanamine as the appropriate amine Example 19 was obtained. HRMS (TOF, ESI) m/z: Calcd for C₁₈H₁₈N₆O 334.1542, Found: 335.1618 [M+H]⁺.

### Example 20 4-[2-methyl-3-(thiophen-2-ylmethyl)-3H-imidazo[4,5-b]pyridin-5-yl]pyridine-2,6-diamine

Starting from Preparation 2a following General procedure II and using 2-thienylmethanamine as the appropriate amine Example 20 was obtained. HRMS (TOF, ESI) m/z: Calcd for C₁₇H₁₆N₆S 336.1157, Found: 337.1224 [M+H]⁺.

### Example 21 4-{2-methyl-3-[2-(1-phenyl-1H-pyrazol-4-yl)ethyl]-3H-imidazo[4,5-b]pyridin-5-yl}pyridine-2,6-diamine

Starting from Preparation 2a following General procedure II and using 2-(1-phenylpyrazol-4-yl)ethanamine as the appropriate amine Example 21 was obtained. HRMS (TOF, ESI) m/z: Calcd for C₂₃H₂₂N₈ 410.1967, Found: 411.2038 [M+H]⁺.

### Example 22 4-{2-methyl-3-tricyclo[3.3.1.1^{3,7}]dec-1-ylmethyl-3H-imidazo[4,5-b]pyridin-5-yl}pyridine-2,6-diamine

Starting from Preparation 2a following General procedure II and using 1-adamantylmethanamine as the appropriate amine Example 22 was obtained. HRMS (TOF, ESI) m/z: Calcd for C₂₃H₂₈N₆ 388.2375, Found: 389.2542 [M+H]⁺.

### Example 23 4-(3-cyclobutyl-2-methyl-3H-imidazo[4,5-b]pyridin-5-yl)pyridine-2,6-diamine

Starting from Preparation 2a following General procedure II and using cyclobutylamine as the appropriate amine Example 23 was obtained. HRMS (TOF, ESI) m/z: Calcd for C₁₆H₁₈N₆ 294.1593, Found: 295.1665 [M+H]⁺.

### Example 24 N-(4-{2-[5-(2,6-diaminopyridin-4-yl)-2-methyl-3H-imidazo[4,5-b]pyridin-3-yl]ethyl}phenyl)acetamide

Starting from Preparation 2a following General procedure II and using N-[4-(2-aminoethyl)phenyl]acetamide as the appropriate amine Example 24 was obtained. HRMS (TOF, ESI) m/z: Calcd for C₂₂H₂₃N₇O 401.1964, Found: 402.2039 [M+H]⁺.

### Example 25 4-(3-tert-butyl-2-methyl-3H-imidazo[4,5-b]pyridin-5-yl)pyridine-2,6-diamine

Starting from Preparation 2a following General procedure II and using tert-butylamine as the appropriate amine Example 25 was obtained. HRMS (TOF, ESI) m/z: Calcd for C₁₆H₂₀N₆ 296.1479, Found: 240.1125 [M+H—C₄H₈]⁺. Fragment ion formula: C₁₂H₁₂N₆ no molecular ion was detected due to extensive fragmentation.

### Example 26 4-{2-methyl-3-[2-(thiophen-2-yl)ethyl]-3H-imidazo[4,5-b]pyridin-5-yl}pyridine-2,6-diamine

Starting from Preparation 2a following General procedure II and using 2-thienylethaneamine as the appropriate amine Example 26 was obtained. HRMS (TOF, ESI) m/z: Calcd for C₁₈H₁₈N₆S 350.1314, Found: 351.1385 [M+H]⁺.

### Example 27 4-{2-methyl-3-[2-(naphthalen-1-yloxy)ethyl]-3H-imidazo[4,5-b]pyridin-5-yl}pyridine-2,6-diamine

Starting from Preparation 2a following General procedure II and using 2-(naphthalen-1-yloxy)ethanamine as the appropriate amine Example 27 was obtained. HRMS (TOF, ESI) m/z: Calcd for C₂₄H₂₂N₆O 410.1855, Found: 411.1923 [M+H]⁺.

### Example 28 4-[2-methyl-3-(2,2,2-trifluoroethyl)-3H-imidazo[4,5-b]pyridin-5-yl]pyridine-2,6-diamine

Starting from Preparation 2a following General procedure II and using 2,2,2-trifluoroethylamine as the appropriate amine Example 28 was obtained. HRMS (TOF, ESI) m/z: Calcd for C₁₄H₁₃N₆F₃ 322.1154, Found: 323.1238 [M+H]⁺.

### Example 29 4-{2-methyl-3-[2-(thiophen-3-yl)ethyl]-3H-imidazo[4,5-b]pyridin-5-yl}pyridine-2,6-diamine

Starting from Preparation 2a following General procedure II and using 3-thienylethaneamine as the appropriate amine Example 29 was obtained. HRMS (TOF, ESI) m/z: Calcd for C₁₈H₁₈N₆S 350.1314, Found: 351.1379 [M+H]⁺.

### Example 30 4-[3-(2,2-dimethylpropyl)-2-methyl-3H-imidazo[4,5-b]pyridin-5-yl]pyridine-2,6-diamine

Starting from Preparation 2a following General procedure II and using 2,2-dimethylpropan-1-amine as the appropriate amine Example 30 was obtained. HRMS (TOF, ESI) m/z: Calcd for C₁₇H₂₂N₆ 310.1906, Found: 311.2010 [M+H]⁺.

### Example 31 4-[2-methyl-3-(2-methylpropyl)-3H-imidazo[4,5-b]pyridin-5-yl]pyridine-2,6-diamine

Starting from Preparation 2a following General procedure 11 and using 2-methylpropan-1-amine as the appropriate amine Example 31 was obtained. HRMS (TOF, ESI) m/z: Calculated for C₁₆H₂₀N₆ 296.1749, Found: 297.1824 [M+H]⁺.

### Example 32 4-{2-methyl-3-[(I R)-1-(2-methylpyridin-4-yl)ethyl]-3H-imidazo[4,5-b]pyridin-5-yl}pyridine-2,6-diamine

Starting from Preparation 2a following General procedure II and using (1R)-1-(2-methyl-4-pyridyl)ethanamine as the appropriate amine Example 32 was obtained. HRMS (IT-TOF, ESI) m/z: Calculated for C₂₀H₂₁N₇ 359.1858, Found: 360.1934 [M+H]⁺.

### Example 33 4-[3-(butan-2-yl)-2-methyl-3H-imidazo[4,5-b]pyridin-5-yl]pyridine-2,6-diamine

Starting from Preparation 2a following General procedure II and using butan-2-amine as the appropriate amine Example 33 was obtained. HRMS (IT-TOF, ESI) m/z: Calculated for Cl₆H₂₀N₆ 296.1749, Found: 297.1826 [M+H]⁺.

### Example 34 4-[2-methyl-3-(2-methylbutyl)-3H-imidazo[4,5-b]pyridin-5-yl]pyridine-2,6-diamine

Starting from Preparation 2a following General procedure II and using 2-methylbutan-1-amine as the appropriate amine Example 34 was obtained. HRMS (IT-TOF, ESI) m/z: Calculated for C₁₇H₂₂N₆ 310.1906, Found: 311.1983 [M+H]⁺.

### Example 35 ethyl 4-[5-(2,6-diaminopyridin-4-yl)-2-methyl-3H-imidazo[4,5-b]pyridin-3-yl]piperidine-2-carboxylate

Starting from Preparation 2a following General procedure II and using ethyl 4-aminopiperidine-1-carboxylate as the appropriate amine Example 35 was obtained. HRMS (TOF, ESI) m/z: Calculated for C₂₀H₂₅N₇O₂ 395.2070, Found: 396.2152 [M+H]⁺.

### Example 36 4-[2-methyl-3-(5,6,7,8-tetrahydroquinolin-5-yl)-3H-imidazo[4,5-b]pyridin-5-yl]pyridine-2,6-diamine

Starting from Preparation 2a following General procedure II and using 5,6,7,8-tetrahydroquinolin-5-amine as the appropriate amine Example 36 was obtained. HRMS (IT-TOF, ESI) m/2: Calculated for C₂₁H₂₁N₇ 371.1858, Found: 372.1939 [M+H]⁺.

### Example 37 3-[5-(2,6-diaminopyridin-4-yl)-2-methyl-3H-imidazo[4,5-b]pyridin-3-yl]propane-1,2-diol

Starting from Preparation 2a following General procedure II and using 3-aminopropane-1,2-diol as the appropriate amine Example 37 was obtained. HRMS (IT-TOF, ESI) m/z: Calculated for C₁₅H₁₈N₆O₂ 314.1491, Found: 315.1559 [M+H]⁺.

### Example 38 1-{4-[5-(2,6-diaminopyridin-4-yl)-2-methyl-3H-imidazo[4,5-b]pyridin-3-yl]piperidin-1-yl}-2-methylpropan-1-one

Starting from Preparation 2a following General procedure II and using 1-(4-amino-1-piperidyl)-2-methyl-propan-1-one as the appropriate amine Example 38 was obtained. HRMS (IT-TOF, ESI) m/z: Calculated for C₂₁H₂₇N₇O 393.2277, Found: 394.2356 [M+H]⁺.

### Example 39 4-[3-(4-chloro-2-methoxybenzyl)-2-methyl-3H-imidazo[4,5-b]pyridin-5-yl]pyridine-2,6-diamine

Starting from Preparation 2a following General procedure II and using (4-chloro-2-methoxy-phenyl)methanamine as the appropriate amine Example 39 was obtained. HRMS (IT-TOF, ESI) m/z: Calculated for C₂₀H₁₉N₆OCl 394.1309, Found: 395.1387 [M+H]⁺.

### Example 40 4-{[5-(2,6-diaminopyridin-4-yl)-2-methyl-3H-imidazo[4,5-b]pyridin-3-yl]methyl}benzonitrile

Starting from Preparation 2a following General procedure II and using 4-(aminomethyl)benzonitrile as the appropriate amine Example 40 was obtained. HRMS (IT-TOF, ESI) m/z: Calculated for C₂₀H₁₇N₇ 355.1545, Found: 356.1612 [M+H]⁺.

### Example 41 4-[2-methyl-3-(tetrahydrofuran-3-ylmethyl)-3H-imidazo[4,5-b]pyridin-5-yl]pyridine-2,6-diamine

Starting from Preparation 2a following General procedure II and using tetrahydrofuran-3-ylmethanamine as the appropriate amine Example 41 was obtained. HRMS (IT-TOF, ESI) m/z: Calculated for C₁₇H₂₀N₆O 324.1699, Found: 325.1787 [M+H]⁺.

### Example 42 4-[3-(furan-3-ylmethyl)-2-methyl-3H-imidazo[4,5-b]pyridin-5-yl]pyridine-2,6-diamine

Starting from Preparation 2a following General procedure 11 and using 3-furylmethanamine as the appropriate amine Example 42 was obtained. HRMS (IT-TOF, ESI) m/z: Calculated for C₁₇H₁₆N₆O 320.1386, Found: 321.1467 [M+H]⁺.

### Example 43 4-{3-[4-(difluoromethoxy)benzyl]-2-methyl-3H-imidazo[4,5-b]pyridin-5-yl}pyridine-2,6-diamine

Starting from Preparation 2a following General procedure II and using [4-(difluoromethoxy)phenyl]methanamine as the appropriate amine Example 43 was obtained. HRMS (IT-TOF, ESI) m/z: Calculated for C₂₀H₁₈N₆OF₂ 396.1510, Found: 397.1581 [M+H]⁺.

### Example 44 4-{2-methyl-3-[3-(methylsulfanyl)propyl]-3H-imidazo[4,5-b]pyridin-5-yl}pyridine-2,6-diamine

Starting from Preparation 2a following General procedure II and using 3-methylsulfanylpropan-1-amine as the appropriate amine Example 44 was obtained. HRMS (IT-TOF, ESI) m/z: Calculated for C₁₆H₂₀N₆S 328.1470, Found: 329.1551 [M+H]⁺.

### Example 45 4-{2-methyl-3-[(3,5-trimethyl-1H-pyrazol-4-yl)methyl]-3H-imidazo[4,5-b]pyridin-5-yl}pyridine-2,6-diamine

Starting from Preparation 2a following General procedure 11 and using (1,3,5-trimethyl-1H-pyrazol-4-yl)methanamine as the appropriate amine Example 45 was obtained. HRMS (IT-TOF, ESI) m/z: Calculated for C₁₉H₂₂N₈ 362.1967, Found: 363.2037 [M+H]⁺.

### Example 46 4-[3-(2,5-difluorobenzyl)-2-methyl-3H-imidazo[4,5-b]pyridin-5-yl]pyridine-2,6-diamine

Starting from Preparation 2a following General procedure II and using (2,5-difluorophenyl)methanamine as the appropriate amine Example 46 was obtained. HRMS (IT-TOF, ESI) m/z: Calculated for C₁₉H₁₆N₆F₂ 366.1405, Found: 367.1483 [M+H]⁺.

### Example 47 4-[3-(2-chlorobenzyl)-2-methyl-3H-imidazo[4,5-b]pyridin-5-y]pyridine-2,6-diamine

Starting from Preparation 2a following General procedure II and using (2-chlorophenyl)methanamine as the appropriate amine Example 47 was obtained. HRMS (IT-TOF, ESI) m/z: Calculated for C₁₉H₁₇N₆Cl 364.1203, Found: 365.1279 [M+H]⁺.

### Example 48 4-[3-(3-chlorobenzyl)-2-methyl-3H-imidazo[4,5-b]pyridin-5-yl]pyridine-2,6-diamine

Starting from Preparation 2a following General procedure I and using (3-chlorophenyl)methanamine as the appropriate amine Example 48 was obtained. HRMS (IT-TOF, ESI) m/z: Calculated for C₁₉H₁₇N₆Cl 364.1203, Found: 365.1277 [M+H]⁺.

### Example 49 4-[2-methyl-3-(tetrahydro-2H-pyran-4-yl)-3H-imidazo[4,5-b]pyridin-5-yl]pyridine-2,6-diamine

Starting from Preparation 2a following General procedure II and using tetrahydro-2H-pyran-4-amine as the appropriate amine Example 49 was obtained. HRMS (TOF, ESI) m/z: Calculated for C₁₇H₂₀N₆O 324.12699, Found: 325.1790 [M+H]⁺.

### Example 50 4-{3-[2-fluoro-5-(trifluoromethoxy)benzyl]-2-methyl-3H-imidazo[4,5-b]pyridin-5-yl}pyridine-2,6-diamine

Starting from Preparation 2a following General procedure 11 and using [2-fluoro-5-(trifluoromethoxy)phenyl]methanamine as the appropriate amine Example 50 was obtained. HRMS (TOF, ESI) m/z: Calculated for C₂₀H₁₆N₆OF₄ 432.1322, Found: 433.1416 [M+H]⁺.

### Example 51 4-[2-methyl-3-(prop-2-en-1-yl)-3H-imidazo[4,5-b]pyridin-5-yl]pyridine-2,6-diamine

Starting from Preparation 2a following General procedure II and using prop-2-en-1-amine as the appropriate amine Example 51 was obtained. HRMS (TOF, ESI) m/z: Calculated for C₁₅H₁₆N₆ 280.1436, Found: 281.1524 [M+H]⁺.

### Example 52 4-[3-(3,3-dimethylbutyl)-2-methyl-3H-imidazo[4,5-b]pyridin-5-yl]pyridine-2,6-diamine

Starting from Preparation 2a following General procedure II and using 3,3-dimethylbutan-1-amine as the appropriate amine Example 52 was obtained. HRMS (TOF, ESI) m/z: Calculated for C₁₈H₂₄N₆ 324.2062, Found: 325.2153 [M+H]⁺.

### Example 53 4-[2-methyl-3-(propan-2-yl)-3H-imidazo[4,5-b]pyridin-5-yl]pyridine-2,6-diamine

Starting from Preparation 2a following General procedure II and using propan-2-amine as the appropriate amine Example 53 was obtained. HRMS (TOF, ESI) m/z: Calculated for C₁₅H₁₈N₆ 282.1593, Found: 283.1675 [M+H]⁺.

### Example 54 4-(3-cyclohexyl-2-methyl-3H-imidazo[4,5-b]pyridin-5-yl)pyridine-2,6-diamine

Starting from Preparation 2a following General procedure II and using cyclohexanamine as the appropriate amine Example 54 was obtained. HRMS (TOF, ESI) m/z: Calculated for C₁₈H₂₂N₆ 323.1921, Found: 323.1994 [M+H]⁺.

### Example 55 4-[3-(cyclohexylmethyl)-2-methyl-3H-imidazo[4,5-b]pyridin-5-yl]pyridine-2,6-diamine

Starting from Preparation 2a following General procedure II and using 1-cyclohexylmethanamine as the appropriate amine Example 55 was obtained. HRMS (TOF, ESI) m/z: Calculated for C₁₉H₂₄N₆ 336.2062, Found: 337.2151 [M+H]⁺.

### Example 56 4-{2-methyl-3-tricyclo[3.3.1.1^{3,7}]dec-1-yl-3H-imidazo[4,5-b]pyridin-5-yl}pyridine-2,6-diamine

Starting from Preparation 2a following General procedure II and using 1-adamantylamine as the appropriate amine Example 56 was obtained. HRMS (TOF, ESI) m/z: Calculated for C₂₂H₂₆N₆ [M+H]⁺ 374.2219, Found: 375.2307.

### Example 57 4-[3-(2,5-dichlorobenzyl)-2-methyl-3H-imidazo[4,5-b]pyridin-5-yl]pyridine-2,6-diamine

Starting from Preparation 2a following General procedure II and using (2,5-dichlorophenyl)methanamine as the appropriate amine Example 57 was obtained. HRMS (TOF, ESI) m/z: Calculated for C₁₉H₁₆N₆Cl₂ 398.0814, Found: 399.0896 [M+H]⁺.

### Example 58 4-{3-[2-(3,4-dichlorophenyl)ethyl]-2-methyl-3H-imidazo[4,5-b]pyridin-5-yl}pyridine-2,6-diamine

Starting from Preparation 2a following General procedure 11 and using 2-(3,4-dichlorophenyl)ethanamine as the appropriate amine Example 58 was obtained. HRMS (TOF, ESI) m/z: Calculated for C₂₀H₁₈N₆Cl₂ 412.0970, Found: 413.1053 [M+H]⁺.

### Example 59 4-{3-[2-(2,4-dichlorophenyl)ethyl]-2-methyl-3H-imidazo[4,5-b]pyridin-5-yl}pyridine-2,6-diamine

Starting from Preparation 2a following General procedure II and using 2-(2,4-dichlorophenyl)ethanamine as the appropriate amine Example 59 was obtained. HRMS (TOF, ESI) m/z: Calculated for C₂₀H₁₈N₆Cl₂ 412.097, Found: 413.1056 [M+H]⁺.

### Example 60 4-[2-methyl-3-(2-phenylpropan-2-yl)-3H-imidazo[4,5-b]pyridin-5-yl]pyridine-2,6-diamine

and

### Example 61 4-(2-methyl-3H-imidazo[4,5-b]pyridin-5-yl)pyridine-2,6-diamine

Starting from Preparation 2a following General procedure II and using 2-phenylpropan-2-amine as the appropriate amine Example 60 was obtained. HRMS (TOF, ESI) m/z: Calculated for C₂₁H₂₂N₆ 358.1906, Found: 359.1988 [M+H]⁺ From the same reaction Example 61 was also isolated. HRMS (TOF, ESI) m/z: Calculated for C₁₂H₁₂N₆ 240.1123, Found: 241.1206. [M+H]⁺.

### Example 62 4-[2-methyl-3-(1,2,3,4-tetrahydronaphthalen-1-yl)-3H-imidazo[4,5-b]pyridin-5-yl]pyridine-2,6-diamine

Starting from Preparation 2a following General procedure II and using 1,2,3,4-tetrahydronaphthalen-1-amine as the appropriate amine Example 62 was obtained. HRMS (TOF, ESI) m/z: Calculated for C₂₂H₂₂N₆ 370.1906, Found: 371.1989 [M+H]⁺.

### Example 63 4-{2-methyl-3-[2-(2-methylphenyl)ethyl]-3H-imidazo[4,5-b]pyridin-5-yl}pyridine-2,6-diamine

Starting from Preparation 2a following General procedure 11 and using 2-(o-tolyl)ethanamine as the appropriate amine Example 63 was obtained. HRMS (TOF, ESI) m/z: Calculated for C₂₁H₂₂N₆ 358.1906, Found: 359.1985 [M+H]⁺.

### Example 64 4-[2-methyl-3-(pentan-2-yl)-3H-imidazo[4,5-b]pyridin-5-yl]pyridine-2,6-diamine

Starting from Preparation 2a following General procedure II and using pentan-2-amine as the appropriate amine Example 64 was obtained. HRMS (TOF, ESI) m/z: Calculated for C₁₇H₂₂N₆ 310.1906, Found: 311.1980 [M+H]⁺.

### Example 65 4-(2-methyl-3-pentyl-3H-imidazo[4,5-b]pyridin-5-yl)pyridine-2,6-diamine

Starting from Preparation 2a following General procedure II and using pentan-1-amine as the appropriate amine Example 65 was obtained. HRMS (TOF, ESI) m/z: Calculated for C₁₇H₂₂N₆ 310.1906, Found: 311.1983 [M+H]⁺.

### Example 66 4-[2-methyl-3-(tetrahydro-2H-thiopyran-4-yl)-3H-imidazo[4,5-b]pyridin-5-yl]pyridine-2,6-diamine

Starting from Preparation 2a following General procedure II and using tetrahydro-2H-thiopyran-4-amine as the appropriate amine Example 66 was obtained. HRMS (TOF, ESI) m/z: Calculated for C₁₇H₂₀N₆S 340.147, Found: 341.1545 [M+H]⁺.

### Example 67 4-[2-methyl-3-(1-phenylpropyl)-3H-imidazo[4,5-b]pyridin-5-yl]pyridine-2,6-diamine

Starting from Preparation 2a following General procedure II and using 1-phenylpropan-1-amine as the appropriate amine Example 67 was obtained. HRMS (TOF, ESI) m/z: Calculated for C₂₁1H₂₂N₆ 358.1906, Found: 359.1979 [M+H]⁺.

### Example 68 4-[2-methyl-3-(pentan-3-yl)-3H-imidazo[4,5-b]pyridin-5-yl]pyridine-2,6-diamine

Starting from Preparation 2a following General procedure II and using pentan-3-amine as the appropriate amine Example 68 was obtained. HRMS (TOF, ESI) m/z: Calculated for C₁₇H₂₂N₆ 310.1906, Found: 311.1985 [M+H]⁺.

### Example 69 4-{3-[3-(2-methoxyphenyl)propyl]-2-methyl-3H-imidazo[4,5-b]pyridin-5-yl}pyridine-2,6-diamine

Starting from Preparation 2a following General procedure II and using 3-(2-methoxyphenyl)propan-1-amine as the appropriate amine Example 69 was obtained. HRMS (TOF, ESI) m/z: Calculated for C₂₂H₂₄N₆O 388.2012, Found: 389.2095 [M+H]⁺.

### Example 70 4-[5-(2,6-diaminopyridin-4-yl)-2-methyl-3H-imidazo[4,5-b]pyridin-3-yl]butan-1-ol

Starting from Preparation 2a following General procedure II and using 4-aminobutan-1-ol as the appropriate amine Example 70 was obtained. HRMS (TOF, ESI) m/z: Calculated for C₁₆H₂₀N₆O 312.1699, Found: 313.1767 [M+H]⁺.

### Example 71 4-[2-methyl-3-(4,4,4-trifluorobutyl)-3H-imidazo[4,5-b]pyridin-5-yl]pyridine-2,6-diamine

Starting from Preparation 2a following General procedure II and using 4,4,4-trifluorobutan-1-amine as the appropriate amine Example 71 was obtained. HRMS (TOF, ESI) m/z: Calculated for C₁₆H₁₇N₆F₃ 350.1467, Found: 351.1533 [M+H]⁺.

### Example 72 4-{3-[(2-methoxypyridin-4-yl)methyl]-2-methyl-3H-imidazo[4,5-b]pyridin-5-yl}pyridine-2,6-diamine

Starting from Preparation 2a following General procedure II and using (2-methoxy-4-pyridyl)methanamine as the appropriate amine Example 72 was obtained. HRMS (TOF, ESI) m/z: Calculated for C₁₉H₁₉N₇O 361.1651, Found: 362.1726 [M+H]⁺.

### Example 73 4-(3-[2-(1,3-benzodioxol-5-yl)ethyl]-2-methyl-3H-imidazo[4,5-b]pyridin-5-yl)pyridine-2,6-diamine

Starting from Preparation 2a following General procedure II and using 2-(1,3-benzodioxol-5-yl)ethanamine as the appropriate amine Example 73 was obtained. HRMS (TOF, ESI) m/z: Calculated for C₂₁H₂₀N₆O₂ 388.1648, Found: 389.1728 [M+H]⁺.

### Example 74 4-{3-[(2,2-dichlorocyclopropyl)methyl]-2-methyl-3H-imidazo[4,5-b]pyridin-5-yl}pyridine-2,6-diamine

Starting from Preparation 2a following General procedure II and using (2,2-dichlorocyclopropyl)methanamine as the appropriate amine Example 74 was obtained. HRMS (TOF, ESI) m/z: Calculated for C₁₆H₁₆N₆Cl₂ 362.0814, Found: 363.0883 [M+H]⁺.

### Example 75 4-[2-methyl-3-(3-methylbutyl)-3H-imidazo[4,5-b]pyridin-5-yl]pyridine-2,6-diamine

Starting from Preparation 2a following General procedure II and using 3-methylbutan-1-amine as the appropriate amine Example 75 was obtained. HRMS (TOF, ESI) m/z: Calculated for C₁H₂₂N₆ 310.1906, Found: 311.1990 [M+H]⁺.

### Example 76 4-[2-methyl-3-(tetrahydro-2H-pyran-3-ylmethyl)-3H-imidazo[4,5-b]pyridin-5-yl]pyridine-2,6-diamine

Starting from Preparation 2a following General procedure II and using tetrahydro-2H-pyran-3-ylmethanamine as the appropriate amine Example 76 was obtained. HRMS (TOF, ESI) m/z: Calculated for C₁₈H₂₂N₆O 338.1855, Found: 339.1941 [M+H]⁺.

### Example 77 4-{3-[2-(2,3-dihydro-1,4-benzodioxin-6-yl)ethyl]-2-methyl-3H-imidazo[4,5-b]pyridin-5-yl}pyridine-2,6-diamine

Starting from Preparation 2a following General procedure II and using 2-(2,3-dihydro-1,4-benzodioxin-6-yl)ethanamine as the appropriate amine Example 77 was obtained. HRMS (TOF, ESI) m/z: Calculated for C₂₂H₂₂N₆O₂ 402.1804, Found: 403.1888 [M+H]⁺.

### Example 78 4-{2-methyl-3-[2-(tetrahydro-2H-pyran-4-yl)ethyl]-3H-imidazo[4,5-b]pyridin-5-yl}pyridine-2,6-diamine

Starting from Preparation 2a following General procedure II and using 2-tetrahydro-2H-pyran-4-ylethanamine as the appropriate amine Example 78 was obtained. HRMS (TOF, ESI) m/z: Calculated for C₁₉H₂₄N₆O 352.2012, Found: 353.2080 [M+H]⁺.

### Example 79 4-(2-methyl-3-{2-[2-(trifluoromethyl)phenyl]ethyl}-3H-imidazo[4,5-b]pyridin-5-yl)pyridine-2,6-diamine

Starting from Preparation 2a following General procedure II and using 2-[2-(trifluoromethyl)phenyl]ethanamine as the appropriate amine Example 79 was obtained. HRMS (TOF, ESI) m/z: Calculated for C₂₁H₁₉N₆F₃ 412.1623, Found: 413.1708 [M+H]⁺.

### Example 80 4-[2-methyl-3-(tetrahydro-2H-pyran-4-ylmethyl)-3H-imidazo[4,5-b]pyridin-5-yl]pyridine-2,6-diamine

Starting from Preparation 2a following General procedure II and using tetrahydro-2H-pyran-4-ylmethanamine as the appropriate amine Example 80 was obtained. HRMS (TOF, ESI) m/z: Calculated for C₁₈H₂₂N₆O 338.1855, Found: 339.1929 [M+H]⁺.

### Example 81 4-{3-[2-(2-methoxyphenyl)ethyl]-2-methyl-3H-imidazo[4,5-b]pyridin-5-yl}pyridine-2,6-diamine

Starting from Preparation 2a following General procedure II and using 2-(2-methoxyphenyl)ethanamine as the appropriate amine Example 81 was obtained. HRMS (TOF, ESI) m/z: Calculated for C₂₁H₂₂N₆O 374.1855, Found: 375.1924 [M+H]⁺.

### Example 82 4-{3-[1-(furan-2-yl)propan-2-yl]-2-methyl-3H-imidazo[4,5-b]pyridin-5-yl}pyridine-2,6-diamine

Starting from Preparation 2a following General procedure II and using 1-(2-furyl)propan-2-amine as the appropriate amine Example 82 was obtained. HRMS (TOF, ESI) m/z: Calculated for C₁₉H₂₀N₆O 348.1699, Found: 349.1772 [M+H]⁺.

### Example 83 4-[2-methyl-3-(2-phenylethyl)-3H-imidazo[4,5-b]pyridin-5-yl]pyridine-2,6-diamine

Starting from Preparation 2a following General procedure II and using 2-phenyl-ethylamine as the appropriate amine Example 83 was obtained. HRMS (TOF, ESI) m/z: Calculated for C₂₀H₂₀N₆ 344.1749, Found: 345.1829 [M+H]⁺.

### Example 84 4-{3-[(2-fluoropyridin-4-yl)methyl]-2-methyl-3H-imidazo[4,5-b]pyridin-5-yl}pyridine-2,6-diamine

Starting from Preparation 2a following General procedure II and using (2-fluoro-4-pyridyl)methanamine as the appropriate amine Example 84 was obtained. HRMS (TOF, ESI) m/z: Calculated for C₁₈H₁₆N₇F 349.1451, Found: 350.1531 [M+H]⁺.

### Example 85 4-{2-methyl-3-[2-(tetrahydrofuran-2-yl)ethyl]-3H-imidazo[4,5-b]pyridin-5-yl}pyridine-2,6-diamine

Starting from Preparation 2a following General procedure II and using 2-tetrahydrofuran-2-ylethanamine as the appropriate amine Example 85 was obtained. HRMS (TOF, ESI) m/z: Calculated for C₁₈H₂₂N₆O 338.1855, Found: 339.1930 [M+H]⁺.

### Example 86 4-{2-methyl-3-[(2-methylcyclopropyl)methyl]-3H-imidazo[4,5-b]pyridin-5-yl}pyridine-2,6-diamine

Starting from Preparation 2a following General procedure II and using 1-(2-methylcyclopropyl)methanamine as the appropriate amine Example 86 was obtained. HRMS (TOF, ESI) m/z: Calculated for C₁₇H₂₀N₆ 308.1749, Found: 309.1822 [M+H]⁺.

### Example 87 4-(2-methyl-3-(2-[3-(propan-2-yloxy)phenyl]ethyl-3H-imidazo[4,5-b]pyridin-5-yl)pyridine-2,6-diamine

Starting from Preparation 2a following General procedure II and using 2-(3-isopropoxyphenyl)ethanamine as the appropriate amine Example 87 was obtained. HRMS (TOF, ESI) m/z: Calculated for C₂₃H₂₆N₆O 402.2168, Found: 403.2153 [M+H]⁺.

### Example 88 4-{3-[(1-ethylcyclopropyl)methyl]-2-methyl-3H-imidazo[4,5-b]pyridin-5-yl}pyridine-2,6-diamine

Starting from Preparation 2a following General procedure I and using 1-(1-ethylcyclopropyl)methanamine as the appropriate amine Example 88 was obtained. HRMS (TOF, ESI) m/z: Calculated for C₁₈H₂₂N₆ 322.1906, Found: 323.1988 [M+H]⁺.

### Example 89 4-[3-(cyclopentylmethyl)-2-methyl-3H-imidazo[4,5-b]pyridin-5-yl]pyridine-2,6-diamine

Starting from Preparation 2a following General procedure II and using cyclopentylmethanamine as the appropriate amine Example 89 was obtained. HRMS (TOF, ESI) m/z: Calculated for C₁₈H₂₂N₆ 322.1906, Found: 323.1987 [M+H]⁺.

### Example 90 4-{3-[2-(3-ethoxyphenyl)ethyl]-2-methyl-3H-imidazo[4,5-b]pyridin-5-yl}pyridine-2,6-diamine

Starting from Preparation 2a following General procedure II and using 2-(3-ethoxyphenyl)ethanamine as the appropriate amine Example 90 was obtained. HRMS (TOF, ESI) m/z: Calculated for C₂₂H₂₄N₆O 388.2012, Found: 389.1953 [M+H]⁺.

### Example 91 4-(2-methyl-3-{2-[3-(trifluoromethyl)phenyl]ethyl}-3H-imidazo[4,5-b]pyridin-5-yl)pyridine-2,6-diamine

Starting from Preparation 2a following General procedure II and using 2-[3-(trifluoromethyl)phenyl]ethanamine as the appropriate amine Example 91 was obtained. HRMS (TOF, ESI) m/z: Calculated for C₂₁H₁₉N₆F₃ 412.1623, Found: 413.1683 [M+H]⁺.

### Example 92 4-[3-(2-cyclopentylethyl)-2-methyl-3H-imidazo[4,5-b]pyridin-5-yl]pyridine-2,6-diamine

Starting from Preparation 2a following General procedure II and using 2-cyclopentylethanamine as the appropriate amine Example 92 was obtained. HRMS (TOF, ESI) m/z: Calculated for C₁₉H₂₄N₆ 336.2062, Found: 337.2012 [M+H]⁺.

### Example 93 4-[3-(5-methoxy-1,2,3,4-tetrahydronaphthalen-2-yl)-2-methyl-3H-imidazo[4,5-b]pyridin-5-yl]pyridine-2,6-diamine

Starting from Preparation 2a following General procedure II and using 5-methoxytetralin-2-amine as the appropriate amine Example 93 was obtained. HRMS (TOF, ESI) m/z: Calculated for C₂₃H₂₄N₆O 400.2012, Found: 401.1963 [M+H]⁺.

### Example 94 4-(3-hexyl-2-methyl-3H-imidazo[4,5-b]pyridin-5-yl)pyridine-2,6-diamine

Starting from Preparation 2a following General procedure II and using hexan-1-amine as the appropriate amine Example 94 was obtained. HRMS (TOF, ESI) m/z: Calculated for C₁₈H₂₄N₆ 324.2062, Found: 325.2014 [M+H]⁺.

### Example 95 4-{3-[2-(2-methoxycyclohexyl)ethyl]-2-methyl-3H-imidazo[4,5-b]pyridin-5-yl}pyridine-2,6-diamine

Starting from Preparation 2a following General procedure 11 and using 2-(2-methoxycyclohexyl)ethanamine as the appropriate amine Example 95 was obtained. HRMS (TOF, ESI) m/z: Calculated for C₂₁H₂₈N₆O 380.2325, Found: 381.2378 [M+H]⁺.

### Example 96 4-{3-[2-(4-fluorophenyl)ethyl]-2-methyl-3H-imidazo[4,5-b]pyridin-5-yl}pyridine-2,6-diamine

Starting from Preparation 2a following General procedure II and using 2-(4-fluorophenyl)ethanamine as the appropriate amine Example 96 was obtained. HRMS (TOF, ESI) m/z: Calculated for C₂₀H₁₉N₆F 362.1655, Found: 363.1726 [M+H]⁺.

### Example 97 4-{2-methyl-3-[(2-phenylcyclopropyl)methyl]-3H-imidazo[4,5-b]pyridin-5-yl}pyridine-2,6-diamine

Starting from Preparation 2a following General procedure II and using 1-(2-phenylcyclopropyl)methanamine as the appropriate amine Example 97 was obtained. HRMS (TOF, ESI) m/z: Calculated for C₂₂H₂₂N₆ 370.1906, Found: 371.1976 [M+H]⁺.

### Example 98 4-[3-(5-methoxy-2,3-dihydro-1H-inden-2-yl)-2-methyl-3H-imidazo[4,5-b]pyridin-5-yl]pyridine-2,6-diamine

Starting from Preparation 2a following General procedure II and using 5-methoxyindan-2-amine as the appropriate amine Example 98 was obtained. HRMS (TOF, ESI) m/z: Calculated for C₂₂H₂₂N₆O 386.1855, Found: 387.1818 [M+H]⁺.

### Example 99 4-{3-[(2,2-dimethylcyclopropyl)methyl]-2-methyl-3H-imidazo[4,5-b]pyridin-5-yl}pyridine-2,6-diamine

Starting from Preparation 2a following General procedure II and using 1-(2,2-dimethylcyclopropyl)methanamine as the appropriate amine Example 99 was obtained. HRMS (TOF, ESI) m/z: Calculated for C₁₈H₂₂N₆ 322.1906, Found: 323.1978 [M+H]⁺.

### Example 100 4-[2-methyl-3-(3-phenylcyclobutyl)-3H-imidazo[4,5-b]pyridin-5-yl]pyridine-2,6-diamine

Starting from Preparation 2a following General procedure 11 and using 3-phenylcyclobutanamine as the appropriate amine Example 100 was obtained. HRMS (TOF, ESI) m/z: Calculated for C₂₂H₂₂N₆ 370.1906, Found: 371.1978 [M+H]⁺.

### Example 101 4-[3-(3,3-difluorocyclobutyl)-2-methyl-3H-imidazo[4,5-b]pyridin-5-yl]pyridine-2,6-diamine

Starting from Preparation 2a following General procedure II and using 3,3-difluorocyclobutanamine as the appropriate amine Example 101 was obtained. HRMS (TOF, ESI) m/z: Calculated for C₁₆H₁₆N₆F₂ 330.1405, Found: 331.1463 [M+H]⁺.

### Example 102 4-{2-methyl-3-[2-(2-methylcyclohexyl)ethyl]-3H-imidazo[4,5-b]pyridin-5-yl}pyridine-2,6-diamine

Starting from Preparation 2a following General procedure II and using 2-(2-methylcyclohexyl)ethanamine as the appropriate amine Example 102 was obtained. HRMS (TOF, ESI) m/z: Calculated for C₂₁H₂₈N₆ 364.2375, Found: 365.2447 and 365.2436 [M+H]⁺ for the two diastereoisomers.

### Example 103 4-[3-(3-fluorocyclobutyl)-2-methyl-3H-imidazo[4,5-b]pyridin-5-yl]pyridine-2,6-diamine

Starting from Preparation 2a following General procedure II and using 3-fluorocyclobutanamine as the appropriate amine Example 103 was obtained. HRMS (TOF, ESI) m/z: Calculated for C₁₆H₁₇N₆F 312.1499, Found: 313.1566 [M+H]⁺.

### Example 104 4-{2-methyl-3-[(2R)-1-phenoxypropan-2-yl]-3H-imidazo[4,5-b]pyridin-5-yl}pyridine-2,6-diamine

and

### Example 105 4-{2-methyl-3-[(2S)-1-phenoxypropan-2-yl]-3H-imidazo[4,5-b]pyridin-5-yl}pyridine-2,6-diamine

Starting from Preparation 2a following General procedure II and using 1-phenoxypropan-2-amine as the appropriate amine a mixture of Example 104 and Example 105 was obtained. The enantiomers were separated on CHIRALCEL OK column using MeOH+0.1% DEA as eluent to obtain Example 104 as the first eluting enantiomer. HRMS (TOF, ESI) m/z: Calculated for C₂₁H₂₂N₆O 374.1855, Found: 375.1913 [M+H]⁺ ee=99.8% (E1). Example 105 was obtained as the second eluting enantiomer. HRMS (TOF, ESI) m/z: Calculated for C₂₁H₂₂N₆O 374.1844, Found: 375.1917 [M+H]⁺ ee=98.4% (E2).

### Example 106 4-{2-methyl-3-[(2R)-2-phenoxypropyl]-3H-imidazo[4,5-b]pyridin-5-yl}pyridine-2,6-diamine

and

### Example 107 4-{2-methyl-3-[(2S)-2-phenoxypropyl]-3H-imidazo[4,5-b]pyridin-5-yl}pyridine-2,6-diamine

Starting from Preparation 2a following General procedure II and using 2-phenoxypropan-1-amine as the appropriate amine a mixture of Example 106 and Example 107 was obtained. The enantiomers were separated on CHIRALCEL OK column using MeOH+0.1% DEA as eluent to obtain Example 106 as the first eluting enantiomer. HRMS (TOF, ESI) m/z: Calculated for C₂₁H₂₂N₆O 374.1855, Found: 375.1924. [M+H]⁺ ee=99.8% (E1). Example 107 was obtained as the second eluting enantiomer. HRMS (TOF, ESI) m/z: Calculated for C₂₁H₂₂N₆O 374.1855, Found: 375.1922 [M+H]⁺ ee=99.8% (E2).

### Example 108 4-[2-methyl-3-(3,3,3-trifluoropropyl)-3H-imidazo[4,5-b]pyridin-5-yl]pyridine-2,6-diamine

Starting from Preparation 2a following General procedure II and using 3,3,3-trifluoropropan-1-amine as the appropriate amine Example 108 was obtained. HRMS (IT-TOF, ESI) m/z: Calculated for C₁₅H₁₅N₆F₃ 336.131, Found: 337.1381 [M+H]⁺.

### Example 109 4-(2-methyl-3-{[(1 S,2S)-2-phenylcyclopropyl]methyl}-3H-imidazo[4,5-b]pyridin-5-yl)pyridine-2,6-diamine

and

### Example 110 4-(2-methyl-3-{[(1R,2R)-2-phenylcyclopropyl]methyl}-3H-imidazo[4,5-b]pyridin-5-yl)pyridine-2,6-diamine

Starting from Preparation 2a following General procedure II and using [1,2-trans-2-phenylcyclopropyl]methanamine as the appropriate amine a mixture of Example 109 and Example 110 was obtained. The enantiomers were separated on CHIRALCEL OD-H column using 40:60 l-PrOH/heptane+0.1% DEA as eluent to obtain Example 109 as the first eluting enantiomer. HRMS (IT-TOF, ESI) m/z: Calculated for C₂₂H₂₂N₆ 370.1906, Found: 371.1981 [M+H]⁺ ee=99.8% (E1). Example 110 was obtained as the second eluting enantiomer. HRMS (IT-TOF, ESI) m/z: Calculated for C₂₂H₂₂N₆ 370.1906, Found: 371.1984 [M+H]⁺ ee=99.8% (E2).

### Example 111 4-{2-methyl-3-[(2E)-3-phenylprop-2-en-1-yl]-3H-imidazo[4,5-b]pyridin-5-yl}pyridine-2,6-diamine

Starting from Preparation 2a following General procedure 11 and using (E)-3-phenylprop-2-en-1-amine as the appropriate amine Example 111 was obtained. HRMS (IT-TOF, ESI) m/z: Calculated for C₂₁H₂₀N₆ 356.1749, Found: 357.1828 [M+H]⁺.

### Example 112 4-[3-(bicyclo[4.2.0]octa-1,3,5-trien-7-ylmethyl)-2-methyl-3H-imidazo[4,5-b]pyridin-5-yl]pyridine-2,6-diamine

and

### Example 113 4-[3-(bicyclo[4.2.0]octa-1,3,5-trien-7-ylmethyl)-2-methyl-3H-imidazo[4,5-b]pyridin-5-yl]pyridine-2,6-diamine

Starting from Preparation 2a following General procedure II and using 1-(bicyclo[4.2.0]octa-1,3,5-trien-7-ylmethanamine as the appropriate amine a mixture of Example 112 and Example 113 was obtained. The enantiomers were separated on CHIRALPAK AS-H column using 50:50 EtOH/heptane+0.1% DEA as eluent to obtain Example 112 as the first eluting enantiomer. HRMS (IT-TOF, ESI) m/z: Calculated for C₂₁H₂₀N₆ 356.1749, Found: 357.1818 [M+H]⁺ ee =99.8% (E1). Example 113 was obtained as the second eluting enantiomer. HRMS (IT-TOF, ESI) m/z: Calculated for C₂₁H₂₀N₆ 356.1749, Found: 357.1810 [M+H]⁺ ee 99.6% (E2).

### Example 114 4-[3-(2,3-dihydro-1H-inden-2-ylmethyl)-2-methyl-3H-imidazo[4,5-b]pyridin-5-yl]pyridine-2,6-diamine

Starting from Preparation 2a following General procedure II and using indan-2-ylmethanamine as the appropriate amine Example 114 was obtained. HRMS (IT-TOF, ESI) m/z: Calculated for C₂₂H₂₂N₆ 370.1906, Found: 371.1962 [M+H]⁺.

### Example 115 4-[3-(2,2-difluoroethyl)-2-methyl-3H-imidazo[4,5-b]pyridin-5-yl]pyridine-2,6-diamine

Starting from Preparation 2a following General procedure II and using 2,2-difluoroethanamine as the appropriate amine Example 115 was obtained. HRMS (TOF, ESI) m/z: Calculated for C₁₄H₁₄N₆F₂ 304.1248, Found: 305.1318 [M+H]⁺.

### Example 116 4-[2-methyl-3-(2-methyl-2-phenoxypropyl)-3H-imidazo[4,5-b]pyridin-5-yl]pyridine-2,6-diamine

Starting from Preparation 2a following General procedure II and using 2-methyl-2-phenoxy-propan-1-amine as the appropriate amine Example 116 was obtained. HRMS (IT-TOF, ESI) m/z: Calculated for C₂₂H₂₄N₆O 388.2012, Found: 389.2069 [M+H]⁺.

### Example 117 4-{3-[(2S)-2-(2-chlorophenoxy)propyl]-2-methyl-3H-imidazo[4,5-b]pyridin-5-yl}pyridine-2,6-diamine

and

### Example 118 4-{3-[(2R)-2-(2-chlorophenoxy)propyl]-2-methyl-3H-imidazo[4,5-b]pyridin-5-yl}pyridine-2,6-diamine

Starting from Preparation 2a following General procedure 11 and using 2-(2-chlorophenoxy)propan-1-amine as the appropriate amine a mixture of Example 0.117 and Example 118 was obtained. The enantiomers were separated on CHIRALPAK AS-H column using 50:50 EtOH/heptane+0.1% DEA as eluent to obtain Example 117 as the first eluting enantiomer. HRMS (IT-TOF, ESI) m/z: Calculated for C₂₁H₂₁N₆OCl 408.1465, Found: 409.1558 [M+H]⁺ ee=99.8% (E1). Example 118 was obtained as the second eluting enantiomer. HRMS (IT-TOF, ESI) m/z: Calculated for C₂₁H₂₁N₆OCl 408.1465, Found: 409.1538 [M+H]⁺ ee=99.8% (E2).

### Example 119 4-{2-methyl-3-[(2S)-2-phenoxybutyl]-3H-imidazo[4,5-b]pyridin-5-yl}pyridine-2,6-diamine

and

### Example 120 4-{2-methyl-3-[(2R)-2-phenoxybutyl]-3H-imidazo[4,5-b]pyridin-5-yl}pyridine-2,6-diamine

Starting from Preparation 2a following General procedure II and using 2-phenoxybutan-1-amine as the appropriate amine a mixture of Example 119 and Example 120 was obtained. The enantiomers were separated on CHIRALPAK AS-V column using 40:60 EtOH/heptane+0.05% DEA as eluent to obtain Example 119 as the first eluting enantiomer. HRMS (TOF, ESI) m/z: Calculated for C₂₂H₂₄N₆O 388.2012, Found: 389.2084 [M+H]⁺ ee=99.8% (E1). Example 120 was obtained as the second eluting enantiomer. HRMS (TOF, ESI) m/z: Calculated for C₂H₂₄N₆O 388.2012, Found: 389.2093 [M+H]⁺ ee=99.2% (E2).

### Example 121 4-(2-methyl-3-{(2R)-2-[methyl(phenyl)amino]propyl}-3H-imidazo[4,5-b]pyridin-5-yl)pyridine-2,6-diamine

and

### Example 122 4-(2-methyl-3-{(2S)-2-[methyl(phenyl)amino]propyl}-3H-imidazo[4,5-b]pyridin-5-yl)pyridine-2,6-diamine

Starting from Preparation 2a following General procedure II and using N²-methyl-N²-phenyl-propane-1,2-diamine as the appropriate amine a mixture of Example 121 and Example 122 was obtained. The enantiomers were separated on CHIRALPAK LA column using 20:80 EtOH/heptane+0.1% DEA as eluent to obtain Example 122 as the first eluting enantiomer. HRMS (IT-TOF, ESI) m/z: Calculated for C₂₂H₂₅N₇ 387.2171, Found: 388.2253 [M+H]⁺ ee=99.8% (E1). Example 121 was obtained as the second eluting enantiomer. HRMS (IT-TOF, ESI) m/z: Calculated for C₂₂H₂₅N₇ 387.2171, Found: 388.2232 [M+H]⁺ ee=99.8% (E2).

### Example 123 4-{3-[(2S)-2-(3-fluorophenoxy)propyl]-2-methyl-3H-imidazo[4,5-b]pyridin-5-yl}pyridine-2,6-diamine

and

### Example 124 4-{3-[(2R)-2-(3-fluorophenoxy)propyl]-2-methyl-3H-imidazo[4,5-b]pyridin-5-yl}pyridine-2,6-diamine

Starting from Preparation 2a following General procedure II and using 2-(3-fluorophenoxy)propan-1-amine as the appropriate amine a mixture of Example 123 and Example 124 was obtained. The enantiomers were separated on CHIRALCEL OJ-H column using EtOH+0.1% DEA as eluent to obtain Example 123 as the first eluting enantiomer. HRMS (IT-TOF, ESI) m/z: Calculated for C₂₁H₂₁N₆OF 392.1761, Found: 393.1850 [M+H]⁺ ee=99.8% (E1). Example 124 was obtained as the second eluting enantiomer. HRMS (IT-TOF, ESI) m/z: Calculated for C₂₁H₂₁N₆OF 392.1761, Found: 393.1828 [M+H]⁺ ee=99.8% (E2).

### Example 125 4-{3-[(2S)-2-(3-methoxyphenoxy)propyl]-2-methyl-3H-imidazo[4,5-b]pyridin-5-yl}pyridine-2,6-diamine

and

### Example 126 4-{3-[(2R)-2-(3-methoxyphenoxy)propyl]-2-methyl-3H-imidazo[4,5-b]pyridin-5-yl}pyridine-2,6-diamine

Starting from Preparation 2a following General procedure II and using 2-(3-methoxyphenoxy)propan-1-amine as the appropriate amine a mixture of Example 125 and Example 126 was obtained. The enantiomers were separated on CHIRALPAK AS-H column to obtain Example 125 as the first eluting enantiomer. HRMS (IT-TOF, ESI) m/z: Calculated for C₂₂H₂₄N₆O₂ 404.1961, Found: 405.2040 [M+H]⁺ ee=99.8% (E1). Example 126 was obtained as the second eluting enantiomer. HRMS (IT-TOF, ESI) m/z: Calculated for C₂₂H₂₄N₆O₂ 404.1961, Found: 405.2048 [M+H]⁺=99.6% (E2).

### Example 127 4-{2-methyl-3-[(2S)-2-(3-methylphenoxy)propyl]0.3H-imidazo[4,5-b]pyridin-5-yl}pyridine-2,6-diamine

and

### Example 128 4-{2-methyl-3-[(2R)-2-(3-methylphenoxy)propyl]-3H-imidazo[4,5-b]pyridin-5-yl}pyridine-2,6-diamine

Starting from Preparation 2a following General procedure II and using 2-(3-methylphenoxy)propan-1-amine as the appropriate amine a mixture of Example 127 and Example 128 was obtained. The enantiomers were separated on CHIRALPAK AS-V column using 50:50 EtOH/heptane+0.05% DEA as eluent to obtain Example 127 as the 34) first eluting enantiomer. HRMS (IT-TOF, ESI) m/z: Calculated for C₂₂H₂₄N₆O 388.2012, Found: 389.2088. [M+H]⁺ ee=99.8% (E1). Example 128 was obtained as the second eluting enantiomer. HRMS (IT-TOF, ESI) m/z: Calculated for C₂₂H₂₄NO₆O 388.2012, Found: 389.2079. [M+H]⁺ ee=99.2% (E2).

### Example 129 4-{3-[(2S)-2-(4-fluorophenoxy)propyl]-2-methyl-3H-imidazo[4,5-b]pyridin-5-yl}pyridine-2,6-diamine

and

### Example 130 4-{3-[(2R)-2-(4-fluorophenoxy)propyl]-2-methyl-3H-imidazo[4,5-b]pyridin-5-yl}pyridine-2,6-diamine

Starting from Preparation 2a following General procedure II and using 2-(4-fluorophenoxy)propan-1-amine as the appropriate amine a mixture of Example 129 and Example 130 was obtained. The enantiomers were separated on CHIRALPAK AS-V column using 50:50 EtOH/heptane+0.05% DEA as eluent to obtain Example 129 as the first eluting enantiomer. HRMS (IT-TOF, ESI) m/z: Calculated for C₂₁H₂₁N₆OF 392.1761, Found: 393.1832. [M+H]⁺ ee=99.8% (E1). Example 130 was obtained as the second eluting enantiomer. HRMS (IT-TOF, ESI) m/z: Calculated for C₂₁H₂₁N₆OF 392.1761, Found: 393.1834. [M+H]⁺ ee=99.8% (E2).

### Example 131 4-{2-methyl-3-[(2S)-2-(2-methylphenoxy)propyl]-3H-imidazo[4,5-b]pyridin-5-yl}pyridine-2,6-diamine

and

### Example 132 4-{2-methyl-3-[(2R)-2-(2-methylphenoxy)propyl]-3H-imidazo[4,5-b]pyridin-5-yl}pyridine-2,6-diamine

Starting from Preparation 2a following General procedure II and using 2-(2-methylphenoxy)propan-1-amine as the appropriate amine a mixture of Example 131 and Example 132 was obtained. The enantiomers were separated on OJ column using EtOH+0.05% DEA as eluent to obtain Example 131 as the first eluting enantiomer. HRMS (IT-TOF, ESI) m/z: Calculated for C₂H₂₄N₆O 388.2012, Found: 389.2103 [M+H]⁺ ee=99.8% (E1). Example 132 was obtained as the second eluting enantiomer. HRMS (IT-TOF, ESI) m/z: Calculated for C₂₂H₂₄N₆O 388.2012, Found: 389.2081 [M+H]⁺ ee=99.0% (E2).

### Example 133 4-{3-[(2S)-2-(2-fluorophenoxy)propyl]-2-methyl-3H-imidazo[4,5-b]pyridin-5-yl}pyridine-2,6-diamine

and

### Example 134 4-{3-[(2R)-2-(2-fluorophenoxy)propyl]-2-methyl-3H-imidazo[4,5-b]pyridin-5-yl}pyridine-2,6-diamine

Starting from Preparation 2a following General procedure II and using 2-(2-fluorophenoxy)propan-1-amine as the appropriate amine a mixture of Example 133 and Example 134 was obtained. The enantiomers were separated on CHIRALPAK AS-V column using 70:30 EtOH/heptane+0.05% DEA as eluent to obtain Example 133 as the first eluting enantiomer. HRMS (IT-TOF, ESI) m/z: Calculated for C₂₁H₂₁N₆OF 392.1761, Found: 393.1836 [M+H]V ee=99.8% (E1). Example 134 was obtained as the second eluting enantiomer. HRMS (IT-TOF, ESI) m/z: Calculated for C₂₁H₂₁NOF 392.1761, Found: 393.1852. [M+H]⁺ ee=99.6% (E2).

### Example 135 4-{2-methyl-3-[(2S)-2-(phenylsulfanyl)propyl]-3H-imidazo[4,5-b]pyridin-5-yl}pyridine-2,6-diamine

and

### Example 136 4-{2-methyl-3-[(2R)-2-(phenylsulfanyl)propyl]-3H-imidazo[4,5-b]pyridin-5-yl}pyridine-2,6-diamine

Starting from Preparation 2a following General procedure 11 and using 2-phenylsulfanylpropan-1-amine as the appropriate amine a mixture of Example 135 and Example 136 was obtained. The enantiomers were separated on CHIRALPAK AS-V column using 40:60 EtOH/heptane+0.05% DEA as eluent to obtain Example 135 as the first eluting enantiomer. HRMS (IT-TOF, ESI) m/z: Calculated for C₂₁H₂₂N₆S 390.1627, Found: 391.1701 [M+H]⁺ ee=99.8% (E1). Example 136 was obtained as the second eluting enantiomer. HRMS (IT-TOF, ESI) m/z: Calculated for C₂₁H₂₂N₆S 390.1627, Found: 391.1711 [M+H]⁺ ee=99.4% (E2).

### Example 137 4-{3-[(1S,2R)-2-benzylcyclopropyl]-2-methyl-3H-imidazo[4,5-b]pyridin-5-yl}pyridine-2,6-diamine

and

### Example 138 4-{3-[(1S,2S)-2-benzylcyclopropyl]-2-methyl-3H-imidazo[4,5-b]pyridin-5-yl}pyridine-2,6-diamine

and

### Example 139 4-{3-[(1R,2R)-2-benzylcyclopropyl]-2-methyl-3H-imidazo[4,5-b]pyridin-5-yl}pyridine-2,6-diamine

and

### Example 140 4-{3-[(R,2S)-2-benzylcyclopropyl]-2-methyl-3H-imidazo[4,5-b]pyridin-5-yl}pyridine-2,6-diamine

Starting from Preparation 2a following General procedure II and using 2-benzylcyclopropanamine as the appropriate amine a mixture of the cis-products (Example 137, Example 138) and the mixture of the trans-products (Example 139, Example 140) were obtained. The enantiomers of these mixtures were separated on CHIRALCEL OD column using 50:50 EtOH/heptane+0.05% DEA as eluent.

We obtained Example 137 as the first eluting enantiomer of the cis-mixture. HRMS (IT-TOF, ESI) m/z: Calculated for C₂₂H₂₂N₆ 370.1906, Found: 371.1966 [M+H]⁺ ee=99.8% (E1). Example 138 was obtained as the second eluting enantiomer of the cis-mixture. HRMS (IT-TOF, ESI) m/z: Calculated for C₂₂H₂₂N₆[M+H]⁺ 370.1906, Found: 371.1981 ee=99.8% (E2).

We obtained Example 139 as the first eluting enantiomer of the trans-mixture. HRMS (IT-TOF, ESI) m/z: Calculated for C₂₂H₂N₆ 370.1906, Found: 371.1983 [M+H]⁺ ee=99.6% (E1). Example 140 was obtained as the second eluting enantiomer of the trans-mixture. HRMS (IT-TOF, ESI) m/z: Calculated for C₂₂H₂₂N₆ 370.1906, Found: 371.1988 [M+H]⁺ ee=99.8% (E2).

### Example 141 4-{3-[(2S)-2-(2-methoxyphenoxy)propyl]-2-methyl-3H-imidazo[4,5-b]pyridin-5-yl}pyridine-2,6-diamine

and

### Example 142 4-{3-[(2R)-2-(2-methoxyphenoxy)propyl]-2-methyl-3H-imidazo[4,5-b]pyridin-5-yl}pyridine-2,6-diamine

Starting from Preparation 2a following General procedure II and using 2-(2-methoxyphenoxy)propan-1-amine as the appropriate amine a mixture of Example 141 and Example 142 was obtained. The enantiomers were separated on CHIRALPAK AS-H column using 50:50 1-PrOH/heptane+0.1% DEA as eluent to obtain Example 141 as the first eluting enantiomer. HRMS (TOF, ESI) m/z: Calculated for C₂₂H₂₄N₆O₂ 404.1961, Found: 405.2041 [M+H]⁺. Example 142 was obtained as the second eluting enantiomer. HRMS (TOF, ESI) m/z: Calculated for C₂₂H₂₄N₆O₂ 404.1961, Found: 405.2038 [M+H]⁺.

### Example 143 4-[2-methyl-3-(3-phenoxypropyl)-3H-imidazo[4,5-b]pyridin-5-yl]pyridine-2,6-diamine

Starting from Preparation 2a following General procedure II and using 3-phenoxypropan-1-amine as the appropriate amine Example 143 was obtained. HRMS (TOF, ESI) m/z: Calculated for C₂, H₂₂N₆O 374.1855, Found: 375.1946 [M+H]⁺.

### Example 144 4-(2,3-dibutyl-3H-imidazo[4,5-b]pyridin-5-yl)pyridine-2,6-diamine

Starting from Preparation 2b following General procedure II and using butan-1-amine as the appropriate amine Example 144 was obtained. HRMS (TOF, ESI) m/z: Calculated for C₁₉H₂₆N₆ 338.2219, Found: 339.2289 [M+H]⁺.

### Example 145 4-(2-butyl-3-cyclopentyl-3H-imidazo[4,5-b]pyridin-5-yl)pyridine-2,6-diamine

Starting from Preparation 2b following General procedure II and using cyclopentanamine as the appropriate amine Example 145 was obtained. HRMS (TOF, ESI) m/z: Calculated for C₂₀H₂₆N₆ 350.2219 Found: 351.2270 [M+H]⁺.

### Example 146 4-[2-butyl-3-(2-phenoxyethyl)-3H-imidazo[4,5-b]pyridin-5-yl]pyridine-2,6-diamine

Starting from Preparation 2b following General procedure 11 and using 2-phenoxyethanamine as the appropriate amine Example 146 was obtained. HRMS (TOF, ESI) m/z: Calculated for CH₂₆N₆O 402.2168 Found: 403.2235 [M+H]⁺.

### Example 147 4-(2-butyl-3-methyl-3H-imidazo[4,5-b]pyridin-5-yl)pyridine-2,6-diamine

Starting from Preparation 2b following General procedure II and using methanamine as the appropriate amine Example 0.147 was obtained. HRMS (TOF, ESI) m/z: Calculated for C₁₆H₂₀N₆ 296.1749, Found: 297.1824 [M+H]⁺.

### Example 148 4-(3-butyl-2-methyl-3H-imidazo[4,5-b]pyridin-5-yl)pyridine-2,6-diamine

Starting from Preparation 2a following General procedure II and using butan-1-amine as the appropriate amine Example 148 was obtained. HRMS (TOF, ESI) m/z: Calculated for Cl₆H₂₀N₆ 296.1749, Found: 297.1842 [M+H]⁺.

### Example 149 4-{3-[(1R)-1-(2-fluoropyridin-4-yl)ethyl]-2-methyl-3H-imidazo[4,5-b]pyridin-5-yl}pyridine-2,6-diamine

Starting from Preparation 2a following General procedure II and using (1R)-1-(2-fluoro-4-pyridyl)ethanamine as the appropriate amine Example 149 was obtained. HRMS (TOF, ESI) m/z: Calculated for C₁₉H₁₈N₇F 363.1608, Found: 364.1674 [M+H]⁺.

### Example 150 4-[3-(3-methoxypropyl)-2-methyl-3H-imidazo[4,5-b]pyridin-5-yl]pyridine-2,6-diamine

Starting from Preparation 2a following General procedure II and using 3-methoxypropan-1-amine as the appropriate amine Example 150 was obtained. HRMS (TOF, ESI) m/z: Calculated for C₁₆H₂₀N₆O 312.1699, Found: 313.1761 [M+H]⁺.

### Example 151 4-[3-(4-methoxybutyl)-2-methyl-3H-imidazo[4,5-b]pyridin-5-yl]pyridine-2,6-diamine

Starting from Preparation 2a following General procedure II and using 4-methoxybutan-1-amine as the appropriate amine Example 151 was obtained. HRMS (TOF, ESI) m/z: Calculated for C₁₇H₂₂N₆O 326.1855, Found: 327.1919 [M+H]⁺.

### Example 152 4-{3-[2-(3-methoxyphenyl)ethyl]-2-methyl-3H-imidazo[4,5-b]pyridin-5-yl}pyridine-2,6-diamine

Starting from Preparation 2a following General procedure II and using 2-(3-methoxyphenyl)ethanamine as the appropriate amine Example 152 was obtained. HRMS (TOF, ESI) m/z: Calculated for C₂₁H₂₂N₆O 374.1855, Found: 375.1919 [M+H]⁺.

### Example 153 4-[2-methyl-3-(2-phenoxyethyl)-3H-imidazo[4,5-b]pyridin-5-yl]pyridine-2,6-diamine

Starting from Preparation 2a following General procedure II and using 2-phenoxyethanamine as the appropriate amine Example 153 was obtained. HRMS (TOF, ESI) m/z: Calculated for C₂₀H₂₀N₆O 360.1699, Found: 361.1774. [M+H]⁺.

### Example 154 4-(3-ethyl-2-methyl-3H-imidazo[4,5-b]pyridin-5-yl)pyridine-2,6-diamine

Starting from Preparation 2a following General procedure II and using ethanamine as the appropriate amine Example 154 was obtained. HRMS (TOF, ESI) m/z: Calculated for C₁₄H₁₆N₆ 268.1436, Found: 269.1510 [M+H]⁺.

### Example 155 4-[3-(bicyclo[2.2.1]hept-2-yl)-2-methyl-3H-imidazo[4,5-b]pyridin-5-yl]pyridine-2,6-diamine

Starting from Preparation 2a following General procedure II and using norbornan-2-amine as the appropriate amine Example 155 was obtained. HRMS (TOF, ESI) m/z: Calculated for C₁₉H₂₂N₆ 334.1906, Found: 335.1981 [M+H]⁺.

### Example 156 4-(3-cyclopentyl-2-ethyl-3H-imidazo[4,5-b]pyridin-5-yl)pyridine-2,6-diamine

Starting from Preparation 2c following General procedure II and using cyclopentanamine as the appropriate amine Example 156 was obtained. HRMS (TOF, ESI) m/z: Calculated for C₁₈H₂₂N₆ 322.1906, Found: 323.1993 [M+H]⁺.

### Example 157 4-(3-butyl-2-ethyl-3H-imidazo[4,5-b]pyridin-5-yl)pyridine-2,6-diamine

Starting from Preparation 2c following General procedure II and using butan-1-amine as the appropriate amine Example 157 was obtained. HRMS (TOF, ESI) m/z: Calculated for C₁₇H₂₂N₆ 310.1906, Found: 311.1982 [M+H]⁺.

### Example 158 4-[3-(2,3-dihydro-1H-inden-2-yl)-2-ethyl-3H-imidazo[4,5-b]pyridin-5-yl]pyridine-2,6-diamine

Starting from Preparation 2c following General procedure II and using indan-2-amine as the appropriate amine Example 158 was obtained. HRMS (TOF, ESI) m/z: Calculated for C₂₂H₂₂N₆ 370.1906, Found: 371.1992 [M+H]⁺.

### Example 159 4-(2-ethyl-3-methyl-3H-imidazo[4,5-b]pyridin-5-yl)pyridine-2,6-diamine

Starting from Preparation 2c following General procedure 11 and using methanamine as the appropriate amine Example 159 was obtained. HRMS (TOF, ESI) m/z: Calculated for C₁₄H₁₆N₆ 268.1436, Found: 269.1512 [M+H]⁺.

### Example 160 4-(3-cyclopentyl-2-propyl-3H-imidazo[4,5-b]pyridin-5-yl)pyridine-2,6-diamine

Starting from Preparation 2d following General procedure II and using cyclopentanamine as the appropriate amine Example 160 was obtained. HRMS (TOF, ESI) m/z: Calculated for C₁₉H₂₄N₆ 336.2062 Found: 337.2150 [M+H]⁺.

### Example 161 4-(3-butyl-2-propyl-3H-imidazo[4,5-b]pyridin-5-yl)pyridine-2,6-diamine

Starting from Preparation 2d following General procedure II and using butan-1-amine as the appropriate amine Example 161 was obtained. HRMS (TOF, ESI) m/z: Calculated for C₁₈H₂₄N₆ 324.2062, Found: 325.2145 [M+H]⁺.

### Example 162 4-[3-(2-phenoxyethyl)-2-propyl-3H-imidazo[4,5-b]pyridin-5-yl]pyridine-2,6-diamine

Starting from Preparation 2d following General procedure 11 and using 2-phenoxyethanamine as the appropriate amine Example 162 was obtained. HRMS (IT-TOF, ESI) m/z: Calculated for C₂₂H₂₄N₆O 388.2012 Found: 389.2081 [M+H]⁺.

### Example 163 4-(3-methyl-2-propyl-31-imidazo[4,5-b]pyridin-5-yl)pyridine-2,6-diamine

Starting from Preparation 2d following General procedure 11 and using methanamine as the appropriate amine Example 163 was obtained. HRMS (TOF, ESI) m/z: Calculated for C₁₅H₁₈N₆ 282.1593, Found: 283.1663 [M+H]⁺.

### Example 164 4-(3-{2-[(5-chloropyridin-2-yl)oxy]ethyl}-2-methyl-3H-imidazo[4,5-b]pyridin-5-yl)pyridine-2,6-diamine

Starting from Preparation 3a following General procedure III and using 2-fluoro-5-chloropyridine as the appropriate aryl halide Example 164 was obtained. HRMS (IT-TOF, ESI) m/z: Calculated for C₁₉H₁₈N₇OCl 395.1261, Found: 396.1326 [M+H]⁺.

### Example 165 4-(3-{2-[(6-chloropyridin-2-yl)oxy]ethyl}-2-methyl-3H-imidazo[4,5-b]pyridin-5-yl)pyridine-2,6-diamine

Starting from Preparation 3a following General procedure II and using 2-fluoro-6-chloropyridine as the appropriate aryl halide Example 165 was obtained. HRMS (IT-TOF, ESI) m/z: Calculated for C₁₉H₁₈N₇OCl 395.1261, Found: 396.1331. [M+H]⁺.

### Example 166 4-(3-{2-[(3-chloropyridin-2-yl)oxy]ethyl}-2-methyl-3H-imidazo[4,5-b]pyridin-5-yl)pyridine-2,6-diamine

Starting from Preparation 3a following General procedure III and using 2-fluoro-3-chloropyridine as the appropriate aryl halide Example 166 was obtained. HRMS (IT-TOF, ESI) m/z: Calculated for C₁₉H₁₈N₇OCl 395.1261, Found: 396.1319 [M+H]⁺.

### Example 167 4-(3-{2-[(6-fluoropyridin-2-yl)oxy]ethyl}-2-methyl-3H-imidazo[4,5-b]pyridin-5-yl)pyridine-2,6-diamine

Starting from Preparation 3a following General procedure III and using 2,6-difluoropyridine as the appropriate aryl halide Example 167 was obtained. HRMS (TOF, ESI) m/z: Calculated for C₁₉H₁₈N₇OF 379.1557, Found: 380.1635 [M+H]⁺.

### Example 168 4-(3-{2-[(6-bromopyridin-2-yl)oxy]ethyl}-2-methyl-3H-imidazo[4,5-b]pyridin-5-yl)pyridine-2,6-diamine

Starting from Preparation 3a following General procedure 111 and using 2-fluoro-6-bromopyridine as the appropriate aryl halide Example 168 was obtained. HRMS (IT-TOF, ESI) m/z: Calculated for C₁₉H₁₈N₇OBr 439.0756, Found: 440.0823 [M+H]⁺.

### Example 169 4-(3-{2-[(3-bromopyridin-2-yl)oxy]ethyl}-2-methyl-3H-imidazo[4,5-b]pyridin-5-yl)pyridine-2,6-diamine

Starting from Preparation 3a following General procedure III and using 2-fluoro-3-bromopyridine as the appropriate aryl halide Example 169 was obtained. HRMS (IT-TOF, ESI) m/z: Calculated for C₁₉H₁₈N₇OBr 439.0756, Found: 440.0817 [M+H]⁺.

### Example 170 4-(3-{2-[(5-bromopyridin-2-yl)oxy]ethyl}-2-methyl-3H-imidazo[4,5-b]pyridin-5-yl)pyridine-2,6-diamine

Starting from Preparation 3a following General procedure III and using 2-fluoro-5-bromopyridine as the appropriate aryl halide Example 170 was obtained. HRMS (IT-TOF, ESI) m/z: Calculated for C₁₉H₁₈N₇OBr 439.0756, Found: 440.0811 [M+H]⁺.

### Example 171 4-(3-{2-[(5-fluoropyridin-2-yl)oxy]ethyl}-2-methyl-3H-imidazo[4,5-b]pyridin-5-yl)pyridine-2,6-diamine

Starting from Preparation 3a following General procedure III and using 2,5-difluoropyridine as the appropriate aryl halide Example 171 was obtained. HRMS (IT-TOF, ESI) m/z: Calculated for C₁₉H₁₈N₇OF 379.1557, Found: 380.1617 [M+H]⁺.

### Example 172 4-[3-(2-{[6-(fluoromethyl)pyridin-2-yl]oxy}ethyl)-2-methyl-3H-imidazo[4,5-b]pyridin-5-yl]pyridine-2,6-diamine

Starting from Preparation 3a following General procedure III and using 2-fluoro-6-fluoromethylpyridine as the appropriate aryl halide Example 172 was obtained. HRMS (TOF, ESI) m/z: Calculated for C₂₀H₂₀FN₇O 393.1713, Found: 394.1784 [M+H]⁺.

### Example 173 4-[3-(2-{[6-(difluoromethyl)pyridin-2-yl]oxy}ethyl)-2-methyl-3H-imidazo[4,5-b]pyridin-5-yl]pyridine-2,6-diamine

Starting from Preparation 3a following General procedure III and using 2-fluoro-6-difluoromethylpyridine as the appropriate aryl halide Example 173 was obtained. HRMS (TOF, ESI) m/z: Calculated for C₂₀H₁₉F₂N₇O 411.1619, Found: 412.1686 [M+H]⁺.

### Example 174 4-{2-methyl-3-[(2S)-2-(pyridin-2-yloxy)propyl]-3H-imidazo[4,5-b]pyridin-5-yl}pyridine-2,6-diamine

and

### Example 175 4-{2-methyl-3-[(2R)-2-(pyridin-2-yloxy)propyl]-3H-imidazo[4,5-b]pyridin-5-yl}pyridine-2,6-diamine

Starting from Preparation 3b following General procedure III and using 2-fluoropyridine as the appropriate aryl halide a mixture of Example 174 and Example 175 was obtained. The enantiomers were separated on CHIRALCEL OK column using 50:50 EtOH/heptane+0.05% DEA as eluent to obtain Example 174 as the first eluting enantiomer. HRMS (IT-TOF, ESI) m/z: Calculated for C₂₀H₂₁N₇O 375.1808, Found: 376.1867 [M+H]⁺ ee=99.8% (E1). Example 175 was obtained as the second eluting enantiomer. HRMS (IT-TOF, ESI) m/z: Calculated for C₂₀H₂₁N₇O 375.1808, Found: 376.1872 [M+H]⁺ ee=99.8% (E2).

### Example 176 4-(3-{(2S)-2-[(6-chloropyridin-2-yl)oxy]propyl}-2-methyl-3H-imidazo[4,5-b]pyridin-5-yl)pyridine-2,6-diamine

and

### Example 177 4-(3-{(2R)-2-[(6-chloropyridin-2-yl)oxy]propyl}-2-methyl-3H-imidazo[4,5-b]pyridin-5-yl)pyridine-2,6-diamine

Starting from Preparation 3b following General procedure III and using 2-fluoro-6-chloropyridine as the appropriate aryl halide a mixture of Example 176 and Example 177 was obtained. The enantiomers were separated on OJ column using EtOH+0.05% DEA as eluent to obtain Example 176 as the earlier eluting enantiomer. HRMS (IT-TOF, ESI) m/z: Calculated for C₂₀H₂₀N₇OCl 409.1418, Found: 410.1469 [M+H]⁺ ee=99.8% (E1). Example 177 was obtained as the later eluting enantiomer. HRMS (IT-TOF, ESI) m/z: Calculated for C₂₀H₂₀N₇OCl 409.1418, Found: 410.1482 [M+H]⁺ ee=98.8% (E2).

### Example 178 4-(3-{(2S)-2-[(5-fluoropyridin-2-yl)oxy]propyl}-2-methyl-3H-imidazo[4,5-b]pyridin-5-yl)pyridine-2,6-diamine

and

### Example 179 4-(3-{(2R)-2-[(5-fluoropyridin-2-yl)oxy]propyl}-2-methyl-3H-imidazo[4,5-b]pyridin-5-yl)pyridine-2,6-diamine

Starting from Preparation 3b following General procedure III and using 2,5-difluoropyridine as the appropriate aryl halide a mixture of Example 178 and Example 179 was obtained. The enantiomers were separated on AS column using 50:50 1-PrOH/heptane+0.1% DEA as eluent to obtain Example 178 as the earlier eluting enantiomer. HRMS (IT-TOF, ESI) m/z: Calculated for C₂₀H₂₀N₇O 393.1713, Found: 394.1780 [M+H]⁺ ee=99.4% (E1). Example 179 was obtained as the later eluting enantiomer. HRMS (IT-TOF, ESI) m/z: Calculated for C₂₀H₂₀N₇O 393.1713, Found: 394.1774 [M+H]⁺ ee=98.6% (E2).

### Example 180 4-(3-{(2S)-2-[(6-bromopyridin-2-yl)oxy]propyl}) 2-methyl-3H-imidazo[4,5-b]pyridin-5-yl)pyridine-2,6-diamine

and

### Example 181 4-(3-{(2R)-2-[(6-bromopyridin-2-yl)oxy]propyl}-2-methyl-3H-imidazo[4,5-b]pyridin-5-yl)pyridine-2,6-diamine

Starting from Preparation 3b following General procedure III and using 2-fluoro-6-bromopyridine as the appropriate aryl halide a mixture of Example 180 and Example 181 34) was obtained. The enantiomers were separated on CHIRALPAK AS-H column using 40:60 EtOH/heptane+0.1% DEA as eluent to obtain Example 180 as the earlier eluting enantiomer. HRMS (IT-TOF, ESI) m/z: Calculated for C₂₀H₂₀N₇OBr 453.0913, Found: 454.0970 [M+H]⁺ ee=99.8% (E1). Example 181 was obtained as the later eluting enantiomer. HRMS (IT-TOF, ESI) m/z: Calculated for C₂₀H₂₀N₇OBr 453.0913, Found: 454.0967 [M+H]⁺ ee=99.0% (E2).

### Example 182 4-(3-{(2S)-2-[(3-bromopyridin-2-yl)oxy]propyl}-2-methyl-3H-imidazo[4,5-b]pyridin-5-yl)pyridine-2,6-diamine

and

### Example 183 4-(3-{(2R)-2-[(3-bromopyridin-2-yl)oxy]propyl}-2-methyl-3H-imidazo[4,5-b]pyridin-5-yl)pyridine-2,6-diamine

Starting from Preparation 3b following General procedure III and using 2-fluoro-3-bromopyridine as the appropriate aryl halide a mixture of Example 182 and Example 183 was obtained. The enantiomers were separated on CHIRALPAK AS-H column using 70:30 2-PrOH/heptane+0.1% DEA as eluent to obtain Example 182 as the earlier eluting enantiomer. HRMS (IT-TOF, ESI) m/z: Calculated for C₂₀H₂₀N₇OBr 453.0913, Found: 454.0963 [M+H]⁺ ee=99.8% (E1). Example 183 was obtained as the later eluting enantiomer. HRMS (IT-TOF, ESI) m/z: Calculated for C₂₀H₂₀N₇OBr 453.0913, Found: 454.0968 [M+H]⁺ ee=99.0% (E2).

### Example 184 4-(3-{(2S)-2-[(5-chloropyridin-2-yl)oxy]propyl}-2-methyl-3H-imidazo[4,5-b]pyridin-5-yl)pyridine-2,6-diamine

and

### Example 185 4-(3-{(2R)-2-[(5-chloropyridin-2-yl)oxy]propyl}-2-methyl-3H-imidazo[4,5-b]pyridin-5-yl)pyridine-2,6-diamine

Starting from Preparation 3b following General procedure III and using 2-fluoro-5-chloropyridine as the appropriate aryl halide a mixture of Example 184 and Example 185 was obtained. The enantiomers were separated on OJ column using 50:50 EtOH/heptane+0.05% DEA as eluent to obtain Example 184 as the earlier eluting enantiomer. HRMS (IT-TOF, ESI) m/z: Calculated for C₂₀H₂₀N₇OCl 409.1418, Found: 410.1469. [M+H]⁺ ee=99.8% (E1). Example 185 was obtained as the later eluting enantiomer. HRMS (TOF, ESI) m/z: Calculated for C₂₀H₂₀ClN₇O 409.1418, Found: 410.1471 [M+H]⁺ ee 99.8% (E2).

### Example 186 4-(3-{(2S)-2-[(5-bromopyridin-2-yl)oxy]propyl}-2-methyl-3H-imidazo[4,5-b]pyridin-5-yl)pyridine-2,6-diamine

and

### Example 187 4-(3-((2R)-2-[(5-bromopyridin-2-yl)oxy]propyl-2-methyl-3H-imidazo[4,5-b]pyridin-5-yl)pyridine-2,6-diamine

Starting from Preparation 3b following General procedure III and using 2-fluoro-5-bromopyridine as the appropriate aryl halide a mixture of Example 186 and Example 187 was obtained. The enantiomers were separated on OJ column using 60:40 EtOH/heptane+0.05% DEA as eluent to obtain Example 186 as the earlier eluting enantiomer. HRMS (IT-TOF, ESI) m/z: Calculated for C₂₀H₂₀N_{T}OBr 453.0913 Found: 454.0967 [M+H]⁺. ee=99.8% (E1). Example 187 was obtained as the later eluting enantiomer. HRMS (IT-TOF, ESI) m/z: Calculated for C₂₀H₂₀N₇OBr 453.0913 Found: 454.0983 [M+H]⁺ ee=99.2% (E2).

### Example 188 4-(3-{(2S)-2-[(6-fluoropyridin-2-yl)oxy]propyl}-2-methyl-3H-imidazo[4,5-b]pyridin-5-yl)pyridine-2,6-diamine

and

### Example 189 4-(3-{(2R)-2-[(6-fluoropyridin-2-yl)oxy]propyl}-2-methyl-3H-imidazo[4,5-b]pyridin-5-yl)pyridine-2,6-diamine

Starting from Preparation 3b following General procedure 1.11 and using 2,6-difluoropyridine as the appropriate aryl halide a mixture of Example 188 and Example 189 was obtained. The enantiomers were separated on CHIRALCEL OJ-H column using EtOH+0.1% DEA as eluent to obtain Example 188 as the earlier eluting enantiomer. HRMS (IT-TOF, ESI) m/z: Calculated for C₂₀H₂₀N₇OF 393.1713, Found: 394.1779 [M+H]⁺ ee=99.8% (E1). Example 189 was obtained as the later eluting enantiomer. HRMS (IT-TOF, ESI) m/z: Calculated for C₂₀H₂₀N₇OF 393.1713, Found: 394.1773 [M+H]⁺ ee=−99.6% (E2).

### Example 190 4-(3-{(2S)-2-[(3-chloropyridin-2-yl)oxy]propyl}-2-methyl-3H-imidazo[4,5-b]pyridin-5-yl)pyridine-2,6-diamine

and

### Example 191 4-(3-{(2R)-2-[(3-chloropyridin-2-yl)oxy]propyl}-2-methyl-3H-imidazo[4,5-b]pyridin-5-yl)pyridine-2,6-diamine

Starting from Preparation 3b following General procedure III and using 2-fluoro-3-chloropyridine as the appropriate aryl halide a mixture of Example 190 and Example 191 was obtained. The enantiomers were separated on CHIRALPAK AS-V column using 70:30 2-PrOH/heptane+0.05% DEA as eluent to obtain Example 190 as the earlier eluting enantiomer. HRMS (IT-TOF, ESI) m/z: Calculated for C₂₀H₂₀N₇OCl 409.1418, Found: 410.1477 [M+H]⁺ ee=99.8% (E1). Example 191 was obtained as the later eluting enantiomer. HRMS (IT-TOF, ESI) m/z: Calculated for C₂₀H₂₀N₇OCl 409.1418, Found: 410.1492 [M+H]⁺ ee=99.8% (E2).

### Example 192 4-(3-{(2S)-2-[(3,6-difluoropyridin-2-yl)oxy]propyl}-2-methyl-3H-imidazo[4,5-b]pyridin-5-yl)pyridine-2,6-diamine

and

### Example 193 4-(3-{(2R)-2-[(3,6-difluoropyridin-2-yl)oxy]propyl}-2-methyl-3H-imidazo[4,5-b]pyridin-5-yl)pyridine-2,6-diamine

Starting from Preparation 3b following General procedure III and using 2,3,6-trifluoropyridine as the appropriate aryl halide a mixture of Example 192 and Example 193 was obtained. The enantiomers were separated on CHIRALCEL OK column using 60:40 EtOH/heptane+0.05% DEA as eluent to obtain Example 192 as the earlier eluting enantiomer. HRMS (TOF, ESI) m/z: Calculated for C₂₀H₁₉F₂N₇O 411.1699, Found: 412.1694 [M+H]⁺ ee=99.8% (E1). Example 193 was obtained as the later eluting enantiomer. HRMS (TOF, ESI) m/z: Calculated for C₂₀H₁₉F₂N₇O 411.1619, Found: 412.1700 [M+H]⁺ ee=99.8% (E2).

### Example 194 4-[2-methyl-3-(2-{[6-(trifluoromethyl)pyridin-2-yl]oxy}ethyl)-3H-imidazo[4,5-b]pyridin-5-yl]pyridine-2,6-diamine

Starting from Preparation 3a following General procedure IV and using 6-trifluoromethyl-2-pyridone as the appropriate phenol analog Example 194 was obtained. HRMS (IT-TOF, ESI) m/z: Calculated for C₂₀H₁₈N₇OF₃ 429.1525, Found: 430.1608 [M+H]⁺.

### Example 195 4-(2-methyl-3-{2-[(6-methylpyridin-2-yl)oxy]ethyl}-3H-imidazo[4,5-b]pyridin-5-yl)pyridine-2,6-diamine

Starting from Preparation 3a following General procedure IV and using 6-methyl-2-pyridone as the appropriate phenol analog Example 195 was obtained. HRMS (TOF, ESI) m/z: Calculated for C₂₀H₂₁N₇O 375.1808, Found: 376.1872 [M+H]⁺.

### Example 196 4-(3-{2-[(6-aminopyridin-2-yl)oxy]ethyl}-2-methyl-3H-imidazo[4,5-b]pyridin-5-yl)pyridine-2,6-diamine

Starting from Preparation 3a following General procedure IV and using 6-amino-2-pyridone as the appropriate phenol analog Example 196 was obtained. HRMS (IT-TOF, ESI) m/z: Calculated for C₁₉H₂₀N₈O 376.1760, Found: 377.1826 [M+H]⁺.

### Example 197 6-{2-[5-(2,6-diaminopyridin-4-yl)-2-methyl-3H-imidazo[4,5-b]pyridin-3-yl]ethoxy}pyridine-2-carbonitrile

Starting from Preparation 3a following General procedure IV and using 6-cyano-2-pyridone as the appropriate phenol analog Example 197 was obtained. HRMS (IT-TOF, ESI) m/z: Calculated for C₂₀H₁₈N₈O 386.1604, Found: 387.1655 [M+H]⁺.

### Example 198 4-{3-[2-(4-fluorophenoxy)ethyl]-2-methyl-3H-imidazo[4,5-b]pyridin-5-yl}pyridine-2,6-diamine

Starting from Preparation 3a following General procedure IV and using 4-fluorophenol as the appropriate phenol analog Example 198 was obtained. HRMS (TOF, ESI) m/z: Calculated for C₂₀H₁N₆OF 378.1604, Found: 379.1683 [M+H]⁺.

### Example 199 4-{3-[2-(3-fluorophenoxy)ethyl]-2-methyl-3H-imidazo[4,5-b]pyridin-5-yl}pyridine-2,6-diamine

Starting from Preparation 3a following General procedure IV and using 3-fluorophenol as the appropriate phenol analog Example 0.199 was obtained. HRMS (TOF, ESI) m/z: Calculated for C₂₀H₁₉N₆OF 378.1604, Found: 379.1621 [M+H]⁺.

### Example 200 4-{3-[2-(3-chlorophenoxy)ethyl]-2-methyl-3H-imidazo[4,5-b]pyridin-5-yl)}pyridine-2,6-diamine

Starting from Preparation 3a following General procedure IV and using 3-chlorophenol as the appropriate phenol analog Example 200 was obtained. HRMS (TOF, ESI) m/z: Calculated for C₂₀H₁₉N₆OCl 394.1309, Found: 395.1308 [M+H]⁺.

### Example 201 4-{2-methyl-3-[2-(3-methylphenoxy)ethyl]-3H-imidazo[4,5-b]pyridin-5-yl}pyridine-2,6-diamine

Starting from Preparation 3a following General procedure IV and using 3-methylphenol as the appropriate phenol analog Example 201 was obtained. HRMS (TOF, ESI) m/z: Calculated for C₂₁H₂₂N₆O 374.1855, Found: 375.1908 [M+H]⁺.

### Example 202 4-{3-[2-(3-methoxyphenoxy)ethyl]-2-methyl-3H-imidazo[4,5-b]pyridin-5-yl}pyridine-2,6-diamine

Starting from Preparation 3a following General procedure IV and using 3-methoxyphenol as the appropriate phenol analog Example 202 was obtained. HRMS (TOF, ESI) m/z: Calculated for C₂₁H₂₂N₆O₂ 390.1804, Found: 391.1810 [M+H]⁺.

### Example 203 4-{3-[2-(2-fluorophenoxy)ethyl]-2-methyl-3H-imidazo[4,5-b]pyridin-5-yl}pyridine-2,6-diamine

Starting from Preparation 3a following General procedure IV and using 2-fluorophenol as the appropriate phenol analog Example 203 was obtained. HRMS (TOF, ESI) m/z: Calculated for C₂₀H₁₉N₆OF 378.1604, Found: 379.1685 [M+H]⁺.

### Example 204 4-{3-[2-(2-chlorophenoxy)ethyl]-2-methyl-3H-imidazo[4,5-b]pyridin-5-yl}pyridine-2,6-diamine

Starting from Preparation 3a following General procedure IV and using 2-chlorophenol as the appropriate phenol analog Example 204 was obtained. HRMS (TOF, ESI) m/z: Calculated for C₂₀H₁₉N₆OCl 394.1309, Found: 395.1387 [M+H]⁺.

### Example 205 4-{2-methyl-3-[2-(2-methylphenoxy)ethyl]-3H-imidazo[4,5-b]pyridin-5-yl}pyridine-2,6-diamine

Starting from Preparation 3a following General procedure IV and using 2-methylphenol as the appropriate phenol analog Example 205 was obtained. HRMS (TOF, ESI) m/z: Calculated for C₂₁H₂₂N₆O 374.1855, Found: 375.1929 [M+H]⁺.

### Example 206 4-{3-[2-(2-methoxyphenoxy)ethyl]-2-methyl-3H-imidazo[4,5-b]pyridin-5-yl}pyridine-2,6-diamine

Starting from Preparation 3a following General procedure IV and using 2-methoxyphenol as the appropriate phenol analog Example 206 was obtained. HRMS (TOF, ESI) m/z: Calculated for C₂₁H₂₂N₆O₂ 390.1804, Found: 391.1888 [M+H]⁺.

### Example 207 4-{3-[2-(4-chlorophenoxy)ethyl]-2-methyl-3H-imidazo[4,5-b]pyridin-5-yl}pyridine-2,6-diamine

Starting from Preparation 3a following General procedure IV and using 4-chlorophenol as the appropriate phenol analog Example 207 was obtained. HRMS (TOF, ESI) m/z: Calculated for C₂₀H₁₉N₆OCl 394.1309, Found: 395.1389 [M+H]⁺.

### Example 208 4-{2-methyl-3-[2-(4-methylphenoxy)ethyl]-3H-imidazo[4,5-b]pyridin-5-yl}pyridine-2,6-diamine

Starting from Preparation 3a following General procedure IV and using 4-methylphenol as the appropriate phenol analog Example 208 was obtained. HRMS (TOF, ESI) m/z: Calculated for C₂₁H₂₂N₄O 374.1855, Found: 375.1937 [M+H]⁺.

### Example 209 4-{3-[2-(4-methoxyphenoxy)ethyl]-2-methyl-3H-imidazo[4,5-b]pyridin-5-yl}pyridine-2,6-diamine

Starting from Preparation 3a following General procedure IV and using 4-methoxyphenol as the appropriate phenol analog Example 209 was obtained. HRMS (TOF, ESI) m/z: Calculated for C₂₁H₂N₆O₂ 390.1804, Found: 391.1883 [M+H]⁺.

### Example 210 4-{3-[2-(1H-indol-5-yloxy)ethyl]-2-methyl-3H-imidazo[4,5-b]pyridin-5-yl}pyridine-2,6-diamine

Starting from Preparation 3a following General procedure IV and using 1H-indol-5-ol as the appropriate phenol analog Example 210 was obtained. HRMS (TOF, ESI) m/z: Calculated for C₂₂H₂₁N₇O 399.1808, Found: 400.1848 [M+H]⁺.

### Example 211 4-{3-[2-(2-methoxy-5-methylphenoxy)ethyl]-2-methyl-3H-imidazo[4,5-b]pyridin-5-yl}pyridine-2,6-diamine

Starting from Preparation 3a following General procedure IV and using 2-methoxy-5-methylphenol as the appropriate phenol analog Example 211 was obtained. HRMS (TOF, ESI) m/z: Calculated for C₂₂H₂₄N₆O₂ 404.1961, Found: 405.2022 [M+H]⁺.

### Example 212 4-{3-[2-(2,6-difluorophenoxy)ethyl]-2-methyl-3H-imidazo[4,5-b]pyridin-5-yl}pyridine-2,6-diamine

Starting from Preparation 3a following General procedure IV and using 2,6-difluorophenol as the appropriate phenol analog Example 212 was obtained. HRMS (TOF, ESI) m/z: Calculated for C₂₀H₁₈N₆OF₂ 396.1510, Found: 397.1570 [M+H]⁺.

### Example 213 4-{3-[2-(2,6-dimethoxyphenoxy)ethyl]-3H-imidazo[4,5-b]pyridin-5-yl}pyridine-2,6-diamine

Starting from Preparation 3a following General procedure IV and using 2,6-dimethoxyphenol as the appropriate phenol analog Example 213 was obtained. HRMS (TOF, ESI) m/z: Calculated for C₂₂H₂₄N₆O₃ 420.1910, Found: 421.1979 [M+H]⁺.

### Example 214 4-(2-methyl-3-{2-[2-(propan-2-yloxy)phenoxy]ethyl}-3H-imidazo[4,5-b]pyridin-5-yl)pyridine-2,6-diamine

Starting from Preparation 3a following General procedure IV and using 2-isopropoxyphenol as the appropriate phenol analog Example 214 was obtained. HRMS (TOF, ESI) m/z: Calculated for C₂₃H₂₆N₆O₂ 418.2117, Found: 419.2195 [M+H]⁺.

### Example 215 4-{3-[2-(2-ethoxyphenoxy)ethyl]-2-methyl-3H-imidazo[4,5-b]pyridin-5-yl}pyridine-2,6-diamine

Starting from Preparation 3a following General procedure IV and using 2-ethoxyphenol as the appropriate phenol analog Example 215 was obtained. HRMS (TOF, ESI) m/z: Calculated for C₂₂H₂₄N₆O₂ 404.1961, Found: 405.2030 [M+H]⁺.

### Example 216 4-{2-methyl-3-[2-(pyridin-3-yloxy)ethyl]-3H-imidazo[4,5-b]pyridin-5-yl}pyridine-2,6-diamine

Starting from Preparation 3a following General procedure IV and using 3-hydroxypyridine as the appropriate phenol analog Example 216 was obtained. HRMS (TOF, ESI) m/z: Calculated for C₁₉H₁₉N₇O 361.1651, Found: 362.1724 [M+H]⁺.

### Example 217 4-{2-methyl-3-[2-(pyridin-2-yloxy)ethyl]-3H-imidazo[4,5-b]pyridin-5-yl}pyridine-2,6-diamine

Starting from Preparation 3a following General procedure IV and using 2-pyridone as the appropriate phenol analog Example 217 was obtained. HRMS (TOF, ESI) m/z: Calculated for C₁₉H₁₉N₇O 361.1651, Found: 362.1731 [M+H]⁺.

### Example 218 4-(2-methyl-3-{2-[(1-methyl-1H-pyrazol-5-yl)oxy]ethyl}-3H-imidazo[4,5-b]pyridin-5-yl)pyridine-2,6-diamine

Starting from Preparation 3a following General procedure IV and using 1-methyl-1H-pyrazol-5-ol as the appropriate phenol analog Example 218 was obtained. HRMS (TOF, ESI) m/z: Calculated for C₁₈H₂₀N₈O 364.1760, Found: 365.1822 [M+H]⁺.

### Example 219 4-{2-methyl-3-[2-(pyrimidin-2-yloxy)ethyl]-3H-imidazo[4,5-b]pyridin-5-yl}pyridine-2,6-diamine

Starting from Preparation 3a following General procedure IV and using pyrimidin-2(1H)-one as the appropriate phenol analog Example 219 was obtained. HRMS (TOF, ESI) m/z: Calculated for C₁₈H₁₈N₈O 362.1604, Found: 363.1675 [M+H]⁺.

### Example 220 4-(2-chloro-3-cyclopentyl-3H-imidazo[4,5-b]pyridin-5-yl)pyridine-2,6-diamine

Following General procedure V using cyclopentanamine and as the appropriate amine and omitting Step F Example 220 was obtained. HRMS (TOF, ESI) m/z: Calculated for C₁₆H₁₇N₆Cl 328.1203, Found: 329.1272 [M+H]⁺.

### Example 221 3-cyclopentyl-5-(2,6-diaminopyridin-4-yl)-3H-imidazo[4,5-b]pyridine-2-carbonitrile

Following General procedure V and using cyclopentanamine as the appropriate amine Example 221 was obtained. HRMS (TOF, ESI) m/z: Calculated for C₁₇H₁₇N₇ 319.1545, Found: 320.1623 [M+H]⁺.

### Example 222 5-(2,6-diaminopyridin-4-yl)-3-(2-phenoxyethyl)-3H-imidazo[4,5-b]pyridine-2-carbonitrile

Following General procedure V and using 2-phenoxyethanamine as the appropriate amine Example 222 was obtained. HRMS (TOF, ESI) m/z: Calculated for C₂₀H₁₇N₇O 371.1495, Found: 372.1575 [M+H]⁺.

### Example 223 5-(2,6-diaminopyridin-4-yl)-3-ethyl-3H-imidazo[4,5-b]pyridine-2-carbonitrile

Following General procedure V and using ethanamine as the appropriate amine Example 223 was obtained. HRMS (IT-TOF, ESI) m/z: Calculated for C₁₄H₁₃N₇ 279.1232, Found: 280.1315 [M+H]⁺.

### Example 224 3-cyclopropyl-5-(2,6-diaminopyridin-4-yl)-3H-imidazo[4,5-b]pyridine-2-carbonitrile

Following General procedure V and using cyclopropanamine as the appropriate amine Example 224 was obtained. HRMS (IT-TOF, ESI) m/z: Calculated for C₁₈H₃N₇ 291.1232, Found: 292.1303 [M+H]⁺.

### Example 225 5-(2,6-diaminopyridin-4-yl)-3-(prop-2-en-1-yl)-3H-imidazo[4,5-b]pyridine-2-carbonitrile

Following General procedure V and using prop-2-en-1-amine as the appropriate amine Example 225 was obtained. HRMS (IT-TOF, ESI) m/z: Calculated for C₁₈H₁₃N₇ 291.1232, Found: 292.1300 [M+H]⁺.

### Example 226 5-(2,6-diaminopyridin-4-yl)-3-(4,4,4-trifluorobutyl)-3H-imidazo[4,5-b]pyridine-2-carbonitrile

Following General procedure V and using 4,4,4-trifluorobutan-1-amine as the appropriate amine Example 226 was obtained. HRMS (IT-TOF, ESI) m/z: Calculated for C₁₁H₁₄N₇F₃ 361.1263, Found: 362.1338 [M+H]⁺.

### Example 227 5-(2,6-diaminopyridin-4-yl)-3-methyl-3H-imidazo[4,5-b]pyridine-2-carbonitrile

Following General procedure V and using methanamine as the appropriate amine Example 227 was obtained. HRMS (IT-TOF, ESI) m/z: Calculated for C₁₃H₁₁N₇ 265.1076, Found: 266.1139 [M+H]⁺.

### Example 228 4-[2-(difluoromethyl)-3-methyl-3H-imidazo[4,5-b]pyridin-5-yl]pyridine-2,6-diamine

Following General procedure VI starting from Preparation 4 and using ethyl difluoroacetate as the appropriate acetic acid derivative Example 228 was obtained. HRMS (TOF, ESI) m/z: Calculated for C₁₃H₂N₆F₂ 290.1092, Found: 291.1161 [M+H]⁺.

### Example 229 4-[3-methyl-2-(trifluoromethyl)-3H-imidazo[4,5-b]pyridin-5-yl]pyridine-2,6-diamine

Following General procedure VI starting from Preparation 4 and using trifluoroacetic acid as the appropriate acetic acid derivative Example 229 was obtained. HRMS (TOF, ESI) m/z: Calculated for C₁₃H₁₁N₆F₃ 308.0997, Found: 309.1078 [M+H]⁺.

### Example 230 N-[6-amino-4-(3-butyl-2-methyl-3H-imidazo[4,5-b]pyridin-5-yl)pyridin-2-yl]-2-phenoxyacetamide

To a solution of 200 mg of 4-(3-butyl-2-methyl-imidazo[4,5-b]pyridin-5-yl)-N-triphenylmethyl-pyridine-2,6-diamine (Preparation 5) (0.37 mmol, 1 eq.) and 155 μL triethyl amine (1.11 mmol, 3 eq.) in 4 mL dry THF, 41 μL 2-chloroacetyl chloride (0.51 mmol, 1.4 eq.) was added and the mixture was stirred until no further conversion was observed. The volatiles were removed under reduced pressure, the residue was dissolved in 5 mL dry DMF and then 70 my phenol (0.74 mmol, 2 eq.) and 154 mg potassium carbonate (1.11 mmol, 3 eq.) were added. The resulting mixture was stirred until no further conversion was observed. It was diluted with brine and extracted with DCM. The combined organic phases were dried over MgSO₄, filtered and the filtrate was concentrated under reduced pressure. The crude product was first purified by flash chromatography on silica column using DCM/MeOH (1.2% NH₃) as eluents during which the trityl group was also removed, followed by preparative reversed phase chromatography using 5 mM aqueous NH₄HCO₃ solution and MeCN as eluents to give Example 230. HRMS (TOF, ESI) m/z: Calculated for C₂₄H₂₆N₆O₂ 430.2117, Found: 431.2195 [M+H]⁺.

### Example 231 N-benzyl-4-(3-butyl-2-methyl-3H-imidazo[4,5-b]pyridin-5-yl)pyridine-2,6-diamine

To a solution of 269 mg of 4-(3-butyl-2-methyl-imidazo[4,5-b]pyridin-5-yl)-N²-triphenylmethyl-pyridine-2,6-diamine (Preparation 5) (0.5 mmol, 1 eq.) and 159 mg benzaldehyde (1.5 mmol, 3 eq.) in DMF/MeOH (3/2 mL) 113 mg sodium borohydride (3 mmol, 6 eq.) was added in small portions. The resulting mixture was stirred at 60° C. in the presence of 100 μL acetic acid until no further conversion was observed. The pH was set to 5 with 2 M aqueous HCl solution and the resulting mixture was stirred until no further conversion (detritylation) was observed. The mixture was neutralized with 10% aqueous K₂CO₃ solution, diluted with brine, and extracted with DCM. The combined organic phases were dried over MgSO₄, filtered and the filtrate was concentrated under reduced pressure. The crude product was purified via preparative reversed phase chromatography using 5 mM aqueous NH₄HCO₃ solution and MeCN as eluents to give Example 231. HRMS (TOF, ESI) m/z: Calculated for C₂₃H₂₆N₆ 386.2219, Found: 387.2295 [M+H]⁺.

### Example 232 N-[6-amino-4-(3-butyl-2-methyl-3H-imidazo[4,5-b]pyridin-5-yl)pyridin-2-yl]-2-cyclohexylacetamide

Starting from Example 148 following General procedure VII using cyclohexylacetyl chloride as the appropriate acid chloride Example 232 was obtained. HRMS (TOF, ESI) m/z: Calculated for C₂₄H₂N₆O 420.2638, Found: 421.2719 [M+H]⁺.

### Example 233 N-[6-amino-4-(3-butyl-2-methyl-3H-imidazo[4,5-b]pyridin-5-yl)pyridin-2-yl]-2-chlorobenzamide

Starting from Example 148 following General procedure VII using 2-chlorobenzoyl chloride as the appropriate acid chloride Example 233 was obtained. HRMS (TOF, ESI) m/z: Calculated for C₂₃H₂₃N₆OCl 434.1622, Found: 435.1702 [M+H]⁺.

### Example 234 N-[6-amino-4-(3-butyl-2-methyl-3H-imidazo[4,5-b]pyridin-5-yl)pyridin-2-yl]cyclohexanecarboxamide

Starting from Example 148 following General procedure VII using cyclohexanoyl chloride as the appropriate acid chloride Example 234 was obtained. HRMS (TOF, ESI) m/z: Calculated for C₂₃H₃₀N₆O 406.2481, Found: 407.2557 [M+H]⁺.

### Example 235 N-[6-amino-4-(3-butyl-2-methyl-3H-imidazo[4,5-b]pyridin-5-yl)pyridin-2-yl]-2-phenylacetamide

Starting from Example 148 following General procedure VII using phenylacetyl chloride as the appropriate acid chloride Example 235 was obtained. HRMS (TOF, ESI) m/z: Calculated for C₂₄H₂₆N₆O 414.2168, Found: 415.2246 [M+H]⁺.

### Example 236 4-(3-butyl-2-methyl-3H-imidazo[4,5-b]pyridin-5-yl)pyridin-2-amine

Starting from Preparation 6a following General procedure IX Example 236 was obtained. HRMS (IT-TOF, ESI) m/z: Calculated for C₁₆H₁₉N₅ 281.1640, Found: 282.1710 [M+H]⁺.

### Example 237 4-[3-(2-cyclohexylethyl)-2-methyl-3H-imidazo[4,5-b]pyridin-5-yl]pyridin-2-amine

Starting from Preparation 6b following General procedure IX Example 237 was obtained. HRMS (TOF, ESI) m/z: Calculated for C₂₀H₂₅N₅ 335.2110, Found: 336.2184. [M+H]⁺.

### Example 238 4-[3-(cyclopropylmethyl)-2-methyl-3H-imidazo[4,5-b]pyridin-5-yl]pyridin-2-amine

Starting from Preparation 6c following General procedure IX Example 238 was obtained. HRMS (TOF, ESI) m/z: Calculated for C₁₆H₁₇N₅ 279.1484, Found: 280.1562 [M+H]⁺.

### Example 239 4-[3-(but-3-en-1-yl)-2-methyl-3H-imidazo[4,5-b]pyridin-5-yl]pyridin-2-amine

Starting from Preparation 6d following General procedure IX Example 239 was obtained. HRMS (IT-TOF, ESI) m/z: Calculated for C₁₆H₁₇N₅ 279.1484, Found: 280.1552 [M+H]⁺.

### Example 240 4-[3-(3,3-difluorocyclobutyl)-2-methyl-3H-imidazo[4,5-b]pyridin-5-yl]-6-fluoropyridin-2-amine

Starting from Preparation 7a following General procedure X and using 6-amino-4-bromo-2-fluoropyridine as the appropriate aryl halide Example 240 was obtained. HRMS (TOF, ESI) m/z: Calculated for C₁₆H₁₄F₃N₅ 333.1201, Found: 334.1270 [M+H]⁺.

### Example 241 4-(3-cyclopropyl-2-methyl-3H-imidazo[4,5-b]pyridin-5-yl)-3,6-difluoropyridin-2-amine

Starting from Preparation 6e following General procedure X and using 6-amino-4-bromo-2,5-difluoropyridine as the appropriate aryl halide Example 241 was obtained. HRMS (TOF, ESI) m/z: Calculated for C₁₈H₁₃F₂N₅ 301.1139, Found: 302.1202 [M+H]⁺.

### Example 242 4-(3-cyclopropyl-2-methyl-3H-imidazo[4,5-b]pyridin-5-yl)-3,5-difluoropyridine-2,6-diamine

Starting from Preparation 6f following General procedure X and using 4-bromo-2,6-diamino-3,5-difluoropyridine as the appropriate aryl halide Example 242 was obtained. HRMS (TOF, ESI) m/z: Calculated for C₁₅H₄F₂N 316.1248, Found: 317.1310 [M+H]⁺.

### Pharmacological Study

### Example A: Kinase TR-FRET Assays

Inhibition of the enzymatic activity of human kinases was evaluated in a Time-Resolved Fluorescence Resonance Energy Transfer (TR-FRET) assay in 384-well reaction plates. In this assay, full-length human kinases from Carna Biosciences—DYRK1A (NM_001396, ref. 04-130; 2.0 ng/μl), DYRK1B (NM_004714, ref. 04-131; 1.2 ng/p), CLK1 (NM_001162407, ref. 04-126; 0.7 ng/μl), CDK9 (NM_001261, ref. 04-110; 0.9 ng/μl), or GSK3β (NM_001146156, ref. 04-141; 2.0 ng/μl—were incubated for 40 minutes (DYRK1A and DYRK1B) or 100 minutes (CLK1, CDK9 and GSK3β) at room temperature with ATP (Sigma A2383, 10 μM) and a ULight™-labelled human Myelin Basic Protein (MBP) peptide substrate (Perkin Elmer TRF0109, 100 nM) in a reaction buffer composed of 50 mM HEPES pH7.4, 1 mM EGTA, 10 mM MgCl₂, 2 mM DTT and 0.01% Tween20. Test compounds of the invention were added in reaction buffer at a range of concentrations from 0.1 nM to 30 μM. Following addition of EDTA (Sigma E7889, 10 mM) to stop the reaction, Europium-labelled mouse monoclonal antibody recognizing phospho-Thr232 in MBP (Perkin Elmer TRF0201, 1 nM) was added. After one hour, the reaction plates were read using a fluorescence reader (EnVision®, Perkin Elmer) at 620 nm and 665 nm (excitation at 340 nm); when the Europium donor fluorophore is excited by light at 340 nm, an energy transfer (620 nm) to the acceptor occurs, which will then emit light at 665 nm. The activity, and hence inhibition, of DYRK1A kinase activity is thus measured by the relative intensity of the emitted light. The IC₅₀ was calculated from the concentration-activity curve as the concentration of the test compound required for 50% inhibition of kinase activity. The results are presented in Table 1.

### Example B: Kinase ADP Assays

The activity of His-TEV-DYRK1A Kinase domain (aa127-485) was measured using the accumulation of ADP produced during the phosphorylation of the peptide substrate Woodtide (Zinnsser Analytic) using ATP (Sigma Aldrich A7699). The enzyme reaction was conducted in assay buffer (pH 7.4), containing 15 mM Hepes; 20 mM NaCl; 1 mM EGTA; 10 mM MgCl2; 0.02% Tween20 and 0.1 mg/ml Bovine-y-globulin. Test compounds of the invention were added in reaction buffer in a range of concentrations for 10 minutes at 30° C. in the presence of 20 nM DYRK1A enzyme, 40 μM peptide substrate and 20 μM ATP. Detection reagents (DiscoveRx 90-0083), ADP Hunter Plus Reagent A and then ADP Hunter Plus Reagent B were added. After a following 20 minutes incubation at 30° C., ADP Hunter Plus Stop Solution was added. The fluorescence intensity was measured at 590 nm. The IC₅₀ was calculated from the concentration-activity curve as the concentration of the test compound required for 50% inhibition of kinase activity. The results are presented in Table 1.

### Example C: Cellular DYRK1 Autophosphorylation Assay

On day 0, human U2-OS osteosarcoma cells were seeded in 12-well culture plates (100,000 cells per well) and incubated at 37° C. in the presence of 5% CO₂ in 1 ml McCoy's 5A (Modified) medium containing GlutaMAX™ (Gibco 36600), supplemented with 50 units/ml penicillin, 50 μg/ml streptomycin, 10 mM Hepes buffer, pH=7.4, and 10% foetal calf serum (FCS, Sigma F7524). On day 1, medium was replaced with 500 μl Optimem medium containing GlutaMAX™ (Gibco 51985), 150 ng of a pcDNA3.1 plasmid (Invitrogen) containing a sequence coding for full-length, wild-type human DYRK1A (NM_001396) with an HA tag, 0.3% lipofectamine (Invitrogen 18324-020), and 0.6% Plus reagent (Invitrogen Cat No 11514-015). After 5 hours, medium was replaced with 900 μl McCoy's 5A (Modified) medium containing GlutaMAX™ (Gibco 36600). On day 2, cells were exposed to a range of concentrations of the test compounds of the invention for 5 hours. Cells were then washed in phosphate-buffered saline solution and cell lysed in lysis buffer comprised of 150 mM NaCl, 20 mM Tris-HCl pH 7.4, 1% triton X-100, 1 mM EGTA, 1 mM EDTA and protease (1% v/v; 539134; Calbiochem) and phosphatase (1% v/v; 524625; Calbiochem) inhibitor cocktails (50 μl lysis buffer/well). The relative levels of phospho-Ser520-DYRK1A were assayed using either western blotting or the Mesoscale ELISA platform. For analysis by western blot, lysates were diluted into Laemmli sample buffer (Bio-Rad) containing 5% v/v β-mercaptoethanol, heated for 5 min at 95° C., and resolved on Tris-glycine gels or NuPage Bis-Tris gels (Novex; Invitrogen). Biotinylated molecular weight standards (Cell Signaling Technology) were included in all gels. Proteins were transferred to nitrocellulose membranes (Hybond, ECL; Amersham), which were blocked in Tris-buffered saline/0.1% tween 20 (TBST) containing 5% milk, and probed at 4° C. overnight with anti-phospho-Ser520-DYRK1A antibody (Eurogentec SE6974-75; 0.23 μg/ml in 5% BSA) or anti DYRK1A antibody (Abnova H00001859; 0.5 μg/ml in 5% milk). Peroxidase-conjugated secondary antibodies were diluted into 5% milk and applied to membranes for 1 h at 20° C. Chemiluminescence detection was performed using the ECL plus western blotting detection kit (Amersham) and was recorded on ECL plus hyperfilm (Amersham). Blots were scanned using the Bio-Rad GS-800 calibrated densitometer and quantitative analysis of western blots was performed using TotalLab software (Amersham). IC₅₀ values for inhibition of phospho-Ser520-DYRK1A were calculated from dose-response curves plotting the ratio between phospho-Ser520-DYRK1A and total DYRK1A signals at each concentration. For analysis by Mesoscale ELISA, lysates were transferred to BSA-blocked ELISA plates with pre-bound anti-HA capture antibodies (Novus biological NB600-364; 15 μg/ml) for 1 hour with shaking at RT. Anti-phospho-Ser520-DYRK1A antibody (Eurogentec SE6974-75; 2.3-3.0 mg/ml) and anti DYRK1A antibody (Abnova H00001859; 3 μg/ml) was then added for 1 hour at RT, followed by addition of Sulfa-TAG anti-rabbit detection antibody (ref MSD R32AB; 1 μg/ml) and Sulfa-TAG anti-mouse detection antibody (ref MSD R32-AC-1; 1 μg/ml). After a further 1 hour, Read Buffer was added and plates were read on the Sector Imager 2400 (Mesoscale). IC₅₀ values for inhibition of phospho-Ser520-DYRK1A were calculated from dose-response curves. The results showed that the compounds of the invention are powerful inhibitors of cellular DYRK1A Ser520 autophosphorylation. The results are presented in Table 1.

### Example D: Pharmacodynamic Assay in Tumor Xenografts for Inhibition of DYRK1A Autophosphorylation

For pharmacodynamics studies of inhibition of DYRK1A autophosphorylation, female SCID mice were injected subcutaneously with RS4; 11 human acute lymphoblastic leukemia cells. When tumors reached a size of 200-300 mm³, mice were randomized into homogeneous groups of 3 and given a single oral administration of the compounds of the invention at doses of up to 100 mg/kg. At various times after treatment, typically 2 hours and 6 hours, treated and control mice were sacrificed, tumors were excised and proteins were extracted in tissue lysis buffer comprised of 150 mM NaCl, 20 mM Tris-HCl pH 7.4, 1% triton X-100, 1 mM EGTA, 1 mM EDTA and protease (1% v/v; 539134; Calbiochem) and phosphatase (1% v/v; 524625; Calbiochem) inhibitor cocktails. The relative levels of phospho-Ser520-DYRK1A were assayed using western blotting. For this, lysates were diluted into Laemmli sample buffer (Bio-Rad) containing 5% v/v β-mercaptoethanol, heated for 5 min at 95° C., and resolved on Tris-glycine gels or NuPage Bis-Tris gels (Novex; Invitrogen). Biotinylated molecular weight standards (Cell Signaling Technology) were included in all gels. Proteins were transferred to nitrocellulose membranes (Hybond, ECL; Amersham), which were blocked in Tris-buffered saline/0.1% tween 20 (TBST) containing 5% milk, and probed at 4° C. overnight with anti-phospho-Ser520-DYRK1A antibody (Eurogentec SE6974-75; 0.23 μg/ml in 5% BSA) or anti DYRK1A antibody (Abnova H00001859; 0.5 μg/ml in 5% milk). Peroxidase-conjugated secondary antibodies were diluted into 5% milk and applied to membranes for 1 h at 20° C. Chemiluminescence detection was performed using the ECL plus western blotting detection kit (Amersham) and was recorded on ECL plus hyperfilm (Amersham). Blots were scanned using the Bio-Rad GS-800 calibrated densitometer and quantitative analysis of western blots was performed using TotalLab software (Amersham). The percentage inhibition of phospho-Ser520-DYRK1A as compared to the control tumors was calculated using the ratio between phospho-Ser520-DYRK1A and total DYRK1A signals at each dose. The results showed that the compounds of the invention are powerful inhibitors of tumor DYRK1A Ser520 autophosphorylation.

| | | phospho-Ser520- |
|---|---|---|
| | dose | DYRK1A |
| Compound | (mg/kg) | (% control at 2 h) |
| Example 9 | 3 | 44 |
| Example 154 | 3 | 28 |
| Example 42 | 3 | 35 |
| Example 53 | 3 | 49 |
| Example 71 | 3 | 33 |
| Example 101 | 3 | 25 |
| Example 103 | 3 | 41 |
| Example 106 | 9 | 46 |

### Example E: Efficacy Studies in Tumor Xenografts

For anti-tumor efficacy studies, female nude balb/c nu/nu mice were injected subcutaneously with A2780 human ovarian carcinoma cells. When tumors reached a size of approximately 150 mm³, mice were randomized into homogeneous groups of 8 and treated orally with the compounds of the invention at doses of at doses of up to 75 mg/kg once daily for 2 weeks. Anti-tumor efficacy was monitored by at least twice-weekly measurement of tumor sizes using calipers, and body weights were recorded in order to document potential general toxicity. Percentage tumor growth inhibition (TGI) on a given day was calculated using the formula: (1-[RTV(treated)/RTV(untreated)])×100, where RTV=relative tumor volume on the given day versus start of treatment. The results showed that the compounds of the invention are powerful inhibitors of tumor growth.

TABLE 1
IC₅₀ of Dyrk1/Clk1 inhibitor

| | IC₅₀ (μM) Dyrk1A | IC₅₀ (μM) Dyrk1A | IC₅₀ (μM) Dyrk1B | IC₅₀ (μM) Clk1 | IC₅₀ (μM) CDK9 | IC₅₀ (μM) P-Ser520- |
|---|---|---|---|---|---|---|
| | TR-FRET assay | ADP assay | TR-FRET assay | TR-FRET assay | TR-FRET assay | Dyrk1A -Cell assay |
| Example 1 | 0.013 | 0.010 | 0.004 | | 0.314 | 0.080 |
| Example 2 | 0.008 | 0.010 | 0.001 | | 0.383 | 0.200 |
| Example 3 | 0.020 | 0.020 | | | 2.573 | 0.230 |
| Example 4 | 0.015 | 0.011 | | | 1.257 | 0.250 |
| Example 5 | 0.006 | 0.020 | | | 6.555 | 1.000 |
| Example 6 | 0.155 | 0.493 | 0.149 | | 1.090 | |
| Example 7 | 0.070 | 0.079 | | | 2.380 | |
| Example 8 | 0.061 | 0.128 | | | | |
| Example 9 | 0.011 | 0.007 | 0.006 | 0.021 | 2.149 | 0.090 |
| Example 10 | 0.032 | 0.079 | 0.055 | | 4.575 | |
| Example 11 | 0.004 | 0.011 | | | 3.449 | 0.170 |
| Example 12 | 0.002 | 0.009 | 0.003 | | | 0.070 |
| Example 13 | 0.012 | 0.035 | 0.019 | | >10 | |
| Example 14 | 0.002 | 0.008 | 0.001 | 0.016 | 1.946 | 0.050 |
| Example 15 | 0.004 | 0.011 | 0.008 | | 0.470 | 0.110 |
| Example 16 | 0.153 | 0.367 | | | | |
| Example 17 | 0.006 | 0.011 | | | | 0.063 |
| Example 18 | 0.002 | 0.013 | | | | 0.108 |
| Example 19 | 0.003 | 0.012 | 0.003 | | 1.952 | 0.226 |
| Example 20 | 0.016 | 0.032 | 0.016 | | 2.921 | |
| Example 21 | 0.015 | 0.053 | | | 12.479 | |
| Example 22 | 0.069 | 0.364 | | | | |
| Example 23 | 0.002 | 0.006 | 0.008 | 0.012 | 0.570 | 0.017 |
| Example 24 | 0.005 | 0.008 | 0.015 | | 3.528 | |
| Example 25 | 0.007 | 0.010 | 0.006 | | 1.837 | 0.228 |
| Example 26 | 0.004 | 0.007 | 0.006 | | 1.823 | |
| Example 27 | 0.015 | 0.056 | 0.005 | | >10 | |
| Example 28 | 0.002 | 0.006 | 0.003 | 0.025 | 5.700 | 0.028 |
| Example 29 | 0.004 | 0.011 | 0.011 | | 3.435 | 0.057 |
| Example 30 | 0.017 | 0.018 | 0.125 | | 3.568 | |
| Example 31 | 0.009 | 0.010 | 0.002 | | 1.776 | 0.070 |
| Example 32 | 0.095 | 0.037 | | | | |
| Example 33 | 0.045 | 0.018 | | | | |
| Example 34 | 0.030 | 0.010 | 0.015 | | 1.396 | |
| Example 35 | 0.012 | 0.008 | | | | |
| Example 37 | 0.052 | 0.027 | | | | |
| Example 38 | 0.028 | 0.008 | | | | |
| Example 39 | 0.245 | 0.045 | | | | |
| Example 40 | 0.154 | 0.187 | | | | |
| Example 41 | 0.029 | 0.011 | 0.011 | | 2.708 | 0.131 |
| Example 42 | 0.012 | 0.008 | 0.010 | 0.053 | 2.938 | 0.045 |
| Example 43 | 0.116 | 0.109 | | | | |
| Example 44 | 0.019 | 0.011 | | | | 0.023 |
| Example 45 | 0.177 | 0.055 | | | | |
| Example 46 | 0.056 | 0.035 | | | | |
| Example 47 | 0.003 | 0.025 | 0.046 | | 0.728 | |
| Example 48 | 0.116 | 0.056 | | | | |
| Example 49 | 0.003 | 0.011 | 0.017 | 0.017 | 0.979 | 0.130 |
| Example 50 | 0.029 | 0.057 | | | 1.992 | |
| Example 51 | 0.006 | 0.007 | 0.001 | 0.029 | 4.372 | 0.038 |
| Example 52 | 0.006 | 0.006 | 0.008 | | | |
| Example 53 | 0.006 | 0.009 | 0.004 | 0.020 | 1.312 | 0.063 |
| Example 54 | 0.003 | 0.006 | 0.003 | | | 0.023 |
| Example 55 | 0.002 | 0.024 | 0.021 | | | 0.160 |
| Example 56 | 0.009 | 0.015 | | | 0.357 | |
| Example 57 | 0.026 | 0.085 | 0.005 | | >10 | |
| Example 58 | 0.003 | 0.013 | 0.013 | | >10 | 0.080 |
| Example 59 | 0.002 | 0.021 | 0.003 | | >10 | |
| Example 60 | 0.149 | 0.510 | | | | |
| Example 61 | 0.002 | 0.009 | 0.004 | | >10 | 0.155 |
| Example 62 | 0.166 | 0.781 | | | | |
| Example 63 | 0.003 | 0.008 | 0.007 | | >10 | 0.024 |
| Example 64 | 0.009 | 0.026 | 0.030 | | >10 | |
| Example 65 | 0.001 | 0.006 | 0.005 | 0.018 | >10 | 0.022 |
| Example 66 | 0.005 | 0.009 | 0.012 | | 0.365 | 0.072 |
| Example 67 | 0.104 | 0.903 | | | | |
| Example 68 | 0.029 | 0.193 | 0.132 | | >10 | |
| Example 69 | 0.002 | 0.007 | | | | |
| Example 70 | | 0.013 | | | | |
| Example 71 | 0.006 | 0.006 | 0.006 | 0.016 | 1.407 | 0.047 |
| Example 72 | 0.010 | 0.015 | 0.014 | 0.090 | >10 | 0.184 |
| Example 73 | 0.004 | 0.009 | 0.008 | 0.019 | 4.660 | 0.065 |
| Example 74 | 0.006 | 0.014 | 0.010 | 0.021 | 0.476 | 0.092 |
| Example 75 | 0.003 | 0.012 | 0.006 | 0.019 | 0.843 | 0.035 |
| Example 76 | | 0.040 | | | | |
| Example 77 | | 0.008 | | | | 0.077 |
| Example 78 | | 0.025 | | 0.018 | | 0.133 |
| Example 79 | | 0.021 | | 0.014 | | 0.068 |
| Example 80 | | 0.043 | | | | |
| Example 81 | | 0.021 | | 0.015 | | 0.094 |
| Example 82 | | 0.096 | | | | |
| Example 83 | | 0.007 | | | | 0.037 |
| Example 84 | | 0.019 | | | | 0.113 |
| Example 85 | | 0.016 | | | | 0.143 |
| Example 86 | | 0.019 | | | | 0.151 |
| Example 87 | | 0.017 | | | | |
| Example 88 | | 0.012 | | | | 0.103 |
| Example 89 | 0.004 | 0.014 | 0.009 | 0.024 | 1.464 | 0.068 |
| Example 90 | | 0.015 | | 0.016 | | 0.129 |
| Example 91 | 0.004 | 0.013 | 0.008 | 0.016 | 2.916 | 0.062 |
| Example 92 | 0.002 | 0.006 | 0.007 | 0.017 | 0.564 | 0.058 |
| Example 93 | | 0.007 | | | | 0.107 |
| Example 94 | 0.003 | 0.005 | 0.007 | 0.016 | 1.078 | 0.017 |
| Example 95 | 0.003 | 0.012 | 0.011 | 0.029 | 1.798 | 0.021 |
| Example 96 | 0.004 | 0.011 | 0.010 | 0.020 | 3.094 | 0.075 |
| Example 97 | | 0.015 | | 0.021 | | 0.097 |
| Example 98 | 0.004 | 0.007 | 0.009 | 0.015 | | 0.019 |
| Example 99 | | 0.016 | | | | 0.104 |
| Example 100 | | 0.006 | | 0.014 | | 0.006 |
| Example 101 | 0.003 | 0.006 | 0.007 | 0.016 | 1.256 | 0.006 |
| Example 102 | | 0.017 | | | | |
| Example 103 | 0.006 | 0.006 | 0.005 | 0.015 | 0.666 | 0.014 |
| Example 104 | | 0.045 | | | | |
| Example 105 | | 0.092 | | | | |
| Example 106 | 0.008 | 0.012 | 0.006 | 0.019 | >3 | 0.073 |
| Example 107 | 0.008 | 0.012 | 0.007 | 0.037 | >3 | 0.111 |
| Example 108 | | 0.017 | | 0.026 | | 0.109 |
| Example 109 | 0.010 | 0.030 | 0.015 | 0.025 | >3 | 0.220 |
| Example 110 | | 0.019 | | 0.020 | | 0.042 |
| Example 111 | | 0.012 | | | | |
| Example 112 | 0.006 | 0.005 | 0.014 | 0.025 | 2.620 | 0.090 |
| Example 113 | | 0.010 | | | | 0.243 |
| Example 114 | 0.005 | 0.005 | 0.009 | 0.019 | | 0.039 |
| Example 115 | 0.003 | 0.013 | 0.006 | 0.028 | 5.692 | 0.034 |
| Example 116 | | 0.056 | | | | 0.267 |
| Example 117 | | 0.006 | | | | 0.075 |
| Example 118 | | 0.031 | | | | |
| Example 119 | | 0.004 | | | | 0.105 |
| Example 120 | | 0.003 | | | | 0.161 |
| Example 121 | | 0.008 | | | | 0.041 |
| Example 122 | | 0.020 | | | | 0.122 |
| Example 123 | | 0.010 | | | | 0.082 |
| Example 124 | | 0.006 | | | | 0.061 |
| Example 125 | | 0.022 | | | | 0.151 |
| Example 126 | | 0.055 | | | | 0.271 |
| Example 127 | | 0.015 | | | | 0.089 |
| Example 128 | | 0.037 | | | | 0.163 |
| Example 129 | | 0.037 | | | | 0.130 |
| Example 130 | | 0.058 | | | | |
| Example 131 | | 0.016 | | | | 0.045 |
| Example 132 | | 0.038 | | | | 0.207 |
| Example 133 | | 0.015 | | | | 0.074 |
| Example 134 | | 0.038 | | | | 0.205 |
| Example 135 | | 0.016 | | | | 0.098 |
| Example 136 | | 0.028 | | | | 0.247 |
| Example 137 | | 0.048 | | | | 0.197 |
| Example 138 | | 0.052 | | | | 0.100 |
| Example 139 | | 0.005 | | | | 0.012 |
| Example 140 | | 0.007 | | | | 0.024 |
| Example 141 | | 0.010 | | | | 0.191 |
| Example 142 | | 0.037 | | | | |
| Example 143 | | 0.013 | | | | 0.037 |
| Example 144 | 0.008 | 0.016 | 0.012 | 0.238 | >3 | 0.128 |
| Example 145 | | 0.030 | | 0.115 | | 0.171 |
| Example 146 | | 0.089 | | | | |
| Example 147 | | 0.021 | | | | |
| Example 148 | 0.002 | 0.007 | 0.006 | 0.016 | 1.244 | 0.100 |
| Example 149 | 0.010 | 0.026 | 0.004 | | >10 | 0.330 |
| Example 150 | 0.006 | 0.019 | 0.014 | | 3.027 | 0.280 |
| Example 151 | 0.004 | 0.013 | 0.008 | 0.019 | 2.871 | 0.170 |
| Example 152 | 0.002 | 0.012 | 0.007 | | >10 | 0.220 |
| Example 153 | 0.001 | 0.010 | 0.003 | 0.016 | 1.019 | 0.035 |
| Example 154 | 0.004 | 0.007 | 0.002 | 0.023 | 3.386 | 0.020 |
| Example 155 | 0.012 | 0.005 | | | | |
| Example 156 | 0.013 | 0.017 | 0.016 | | 0.554 | 0.151 |
| Example 157 | 0.011 | 0.021 | 0.003 | 0.058 | >10 | 0.153 |
| Example 158 | 0.008 | 0.014 | 0.016 | | | |
| Example 159 | | 0.043 | | | | |
| Example 160 | 0.003 | 0.024 | 0.068 | | | 0.144 |
| Example 161 | | 0.020 | 0.005 | 0.218 | >10 | 0.143 |
| Example 162 | | 0.056 | | | | |
| Example 163 | | 0.033 | | | | |
| Example 164 | | 0.032 | | | | 0.152 |
| Example 165 | 0.003 | 0.009 | 0.005 | 0.015 | | 0.026 |
| Example 166 | | 0.085 | | | | 0.250 |
| Example 167 | | 0.011 | | | | 0.011 |
| Example 168 | 0.002 | 0.011 | 0.006 | 0.015 | | 0.032 |
| Example 169 | | 0.055 | | | | |
| Example 170 | | 0.035 | | | | 0.131 |
| Example 171 | | 0.018 | | | | 0.109 |
| Example 172 | | 0.007 | | | | 0.092 |
| Example 173 | | 0.007 | | | | 0.017 |
| Example 174 | 0.016 | 0.074 | 0.013 | 0.083 | >3 | |
| Example 175 | 0.007 | 0.020 | 0.016 | 0.040 | >3 | 0.131 |
| Example 176 | | 0.093 | | | | 0.223 |
| Example 177 | 0.005 | 0.014 | 0.005 | 0.017 | 0.554 | 0.042 |
| Example 178 | | 0.176 | | | | |
| Example 179 | | 0.023 | | | | 0.237 |
| Example 180 | 0.005 | 0.013 | 0.010 | 0.016 | 0.467 | 0.044 |
| Example 181 | | 0.081 | | | | |
| Example 182 | | 0.026 | | | | 0.175 |
| Example 183 | | 0.226 | | | | |
| Example 184 | | 0.165 | | | | |
| Example 185 | | 0.041 | | | | |
| Example 186 | | 0.154 | | | | |
| Example 187 | | 0.082 | | | | |
| Example 188 | | 0.065 | | | | 0.181 |
| Example 189 | 0.007 | 0.013 | 0.007 | 0.017 | 0.339 | 0.026 |
| Example 190 | | 0.032 | | | | 0.148 |
| Example 191 | | 0.200 | | | | |
| Example 192 | | 0.084 | | | | |
| Example 193 | | 0.028 | | | | |
| Example 194 | | 0.007 | | | | 0.020 |
| Example 195 | | 0.013 | | | | 0.106 |
| Example 196 | | 0.012 | | | | 0.141 |
| Example 197 | | 0.007 | | | | 0.054 |
| Example 198 | | 0.025 | | 0.017 | | 0.092 |
| Example 199 | 0.003 | 0.012 | 0.007 | 0.016 | 1.102 | 0.053 |
| Example 200 | 0.005 | 0.018 | 0.010 | 0.020 | 1.523 | 0.069 |
| Example 201 | | 0.021 | | | | |
| Example 202 | | 0.023 | | 0.018 | | 0.180 |
| Example 203 | | 0.017 | | 0.016 | | 0.148 |
| Example 204 | | 0.041 | | | | |
| Example 205 | | 0.030 | | | | |
| Example 206 | 0.005 | 0.017 | 0.007 | 0.028 | >3 | 0.094 |
| Example 207 | | 0.026 | | 0.021 | | 0.120 |
| Example 208 | | 0.016 | | | | 0.155 |
| Example 209 | | 0.025 | | | | |
| Example 210 | 0.003 | 0.008 | 0.006 | 0.018 | 1.005 | 0.047 |
| Example 211 | | 0.049 | | | | |
| Example 212 | 0.003 | 0.007 | 0.040 | 0.017 | >10 | 0.083 |
| Example 213 | | 0.069 | | | | |
| Example 214 | | 0.072 | | | | |
| Example 215 | | 0.066 | | | | |
| Example 216 | | 0.029 | | | | |
| Example 217 | 0.006 | 0.010 | 0.008 | | 0.881 | 0.089 |
| Example 218 | | 0.057 | | | | |
| Example 219 | | 0.040 | | | | |
| Example 220 | 0.003 | 0.011 | 0.013 | 0.019 | 0.724 | 0.075 |
| Example 221 | 0.027 | 0.008 | 0.012 | 0.016 | 0.317 | 0.043 |
| Example 222 | 0.018 | 0.023 | 0.031 | 0.062 | >3 | 0.244 |
| Example 223 | | 0.009 | | 0.020 | | 0.097 |
| Example 224 | | 0.009 | | 0.020 | | 0.057 |
| Example 225 | | 0.012 | | 0.021 | | |
| Example 226 | 0.005 | 0.013 | 0.010 | 0.023 | 0.713 | 0.148 |
| Example 227 | | 0.010 | | 0.042 | | 0.262 |
| Example 228 | 0.004 | 0.010 | 0.012 | | | |
| Example 229 | 0.061 | 0.081 | | | | |
| Example 230 | 0.221 | 0.928 | | | | |
| Example 231 | 0.011 | 0.046 | 0.003 | | >10 | |
| Example 233 | 0.224 | 0.303 | | | | |
| Example 234 | 0.090 | 0.236 | | | | |
| Example 235 | 0.013 | 0.207 | 0.091 | | >10 | |
| Example 236 | 0.013 | 0.023 | 0.018 | | | |
| Example 237 | 0.005 | 0.045 | 0.005 | | 0.718 | |
| Example 238 | 0.009 | 0.050 | 0.020 | | | |
| Example 239 | | 0.068 | | | | |
| Example 240 | | 0.016 | | | | 0.153 |
| Example 241 | | 0.098 | | | | |
| Example 242 | | 0.154 | | | | |

### Example F: Pharmaceutical Composition: Tablets

| | |
|---|---|
| 1000 tablets containing a dose of | 5 g |
| 5 mg of a compoundselected from | |
| Example 1 to 242 | |
| Wheat starch | 20 g |
| Maize starch | 20 g |
| Lactose | 30 g |
| Magnesium stearate | 2 g |
| Silica | 1 g |
| Hydroxypropylcellulose | 2 g |

1-33. (canceled)

34. A compound of formula (I): 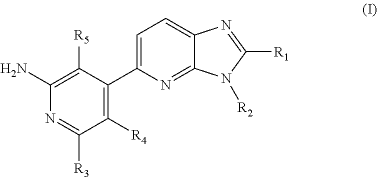
wherein:
R₁ represents a cyano group, a halogen atom, or a linear or branched (C₁-C₆)alkyl group optionally substituted by from one to three halogen atoms,
R₂ represents a hydrogen, a linear or branched (C₁-C₆)alkyl group, a linear or branched (C₂-C₆)alkenyl group, a linear or branched (C₂-C₆)alkynyl group, Cy₁, —(C₁-C₆)alkylene-[O]ₙ-Cy₁, —(C₁-C₆)alkenylene-[O]n-Cy₁, —(C₁-C₆)alkylene-NR-Cy₁, —(C₁-C₆)alkylene-S-Cy₁, —(C₀-C₆)alkylene-Cy₂-Cy₁, or —Cy₂-(C₁-C₆)alkylene-Cy₁, wherein the alkyl and alkylene moieties defined hereinbefore may be linear or branched,
R represents a hydrogen or a linear or branched (C₁-C₆)alkyl group,
n is 0 or 1,
R₃ represents a hydrogen atom, a halogen atom, —NR₆R_{6′}, —NH—(C₀-C₆)alkylene-Cy₃, —NH—CO—(C₀-C₆)alkylene-Cy₃, or —NH—CO—(C₀-C₆)alkylene-O-Cy₃,
R₄ and R₅, each independently represent a hydrogen or a halogen atom,
R₆ and R₆′, each independently represent a hydrogen or a linear or branched (C₁-C₆)alkyl group,
Cy₁, Cy₂ and Cy₃, independently of one another, represent a cycloalkyl group, a heterocycloalkyl group, an aryl or an heteroaryl group,
wherein:
“aryl” means a phenyl, naphthyl, biphenyl or indenyl group,
“heteroaryl” means any mono- or bi-cyclic group composed of from 5 to 10 ring members, having at least one aromatic moiety and containing from 1 to 4 hetero atoms selected from oxygen, sulphur and nitrogen,
“cycloalkyl” means any mono- or bi-cyclic, non-aromatic, carbocyclic group containing from 3 to 11 ring members, which may include fused, bridged or spiro ring systems,
“heterocycloalkyl” means any mono- or bi-cyclic, non-aromatic, condensed or spiro group composed of from 3 to 10 ring members and containing from 1 to 3 hetero atoms selected from oxygen, sulphur, SO, SO₂ and nitrogen, which may include fused, bridged or spiro ring systems,
“—(C₀-C₆)alkylene-” refers either to a covalent bond (—C₀alkylene-) or to an alkylene group containing 1, 2, 3, 4, 5 or 6 carbon atoms,
wherein the aryl, heteroaryl, cycloalkyl and heterocycloalkyl groups so defined and the alkyl, alkenyl, alkynyl, alkylene, alkenylene may be substituted by from 1 to 4 groups selected from linear or branched (C₁-C₆)alkyl, linear or branched (C₂-C₆)alkenyl, linear or branched (C₂-C₆)alkynyl, linear or branched (C₁-C₆)alkoxy, linear or branched (C₁-C₆)alkyl-S—, hydroxy, oxo (or N-oxide where appropriate), nitro, cyano, —C(O)—OR′, —C(O)—R′, —O—C(O)—R′, —C(O)—NR′R″, —NR′—C(O)—R″, —NR′R″, linear or branched (C₁-C₆)polyhaloalkyl, difluoromethoxy, trifluoromethoxy, or halogen, wherein R′ and R″ independently of one another represent a hydrogen atom or a substituted linear or branched (C₁-C₆)alkyl group,
its enantiomers and diastereoisomers, and addition salts thereof with a pharmaceutically acceptable acid or base.

35. The compound according to claim 34, wherein R₁ represents a methyl or a cyano group.

36. The compound according to claim 34, wherein R₄ and R₅ each represent a hydrogen atom.

37. The compound according to claim 34, wherein R₃ represents a NH₂ group.

38. The compound according to claim 34, wherein R₃ represents a hydrogen atom.

39. The compound according to claim 34, wherein R₂ represents a hydrogen, a linear or branched (C₁-C₆)alkyl group, a linear or branched (C₂-C₆)alkenyl group, a linear or branched (C₂-C₆)alkynyl group, —(C₁-C₆)alkylene-O-Cy₁, —(C₁-C₆)alkenylene-[O]ₙ-Cy₁, —(C₁-C₆)alkylene-NR-Cy₁, —(C₁-C₆)alkylene-S-Cy₁, —(C₀-C₆)alkylene-Cy₂-Cy₁, or —Cy₂-(C₂-C₆)alkylene-Cy₁, wherein the alkyl and alkylene moieties defined hereinbefore may be linear or branched.

40. The compound according to claim 34, wherein R₂ represents Cy₁, —(C₁-C₆)alkylene-Cy₁, —(C₀-C₆)alkylene-Cy₂-Cy₁, or —Cy₂-(C₁-C₆)alkylene-Cy₁.

41. The compound according to claim 40, wherein R₂ represents:
a cycloalkyl group,
or a —(C₁-C₆)alkylene-cycloalkyl or a —(C₁-C₆)alkylene-phenyl group,
or a -cycloalkylene-phenyl group or a -cycloalkylene-(C₁-C₆)alkylene-phenyl group,
wherein the cycloalkyl, cycloalkylene and phenyl groups so defined may be optionally substituted by from 1 to 4 groups selected from linear or branched (C₁-C₆)alkyl, linear or branched (C₂-C₆)alkenyl, linear or branched (C₂-C₆)alkynyl, linear or branched (C₁-C₆)alkoxy, linear or branched (C₁-C₆)alkyl-S—, hydroxy, oxo (or N-oxide where appropriate), nitro, cyano, —C(O)—OR′, —C(O)—R′, —O—C(O)—R′, —C(O)—NR′R″, —NR′—C(O)—R″, —NR′R″, linear or branched (C₁-C₆)polyhaloalkyl, difluoromethoxy, trifluoromethoxy, or halogen, wherein R′ and R″ independently of one another represent a hydrogen atom or a substituted linear or branched (C₁-C₆)alkyl group.

42. The compound according to claim 34, wherein R₂ represents a linear or branched (C₁-C₆)alkyl group, wherein the alkyl group so defined may be optionally substituted by from 1 to 4 groups selected from linear or branched (C₁-C₆)alkyl, linear or branched (C₂-C₆)alkenyl, linear or branched (C₂-C₆)alkynyl, linear or branched (C₁-C₆)alkoxy, linear or branched (C₁-C₆)alkyl-S—, hydroxy, oxo (or N-oxide where appropriate), nitro, cyano, —C(O)—OR′, —C(O)—R′, —O—C(O)—R′, —C(O)—NR′R″, —NR′—C(O)—R″, —NR′R″, linear or branched (C₁-C₆)polyhaloalkyl, difluoromethoxy, trifluoromethoxy, or halogen, wherein R′ and R″ independently of one another represent a hydrogen atom or a substituted linear or branched (C₁-C₆)alkyl group.

43. The compound according to claim 34, wherein R₂ represents a —(C₁-C₆)alkylene-O-Cy₁ group.

44. The compound according to claim 43, wherein R₂ represents a —(C₁-C₆)alkylene-O-pyridinyl group, wherein the pyridinyl group so defined may be optionally substituted by from 1 to 4 groups selected from linear or branched (C₁-C₆)alkyl, linear or branched (C₂-C₆)alkenyl, linear or branched (C₂-C₆)alkynyl, linear or branched (C₁-C₆)alkoxy, linear or branched (C₁-C₆)alkyl-S—, hydroxy, oxo (or N-oxide where appropriate), nitro, cyano, —C(O)—OR′, —C(O)—R′, —O—C(O)—R′, —C(O)—NR′R″, —NR′—C(O)—R″, —NR′R″, linear or branched (C₁-C₆)polyhaloalkyl, difluoromethoxy, trifluoromethoxy, or halogen, wherein R′ and R″ independently of one another represent a hydrogen atom or a substituted linear or branched (C₁-C₆)alkyl group.

45. The compound according to claim 34, which is selected from the group consisting of:
4-[2-methyl-3-(3-phenylcyclobutyl)-3H-imidazo[4,5-b]pyridin-5-yl]pyridine-2,6-diamine,
4-[3-(3,3-difluorocyclobutyl)-2-methyl-3H-imidazo[4,5-b]pyridin-5-yl]pyridine-2,6-diamine,
4-(3-{2-[(6-fluoropyridin-2-yl)oxy]ethyl}-2-methyl-3H-imidazo[4,5-b]pyridin-5-yl)pyridine-2,6-diamine,
4-{3-[(1R,2R)-2-benzylcyclopropyl]-2-methyl-3H-imidazo[4,5-b]pyridin-5-yl}pyridin-2,6-diamine,
4-[3-(3-fluorocyclobutyl)-2-methyl-3H-imidazo[4,5-b]pyridin-5-yl]pyridine-2,6-diamine,
4-(3-hexyl-2-methyl-3H-imidazo[4,5-b]pyridin-5-yl)pyridine-2,6-diamine,
4-(3-cyclobutyl-2-methyl-3H-imidazo[4,5-b]pyridin-5-yl)pyridine-2,6-diamine,
4-[3-(2-{[6-(difluoromethyl)pyridin-2-yl]oxy}ethyl)-2-methyl-3H-imidazo[4,5-b]pyridin-5-yl]pyridine-2,6-diamine,
4-[3-(5-methoxy-2,3-dihydro-1H-inden-2-yl)-2-methyl-3H-imidazo[4,5-b]pyridin-5-yl]pyridin-2,6-diamine,
4-(3-ethyl-2-methyl-3H-imidazo[4,5-b]pyridin-5-yl)pyridine-2,6-diamine,
4-[2-methyl-3-(2-{[6-(trifluoromethyl)pyridin-2-yl]oxy}ethyl)-3H-imidazo[4,5-b]pyridin-5-yl]pyridine-2,6-diamine,
4-{3-[2-(2-methoxycyclohexyl)ethyl]-2-methyl-3H-imidazo[4,5-b]pyridin-5-yl}pyridine-2,6-diamine,
4-(2-methyl-3-pentyl-3H-imidazo[4,5-b]pyridin-5-yl)pyridine-2,6-diamine,
4-(3-cyclohexyl-2-methyl-3H-imidazo[4,5-b]pyridin-5-yl)pyridine-2,6-diamine,
4-{2-methyl-3-[3-(methylsulfanyl)propyl]-3H-imidazo[4,5-b]pyridin-5-yl}pyridine-2,6-diamine,
4-{3-[(1R,2S)-2-benzylcyclopropyl]-2-methyl-3H-imidazo[4,5-b]pyridin-5-yl}pyridine-2,6-diamine,
4-{2-methyl-3-[2-(2-methylphenyl)ethyl]-3H-imidazo[4,5-b]pyridin-5-yl}pyridine-2,6-diamine,
4-(3-{2-[(6-chloropyridin-2-yl)oxy]ethyl}-2-methyl-3H-imidazo[4,5-b]pyridin-5-yl)pyridine-2,6-diamine,
4-(3-{(2R)-2-[(6-fluoropyridin-2-yl)oxy]propyl}-2-methyl-3H-imidazo[4,5-b]pyridin-5-yl)pyridine-2,6-diamine,
4-[2-methyl-3-(2,2,2-trifluoroethyl)-3H-imidazo[4,5-b]pyridin-5-yl]pyridine-2,6-diamine,
3-cyclopentyl-5-(2,6-diaminopyridin-4-yl)-3H-imidazo[4,5-b]pyridine-2-carbonitrile,
4-(3-cyclopropyl-2-methyl-3H-imidazo[4,5-b]pyridin-5-yl)pyridine-2,6-diamine, and
its enantiomers and diastereoisomers, and addition salts thereof with a pharmaceutically acceptable acid or base.

46. The compound according to claim 34, which is 4-(3-ethyl-2-methyl-3H-imidazo[4,5-b]pyridin-5-yl)pyridine-2,6-diamine.

47. The compound according to claim 34, which is 4-(2-methyl-3-pentyl-3H-imidazo[4,5-b]pyridin-5-yl)pyridine-2,6-diamine.

48. The compound according to claim 34, which is 4-{2-methyl-3-[2-(2-methylphenyl)ethyl]-3H-imidazo[4,5-b]pyridin-5-yl}pyridine-2,6-diamine.

49. The compound according to claim 34, which is 4-[2-methyl-3-(2,2,2-trifluoroethyl)-3H-imidazo[4,5-b]pyridin-5-yl]pyridine-2,6-diamine.

50. The compound according to claim 34, which is 4-(3-cyclopropyl-2-methyl-3H-imidazo[4,5-b]pyridin-5-yl)pyridine-2,6-diamine.

51. A pharmaceutical composition comprising the compound according to claim 34, or an addition salt thereof with a pharmaceutically acceptable acid or base, in combination with one or more pharmaceutically acceptable excipients.

52. A method of treating a condition selected from cancer, neurodegenerative disorders or metabolic disorders in a subject in need thereof, comprising administration of an effective amount of the compound according to claim 34, alone or in combination with one or more pharmaceutically acceptable excipients.

53. The method according to claim 52, wherein the cancer is selected from acute megakaryoblastic leukaemia (AMKL), acute lymphoblastic leukaemia (ALL), ovarian cancer, pancreatic cancer, gastrointestinal stromal tumours (GIST), osteosarcoma (OS), colorectal carcinoma (CRC), neuroblastoma and glioblastoma.

54. The method according to claim 52, wherein the neurodegenerative disorders are selected from Alzheimer's, Parkinson's and Huntington's diseases, Down's syndrome, mental retardation and motor defects.

55. A combination of the compound according to claim 34 with an anticancer agent selected from genotoxic agents, mitotic poisons, anti-metabolites, proteasome inhibitors, kinase inhibitors, signaling pathway inhibitors, phosphatase inhibitors, apoptosis inducers and antibodies.

56. A pharmaceutical composition comprising the combination according to claim 55 in combination with one or more pharmaceutically acceptable excipients.

57. A method of treating cancer in a subject in need thereof, comprising administration of the combination according to claim 55, alone or in combination with one or more pharmaceutically acceptable excipients.

58. A method of treating cancer necessitating radiotherapy in a subject in need thereof, comprising administration of the compound according to claim 34, alone or in combination with one or more pharmaceutically acceptable excipients.
